(19) United States
(12) Patent Application Publication
(10) Pub. No.: US 2023/0340044 A1
(43) Pub. Date: Oct. 26, 2023

(54) RECOMBINANT VARIANTS OF R-SPONDIN PROTEINS AND THEIR USE

(71) Applicants: DIOGENX, Marseille (FR); INSTITUT NATIONAL DE LA SANTÉ ET DE LA RECHERCHE MÉDICALE (INSERM), Paris (FR); UNIVERSITÉ CÔTE D'AZUR, Nice (FR); CENTRE NATIONAL DE LA RECHERCHE SCIENTIFIQUE (CNRS), Paris (FR)

(72) Inventors: Patrick COLLOMBAT, Tourrette Levens (FR); Paolo BOTTI, Marseille (FR)

(21) Appl. No.: 18/156,865

(22) Filed: Jan. 19, 2023

Related U.S. Application Data
(63) Continuation-in-part of application No. PCT/EP2022/069925, filed on Jul. 15, 2022.

(30) Foreign Application Priority Data
Jul. 15, 2021 (EP) 21305993.4
Mar. 18, 2022 (EP) 22305328.1

(51) Int. Cl. C07K 14/47; A61P 3/10; C12N 15/63

(52) U.S. Cl. CPC C07K 14/4705; A61P 3/10; C12N 15/63; C07K 2319/30

(57) ABSTRACT

The present invention relates to recombinant variants of R-spondin proteins and their use as a medicament, in particular for the treatment of diabetes.

### CROSS REFERENCE TO RELATED APPLICATIONS

This application is a continuation-in-part application of International Application No. PCT/EP2022/069925 filed Jul. 15, 2022, which claims priority to European Patent Application No. 21305993.4 filed Jul. 15, 2021, and European Patent Application No. 22305328.1 filed Mar. 18, 2022, the contents of each of which are hereby incorporated by references in their entireties.

### REFERENCE TO AN ELECTRONIC SEQUENCE LISTING

The contents of the electronic sequence listing (2023-01-19_DOG-01-PCTN_Sequence_ST.26.xml; Size: 153,170 bytes; and Date of Creation: Jan. 18, 2023) is herein incorporated by reference in its entirety.

The disclosure relates to recombinant variants of R-spondin proteins and their use as a medicament, in particular for the treatment of diabetes.

### BACKGROUND

Over the past few decades, diabetes has become one of the most widespread metabolic disorders with an epidemic dimension affecting almost 9% of the world's population (WHO, 2016). By the year 2049, the number of people affected by diabetes is projected to reach 600 million. Diabetes is characterized by high blood glucose levels, which, in most cases, result from the inability of the pancreas to secrete sufficient amounts of insulin. While type 1 diabetes (T1D) is caused by the autoimmune-mediated destruction of insulin-producing β-cells, type 2 diabetes (T2D) results from a resistance to insulin action and an eventual β-cell failure/loss over time.

Current treatments of diabetes fail to strictly restore normoglycemia and, in the case of T1D, even appear as rather palliative, replacing defective insulin secretion by exogenous insulin injections. Therefore, replenishing the pancreas with new functioning P-cells and/or maintaining the health of the remaining P-cells represent key strategies for the treatment of both conditions. However, to date, there is no available treatments preventing the loss of, or inducing the proliferation of pancreatic beta-cells, especially in human patients suffering from diabetes type 1.

Rspo1 belongs to a family of cysteine-rich secreted proteins, including also Rspo2, Rspo3 and Rspo4. They share a common structural architecture, including four structurally and functionally different domains as disclosed in FIG. 1. At the N-terminal, a signal peptide sequence ensures the correct entry of R-spondin proteins in the canonical secretory pathway. The mature secreted form contains two amino-terminal cysteine-rich furin-like repeats (FU1 and FU2), crucial for the interaction with R-spondin-specific receptors LGR (Leucine-rich repeat-containing G-protein coupled receptor) 4-6 (de Lau, W. B., Snel, B. & Clevers, H. C. Genome Biol 13, 242, doi:10.1186/gb-2012-13-3-242 (2012)) and Znrf3 (Zinc and ring finger 3). The central part of the protein contains one thrombospondin type-1 repeat domain (TSP1), involved in the interactions with specific components of the extracellular matrix, followed by a carboxy-terminal basic-amino acid rich domain, whose role has not yet been clarified. R-spondin proteins were reported to exert a key role in several biological processes, such as cell proliferation (Kim, K. A. et al. Science 309, 1256-1259, doi:10.1126/science.1112521 (2005). Da Silva, F. et al. Dev Biol 441, 42-51, doi:10.1016/j.ydbio.2018.05.024 (2018)), cell specification (Vidal, V. et al. Genes Dev 30, 1389-1394, doi:10.1101/gad.277756.116 (2016)) and sex determination (Chassot, A. A. et al. Hum Mol Genet 17, 1264-1277, doi:10.1093/hmg/ddn016 (2008)) and they have been reported as central regulators of the canonical WNT signaling pathway (also known as WNT/β-catenin or cWNT pathway) (Jin, Y. R. & Yoon, J. K. The R-spondin family of proteins: emerging regulators of WNT signaling. Int J Biochem Cell Biol 44, 2278-2287, doi:10.1016/j.biocel.2012.09.006 (2012)).

Despite the great deal of interest raised by the possible involvement of the cWNT pathway in pancreas maturation and function (Scheibner et al 2019, Curr Opin Cell Biol. 61:48-55), the roles and the contribution of R-spondin proteins have been poorly investigated in this organ.

In vitro analyses reported that, in the presence of Rspo1, β-cell proliferation and function are increased in the Min6 tumor-derived cell line (Wong, V. S., Yeung, A., Schultz, W. & Brubaker, P. L. R-spondin-1 is a novel beta-cell growth factor and insulin secretagogue. J Biol Chem 285, 21292-21302, doi:10.1074/jbc.M110.129874 (2010)). However, further more recent studies from the same group reported contradictory statements: Rspo1 deficiency in mice is associated with increased β-cell mass and enhanced glycemic controls (Wong, V. S., Oh, A. H., Chassot, A. A., Chaboissier, M. C. & Brubaker, P. L. Diabetologia 54, 1726-1734, doi:10.1007/s00125-011-2136-2 (2011) and Chahal et al 201, Pancreas Vol 43(1) pp 93-102).

In contrast to the latter studies, PCT/EP2021/050289 report that treatments with recombinant Rspo1 protein induce in vivo proliferation of functional pancreatic beta-cells, and improve glucose tolerance and increase glucose-stimulated insulin secretion (GSIS) in mice models of diabetes. In addition, it is suggested that upon near complete beta-cell ablation, the remaining beta-cells could be induced with Rspo1 protein administration to proliferate and reconstitute a functional beta-cells mass able to maintain euglycemia. Lastly, it is shown that Rspo1 can also induce human beta-cell proliferation opening new unexpected avenues for the treatment and prevention of diabetes in human with recombinant Rspo1 proteins.

RSPO1 critical residues for receptor binding and biological activities are described for example by Wang et Al. GENES & DEVELOPMENT 27:1339-1344, 2013; Xie et Al. EMBO reports VOL 14 | NO 12 | 2013; Xu et Al. THE JOURNAL OF BIOLOGICAL CHEMISTRY VOL. 290, NO. 4, pp. 2455-2465, Jan. 23, 2015. Key residues for binding and activity in both ZNRF3 and LGR4 receptors are indicated and also reported in FIG. 1.

There is still a need to further improve Rspo1-derived proteins for optimizing their developability properties and pharmacological properties for use as a biological medicament.

An object of the present disclosure is therefore to provide novel recombinant variants of R-spondin proteins for their use as a drug, e.g. in the treatment of diabetes.

### DETAILED DESCRIPTION

### Definitions

In order that the present disclosure may be more readily understood, certain terms are first defined. Additional definitions are set forth throughout the detailed description.

The term “amino acid” refers to naturally occurring and unnatural amino acids (also referred to herein as “non-naturally occurring amino acids”), e.g., amino acid analogues and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogues refer to compounds that have the same basic chemical structure as a naturally occurring amino acid, e.g., an alpha carbon that is bound to a hydrogen, a carboxyl group, an amino group, and an R group, e.g., homoserine, norieucine, methionine sulfoxide, methionine methyl sulfonium. Such analogues can have modified R groups (e.g., norieucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

Substitution refers to the replacement of a naturally occurring amino acid either with another naturally occurring amino acid or with an unnatural amino acid. For example, during chemical synthesis of a synthetic peptide, the native amino acid can be readily replaced by another naturally occurring amino acid or an unnatural amino acid. Alternatively, substitution may occur by mutating a genetic coding sequence in order to change a codon to another codon, encoding a different amino acid residue. The resulting polypeptide obtained by expression of the mutated coding sequence have one amino acid substitution (one amino acid replaced by another different amino acid).

As used herein, the term “protein” refers to any organic compounds made of amino acids arranged in one or more linear chains (also referred as “polypeptide chains”) and folded into a globular form. It includes proteinaceous materials or fusion proteins. The amino acids in such polypeptide chain may be joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation, PASylation and the like, incorporation of non-natural amino acids, amino acid modification for chemical conjugation or other molecules, etc. . . . .

The term “recombinant protein”, as used herein, includes proteins that are prepared, expressed, created or isolated by recombinant means, such as fusion proteins isolated from a host cell transformed to express the corresponding protein, e.g., from a transfectoma, etc. . . . .

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins. A protein fusion of the present disclosure thus includes at least one of R-spondin polypeptide or a variant thereof as described below, and at least one other moiety, the other moiety being a polypeptide other than a R-spondin polypeptide or a variant thereof as described below. In certain embodiments, the other moiety may also be a non-protein moiety, such as, for example, a polyethyleneglycol (PEG) moiety or other chemical moiety or conjugates. In preferred embodiments, the second moiety can be a Fc region of an antibody, and such fusion protein is therefore referred as a Fc fusion protein».

As used herein, the term “Fc region” is used to define the C-terminal region of an immunoglobulin heavy chain, including native sequence Fc region and variant Fc regions, preferably containing no more than 5, 10, 15, or 20 insertions, deletions, or substitutions of amino acid relative to the native human Fc region. The native human Fc region can be any of the IgG1, IgG2, IgG3, IgG4, IgA, IgA, IgD, IgE or IgM isotype. The human IgG heavy chain Fc region is generally defined as comprising the amino acid residue from position C226 or from P230 to the carboxyl-terminus of the IgG antibody. The numbering of residues in the Fc region being that of the EU index of Kabat. The C-terminal lysine (residue K447) of the Fc region may be removed, for example, during production or purification of an Fc fusion protein.

As used herein, the percent identity between the two sequences is a function of the number of identical positions shared by the sequences (i. e., % identity=number of identical positions/total number of positions ×100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm (NEEDLEMAN, and Wunsch).

The percent identity between two nucleotide or amino acid sequences may also be determined using for example algorithms such as EMBOSS Needle (pair wise alignment; available at www.ebi.ac.uk, Rice et al 2000 Trends Genet 16:276-277). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

As used herein, the term “subject” includes any human or nonhuman animal. The term “nonhuman animal” preferably includes mammals, such as nonhuman primates, sheep, dogs, cats, horses, etc.

As used herein, the term «R-spondin protein» refers to native R-spondin 1, R-spondin 2, R-spondin 3 or R-spondin 4 proteins (also referred herein as Rspo1, Rspo2, Rspo3, Rspo4 proteins) as encoded by corresponding Rspo1, Rspo2, Rspo3, Rspo4 gene respectively. Native R-spondin proteins typically include, from their N-terminal end to C-terminal end, a signal peptide (SP), two cystein-rich furin-like domains (FU1 and FU2), a N-glycosylation site between the FU2 domain and the TSP domain, a thrombospondin (TSP1) motif (TSP) and a basic amino acid rich (BR) domain, including potential O-glycosylation sites.

FIG. 1 provides an alignment of Rspo1, Rspo2, Rspo3 and Rspo4 amino acid sequence with a schematic view of the different domains FU1, FU2, TSP and BR. Throughout the present description, when referring to an amino acid position of Rspo1 or “the equivalent amino acid position” in Rspo2, Rspo3 and Rspo4, one should refer to the alignment of FIG. 1 to identify the “equivalent” amino acid position. Similarly, the Rspo2, Rspo3 or Rspo4 “equivalent” (or corresponding) domains of Rspo1 can be identified by referring to the alignment of FIG. 1. Full-length human Rspo1, Rspo2, Rspo3 and Rspo4 amino acid sequences (with their signal peptide) are described in SEQ ID Nos1-4 respectively. The amino acid sequences of FU1, FU2, TSP and BR domains of human Rspo1 are described in SEQ ID NO: 5-8 respectively. The amino acid sequences of FU1, FU2, TSP and BR domains of human Rspo2 are described in SEQ ID NO: 9-12 respectively. The amino acid sequences of FU1, FU2, TSP and BR domains of human Rspo3 are described in SEQ ID NO: 13-16 respectively. The amino acid sequences of FU1, FU2, TSP and BR domains of human Rspo4 are described in SEQ ID NO: 17-20 respectively.

As used herein, the term “chimeric protein” refers to a protein which includes the replacement of one or more domains within a particular protein by an equivalent domain of a protein of the same family. For example, a chimeric protein of Rspo1 may have the replacement of the Rspo1 FU1 domain by the corresponding FU1 domain of Rspo2, and comprises other domains (such as FU2, TSP, or BR domains) identical to the native Rspo1 protein.

As used herein, the term “hybrid domain” refers to a domain of a protein where some amino acid residues have been mutated to the equivalent residues of another member of the same family. For example, a hybrid FU1 domain of a R-spondin protein may correspond to Rspo2 FU1 domain with some mutations which have been made to replace one or more amino acid residues to the equivalent residues in Rspo2 FU1 domain.

The Variants of the Disclosure

The present disclosure relates to recombinant variants of native R-spondin protein comprising the following FU1, FU2, TSP and BR domains, wherein
FU1 is a domain having at least 80% identity to any of the FU1 domains of human Rspo1, Rspo2, Rspo3 or Rspo4 as shown in SEQ ID NO: 5, 9, 13 and 17 respectively,
FU2 is a domain having at least 80% identity to any of the FU2 domains of human Rspo1, Rspo2, Rspo3 or Rspo4 as shown in SEQ ID NO: 6, 10, 14, 18 respectively,
TSP is a domain having at least 80% identity to any of the TSP domains of human Rspo1, Rspo2, Rspo3 or Rspo4 as shown in SEQ ID NO: 7, 11, 15, 19, and,
BR is a domain having at least 80% identify to any of BR domains of human Rspo1, Rspo2, Rspo3, or Rspo4 as shown in SEQ ID NO: 8, 12, 16, 20.

Such recombinant variants of native R-spondin protein as defined above will be referred hereafter as “Variants of the Disclosure” or “Rspo1 Variants”.

For ease of reading, the sequences of the Variants of the Disclosure as described hereafter will always be described without any signal peptide sequence. However, all Variants disclosed herein may or may not include a N-terminal signal peptide (SP) sequence, and in particular one of the (SP) sequences of Rspo1, Rspo2, Rspo3 or Rspo4, typically the amino acid residue 1-20 of Rspo1 protein. A Variant of the Disclosure may also lack its normal signal sequence and has instead a different signal sequence replacing it. The choice of a signal sequence depends on the type of host cells in which the recombinant protein is to be produced, and a different signal sequence can replace the native signal sequence. Said signal peptide can be an optimized signal peptide, for example comprising or consisting of SEQ ID NO: 99 or 112. In addition, the Variants of the disclosure may comprise additional peptide sequence at their C-terminal, for example for purification. All Variants disclosed herein may also include a C-terminal tag, such as the polyhistidine tag of SEQ ID NO:90, including 6 histidine residues.

In specific embodiments of the Variants, each domain FU1, FU2, TSP and BR has at least 80% identity to the respective FU1, FU2, TSP and BR domains in human Rspo1 domain of SEQ ID NO:1.

In specific embodiments, the TSP and BR domains have 100% identity to corresponding human Rspo1 TSP and BR domains respectively, and the FU1 and FU2 domain amino acid sequences are at least 80% identical to human Rspo1 FU1 and FU2 corresponding amino acid sequences, the difference being due to amino acid substitutions.

In specific embodiments, the Variant of the Disclosure has no more than 1, 2, 3, 4, 5, 6, 7, 8, 9, or 10 amino acid substitutions in each of the FU1 or FU2 domain when aligned with corresponding human Rspo1 FU1 domain of SEQ ID NO: 5 and Rspo1 FU2 domain of SEQ ID NO:6 respectively.

In specific embodiments, the Variant of the Disclosure is a chimeric protein comprising
(i) a hybrid Rspo2 FU1 domain with no more than 1, 2, 3, 5, 6, 7, 8, 9, or 10 amino acid substitutions in the Rspo2 FU1 of SEQ ID NO: 9, and
(ii) the Rspo1 FU2 domain which is identical to SEQ ID NO:6 or includes no more than 1, 2, 3, 5, 6, 7, 8, 9, or 10 amino acid substitutions in the FU2 domain as compared to SEQ ID NO:6.

Preferably, the Variants of the Disclosure or their functional equivalents as described herein exhibit one or more of the following properties to a level at least similar to Rspo1 protein of SEQ ID NO: 41:
(i) They induce the proliferation of functional beta-cells to at least a similar level as reference human Rspo1 of SEQ ID NO:41, for example as determined in an in vitro Min6 beta-cell proliferation assay; and/or
(ii) They induce the proliferation of functional beta-cells to at least a similar level as reference human Rspo1 of SEQ ID NO:41, for example as determined in an in vivo beta-cell proliferation assay;
(iii) Optionally, they bind to LGR4 receptor with at least the same affinity as reference human Rspo1 of SEQ ID NO:41, as measured in Rspo1/LGR4 binding affinity in vitro assay, for example as determined by SPR or ELISA assay;
(iv) Optionally, they bind to ZNRF3 receptor with at least the same affinity as reference human Rspo1 of SEQ ID NO:41, as measured in Rspo1/ZNRF3 binding affinity in vitro assay, for example as determined by SPR or ELISA assay,
(v) Optionally, they potentiate Wnt/beta-catenin pathway as determined in vitro, for example as determined with the Top Flash assay.

The Variants of the Disclosure may advantageously be used as a medicament, in particular in treating diabetes in human and/or for inducing the proliferation of pancreatic beta-cells either in vivo or in vitro.

A. Amino Acid Deletions of the N-Terminal Residues

The inventors have surprisingly found that a deletion of the residues 21-31 of Rspo1 protein results in a more active protein in a Min6 proliferation assay as compared to the native Rspo1 protein of SEQ ID NO:41. Accordingly, in specific embodiments, the Variants of the Disclosure comprise a deletion of the first 10-14 N-terminal amino acid residues within the region 21-33 of Rspo1, or within an equivalent region in Rspo2, Rspo3, or Rspo4. Typically, a Variant of the Disclosure is a Rspo1 protein having a deletion of the 10-14 N-terminal amino acid residues within the region 21-33 of Rspo1.

More specifically, a Variant of the Disclosure comprises or consists of the protein of SEQ ID NO:24, which protein corresponds to human Rspo1 with a deletion of the amino acid residues 21-31 of Rspo1 (see the Variant #009 as disclosed in the Examples).

B. Amino Acid Substitution in the FU1 Domain to Increase Beta-Cell Proliferation.

The mutation R66A in Rspo1 FU1 domain has been described in Xie et Al. (EMBO reports VOL 14 | NO 12 | 2013) to decrease or abolish Rspo1 binding to ZNRF3.

As used herein, ZNRF3 refers to the E3 ubiquitin-protein ligase that acts as a negative regulator of the Wnt signaling pathway by mediating the ubiquitination and subsequent degradation of Wnt receptor complex components Frizzled and LRP6. It acts on both canonical and non-canonical Wnt signaling pathway. Rspondin proteins and in particular Rspo1 have been described to bind to ZNRF3 [and described as required for the signaling activity of R-spondin (Xie et Al. EMBO reports VOL 14 | NO 12 | 2013). ZNRF3 protein is described in Uniprot database at the accession number Q9ULT6 and NCBI Entrz Gene number 84133.

The inventors found that a mutation R66A in Rspo1, not only, did not abolish Rspo1 functionality in a Min6 proliferation assay, but even surprisingly improved its activity.

In specific embodiments, a Variant of the Disclosure advantageously comprises at least an amino acid substitution decreasing or abolishing ZNRF3 binding of residue R66 in human Rspo1 FU1 domain of SEQ ID NO:5, or of the equivalent arginine residue in human Rspo2, Rspo3, or Rspo4 FU1 domain sequence.

Binding affinity to ZNFR3 of any Variants may be compared with the corresponding binding affinity to ZNRF3 of reference human Rspo1 protein of SEQ ID NO:41. Methods to measure binding affinity to ZNRF3 are described in the Examples and include for example the ZNRF3 binding assay as disclosed in the Examples below. As used herein, a decrease in ZNRF3 binding means that the binding affinity is significantly lower, and preferably at least 20% lower, preferably at least 30% 40%, 50% than the corresponding binding affinity as measured with the reference Rspo1 of SEQ ID NO:41. The binding is abolished when binding is below detectable levels or similar to non-specific binding proteins.

Typically, said Variant of the Disclosure comprises Rspo1 FU1 domain of SEQ ID NO:5 having a single amino acid substitution of residue R66 decreasing or abolishing ZNRF3 binding, typically R66A. In specific embodiments, said Variant of the Disclosure comprises Rspo1 FU1 domain of SEQ ID NO:5 having a single amino acid substitution of residue R66 decreasing or abolishing ZNRF3 binding, typically R66A, and said FU2, TSP and BR domains are 95%, preferably 100% identical to Rspo1 FU2, TSP and BR domains respectively.

For example, the Variant of the Disclosure comprises or essentially consists of SEQ ID NO:23 (corresponding to Variant #008 as disclosed in the Examples below).

C. Amino Acid Substitutions in the FU1 and FU2 Domains to Increase Binding to LGR4 and/or ZNRF3

The Variants of the Disclosure may further include amino acid substitution in the FU1 and/or FU2 domain to increase its binding affinity to LGR4 and/or ZNRF3 as compared to native Rspo1 of SEQ ID NO:41.

Binding affinity to LGR4 and/or ZNFR3 of any Variants may be compared with the corresponding binding affinity to ZNRF3 or LGR4 of reference human Rspo1 protein of SEQ ID NO:41. Methods to measure binding affinity to ZNRF3 or LGR4 are described in the Examples, and include for example the ZNRF3 binding assay as disclosed in the Examples below. As used herein, an increase in ZNRF3 or LGR4 binding means that the binding affinity is significantly higher, and preferably at least 10% higher, preferably at least 20%, 30%, 40%, 50% than the corresponding binding affinity as measured with the reference Rspo1 of SEQ ID NO:41.

Amino acid residues relevant for binding affinity to LGR4 and/or ZNRF3 and possible amino acid mutations have been described in particular in Wang et Al. GENES & DEVELOPMENT 27:1339-1344, 2013; Xie et Al. EMBO reports VOL 14 | NO 12 | 2013; Xu et Al. THE JOURNAL OF BIOLOGICAL CHEMISTRY VOL. 290, NO. 4, pp. 2455-2465, Jan. 23, 2015.

In specific embodiments, said Variant of the Disclosure includes one or more amino acid substitutions at position H108, N109, E116, L118, P127, A128, S133, A136, G138, or S143 of Rspo1 or corresponding residues in Rspo2, Rspo3 or Rspo4 to increase binding affinity to LGR4. In more specific embodiments, said Variant of the Disclosure includes one or more of the following amino acid substitutions H108K, H108R, N109D, N109E, E116V; L118F; P127D; A128E; S133F; A136L; G138E; S143V of Rspo1 FU2 or corresponding residues in Rspo1, Rspo3 or Rspo4, preferably of Rspo1. More specifically, said Variant of the Disclosure comprises or essentially consists of SEQ ID NO:66 (corresponding to Variant #049 as described in the Examples), SEQ ID NO:70 (corresponding to Variant #054 as described in the Examples), SEQ ID NO:72 (corresponding to Variant #056 as described in the Examples), SEQ ID NO:67 (corresponding to Variant #050 as described in the Examples), SEQ ID NO:28 (corresponding to Variant #051 as described in the Examples), SEQ ID NO:74 (corresponding to Variant #068 as described in the Examples), or SEQ ID NO:75 (corresponding to Variant #069 as described in the Examples).

In specific embodiments, said Variant of the Disclosure includes one or more amino acid substitutions at position E45, L46, E49, V50, N51, K55, S57, 162, L63, D68, P77, F84, D85, N88, or 195 of Rspo1, or corresponding residues in Rspo2, Rspo3 or Rspo4 to increase binding affinity to ZNRF3. In specific embodiments, said Variant of the Disclosure includes one or more of the following amino acid substitutions L46S, E49K, V50D, K55R, S57Q, 162F, L63F, D68G, P77H, F84Y, D85Y, N88A, or 195A of Rspo1 FU1, or corresponding residues in Rspo1, Rspo3 or Rspo4, preferably of Rspo1. In more specific embodiments, said Variant of the Disclosure comprises or essentially consists of SEQ ID NO:65 (corresponding to Variant #048 as described in the Examples), SEQ ID NO:67 (corresponding to Variant 50 as described in the Examples), SEQ ID NO:68 (corresponding to Variant #052 as described in the Examples), SEQ ID NO:69 (corresponding to Variant #053 as described in the Examples), SEQ ID NO:71 (corresponding to Variant #055 as described in the Examples), SEQ ID NO:73 (corresponding to Variant #057 as described in the Examples).

In certain embodiments, the Variant of the disclosure comprises
(i) one or more amino acid substitutions at position E45, L46, E49, V50, N51, K55, S57, 162, L63, D68, P77, F84, D85, N88, or 195 of Rspo1, or corresponding residues in Rspo2, Rspo3 or Rspo4 to increase binding affinity to ZNRF3; for example one or more of the following amino acid substitutions L46S, E49K, V50D, K55R, S57Q, 162F, L63F, D68G, P77H, F84Y, D85Y, N88A, or 195A of Rspo1 FU1, or corresponding residues in Rspo1, Rspo3 or Rspo4, preferably of Rspo1, and,
(ii) one or more amino acid substitutions at position H108, N109, E116, L118, P127, A128, S133, A136, G138, or S143 of Rspo1 or corresponding residues in Rspo2, Rspo3 or Rspo4 to increase binding affinity to LGR4; for example, one or more of the following amino acid substitutions H108K, H108R, N109D, N109E, E116V; L118F; P127D; A128E; S133F; A136L; G138E; S143V of Rspo1 FU2 or corresponding residues in Rspo1, Rspo3 or Rspo4, preferably of Rspo1.

Examples of such Variants include Variant #050 (SEQ ID NO: 67), Variant #057 (SEQ ID NO: 73), and Variants #108, 4112.

D. Amino Acid Deletions or Substitutions of the N-Gylcosylation Site

The Variant of the Disclosure may also advantageously comprise an amino acid deletion or substitution to remove the N-glycosylation site in the FU2 domain so that the resulting Variant is not N-glycosylated, even when produced in a eukaryotic expression system such as a mammalian cell line, e.g. CHO or human cell line.

Preferably, said Variant of the Disclosure includes an amino acid substitution in residue N137 of Rspo1 or in an equivalent residue in Rspo2, Rspo3, or Rspo4 to suppress N-glycosylation at this position, for example said Variant includes the amino acid substitution N137Q or Rspo1 or equivalent amino acid substitution in Rspo2, Rspo3 or Rspo4. In specific embodiments, said Variant of the Disclosure comprises or essentially consists of SEQ ID NO:22 (corresponding to Variant #005 in the Examples below), SEQ ID NO:54 (corresponding to Variant #030 in the Examples below).

E. Chimeric or Hybrid R-Spondin Proteins

The Variants of the Disclosure also encompass chimeric R-spondin proteins, optionally further including one or more of the modifications (deletion or amino acid substitutions) as described in the previous sections B-D.

In specific embodiments, a chimeric Variant of the Disclosure comprises said FU1 domain which is 100% identical to Rspo2 FU1 domain of SEQ ID NO:9 and said FU2 domain which is selected among FU2 domains of Rspo1, Rspo3, Rspo4, or their functional variants with amino acid substitutions maintaining at least the same binding affinity to LGR4 (as compared to Rspo1 of SEQ ID NO:41 as reference), preferably said FU2 domain is 100% identical to Rspo1 FU2 domain of SEQ ID NO:6.

Accordingly, in specific embodiments, the Variant is a chimeric protein combining FU1 domain of Rspo2 with the other domains of Rspo1, Rspo3, Rspo4, or their functional equivalents.

In specific embodiments, the Variant is a chimeric protein which comprises at least the C-terminal region from amino acid residues 144-263 of Rspo1 (SEQ ID NO:49).

Typically, one example of such chimeric Variant is a variant comprising Rspo2 FU1 domain of SEQ ID NO:9 and Rspo1 FU2 domain of SEQ ID NO:6, and optionally Rspo1 TSP domain of SEQ ID NO:7 and Rspo1 BR domain of SEQ ID NO:8, preferably the variant of SEQ ID NO:25 (corresponding to Variant #034 as disclosed in the Examples) or the variant of SEQ ID NO:26 (corresponding to Variant #035 as disclosed in the Examples).

Another example of such chimeric variant is a variant comprising Rspo1 FU1 domain and Rspo3 FU2 domain, and optionally Rspo1 TSP domain of SEQ ID NO:7 and Rspo1 BR domain of SEQ ID NO:8, preferably the variant of SEQ ID NO:50 or 51 (corresponding to Variant #026 or Variant #027 as disclosed in the Examples).

Another example of such chimeric variant is a variant comprising Rspo3 FU1 domain and Rspo1 FU2 domain, and optionally Rspo1 TSP domain of SEQ ID NO:7 and Rspo1 BR domain of SEQ ID NO:8, preferably the variant of SEQ ID NO:52 or 53 (corresponding to Variant #028 or Variant #029 as disclosed in the Examples), or variant of SEQ ID NO:54 (corresponding to Variant #030, further including the N137Q mutation to suppress N-glycosylation).

Another example of such chimeric variant is a variant comprising Rspo2 FU1 and FU2 domains, and Rspo1 TSP domain of SEQ ID NO:7 and Rspo1 BR domain of SEQ ID NO:8, preferably the variant of SEQ ID NO:55 (corresponding to Variant #031 as disclosed in the Examples).

Another example of such chimeric variant is a variant comprising Rspo2 FU2 domain and the rest of the R-spondin protein from Rspo1, preferably the variant of SE ID NO:56 (corresponding to Variant #032 as disclosed in the Examples).

Another example of such chimeric variant is a variant comprising Rspo1 FU1 domain and Rspo2 FU2 domain, and optionally Rspo1 TSP domain of SEQ ID NO:7 and Rspo1 BR domain of SEQ ID NO:8, preferably the variant of SEQ ID NO:57 (corresponding to Variant #033 as disclosed in the Examples).

Another example of such chimeric variant is a variant comprising Rspo4 FU1 and FU2 domains, and Rspo1 TSP domain of SEQ ID NO:7 and Rspo1 BR domain of SEQ ID NO:8, preferably the variant of SEQ ID NO:58 (corresponding to Variant #036 as disclosed in the Examples).

Another example of such chimeric variant is a variant comprising Rspo4 FU2 domain and the rest of the R-spondin protein from Rspo1, preferably the variant of SE ID NO:59 (corresponding to Variant #037 as disclosed in the Examples).

Another example of such chimeric variant is a variant comprising Rspo1 FU1 domain and Rspo4 FU2 domain, and optionally Rspo1 TSP domain of SEQ ID NO:7 and Rspo1 BR domain of SEQ ID NO:8, preferably the variant of SEQ ID NO:60 (corresponding to Variant #038 as disclosed in the Examples).

Another example of such chimeric variant is a variant comprising Rspo4 FU1 domain and Rspo1 FU2 domain, and optionally Rspo1 TSP domain of SEQ ID NO:7 and Rspo1 BR domain of SEQ ID NO:8, preferably the variant of SEQ ID NO:61 or 62 (corresponding to Variant #039 or Variant #040 as disclosed in the Examples), Another example of such chimeric variant is a variant comprising Rspo2 FU1 domain and Rspo4 FU2 domain, and optionally Rspo1 TSP domain of SEQ ID NO:7 and Rspo1 BR domain of SEQ ID NO:8, preferably the variant of SEQ ID NO:63 (corresponding to Variant #042 as disclosed in the Examples).

In addition to the above chimeric proteins, it is also possible to optimize ZNRF3 binding activity of the chimeric protein by further replacing one or more amino acid residues in FU1 domain of Rspo2, with corresponding one or more amino acid residues of Rspo1 at positions known to be crucial for Rspo1/ZNRF3 binding affinity or to increase binding affinity to ZNRF3.

Typically, the Variant of the Disclosure comprises a hybrid FU1 domain having the same sequence as SEQ ID NO:5 except that it includes one or more amino acid substitutions at position E49, V50, D68, D85 to increase binding affinity to ZNRF3 as compared to the chimeric Variant of SEQ ID NO:25 or SEQ ID NO:26, preferably it is a hybrid Rspo1 FU1 domain of SEQ ID NO:5 except that it includes one or more amino acid substitutions at position E49, V50, D68, D85, preferably it includes one or more of the following amino acid substitutions E49K, V50D, D68G, D85G (corresponding to residues of Rspo1 FU1 domain), and further wherein the FU2 domain is selected among FU2 domains of Rspo1, Rspo2 Rspo3, Rspo4, or their functional variants with amino acid substitutions maintaining at least the same binding affinity to LGR4, preferably said FU2 domain is 100% identical to Rspo1 FU2 domain of SEQ ID NO:6.

In specific embodiment, the Variant of the Disclosure comprises a hybrid FU1 domain having the same sequence as SEQ ID NO:5 (i.e. Rspo1 FU1 domain) except that it includes one or more amino acid substitutions at position E49, V50, D68, and D85, preferably it includes the following amino acid substitutions 49K, 50D, 68G, 85G (corresponding to residues of Rspo2 FU1 domain), and further wherein the FU2 domain is 100% identical to Rspo1 FU2 domain of SEQ ID NO:6. For example, the Variant of the disclosure comprises or essentially consists of SEQ ID NO:27 (corresponding to Variant #047 as disclosed in the Examples).

In specific embodiments, the Variant of the Disclosure as disclosed above, further comprises hybrid Rspo1 FU1 and Rspo1 FU2 domains with one or more amino acid substitutions selected among E45L, E49K, V50D, K55R, D68G, D85G, N88A, H108K, and N109D. For example, the Variant of the disclosure comprises or essentially consists of SEQ ID NO:28 (corresponding to Variant #051 as disclosed in the Examples with E45L; E49K; V50D; K55R; D68G; D85G; N88A; H108K; and N109D mutations).

In specific embodiments, the Variant of the Disclosure comprises human Rspo2, Rspo3 or Rspo4 corresponding FU2 domain of SEQ ID NO. 10, 14 or 18, in combination with Rspo1 FU1 domain or hybrid Rspo1 FU1 domain for example including one or more amino acid substitutions at position E49, V50, D68 and D85 to increase binding to ZNRF3, such as E49K, V50D, D68G, D85G.

F. Amino Acid Substitutions in the BR Domain to Improve O-Glycosylation

The Variants of the Disclosure may further include one or more amino acid substitutions in the BR domain to modulate, optimize or improve O-glycosylation as compared to Rspo1 of SEQ ID NO:41.

Amino acid residues relevant for improving O-glycosylation may include, in particular, the amino acid residue T253, T258, S259 in Rspo1.

In specific embodiments, said Variant of the Disclosure includes one or more of the following amino acid substitutions at position G252, T253, L257, T258, S259, A260, A263 of Rspo1 or corresponding residues in Rspo2, Rspo3 or Rspo4 to improve O-glycosylation. More specifically, said Variant of the Disclosure includes a BR domain of Rspo1 with one or more of the following amino acid substitutions G252T, T253E, L257S, T258E, S259E, A260T, or A263T, S268E of Rspo1 or corresponding residues in Rspo2, Rspo3 or Rspo4.

In contrast, when the Variants is fused to a C-terminal polypeptide, then it may be advantageous to mutate the O-glycosylation site. According, in specific embodiments, the Variants of the disclosure are fusion protein and includes one or more of the following amino acid substitutions: T253A, T258A, S259A, and S268A.

In specific embodiments, a Variant of the Disclosure includes or essentially consists of the polypeptide of SEQ ID Nos76-86.

G. Truncated R-Spondin Active Proteins.

The inventors have surprisingly found that a deletion of the residues 21-31 and 245-263 of Rspo1 protein results a similar activity in a Min6 proliferation assay as compared to the native Rspo1 protein of SEQ ID NO:41. This protein represents the shortest active native Rspo1 protein. More specifically, a Variant of the Disclosure comprises or consists of the protein of SEQ ID NO:94, which protein corresponds to human Rspo1 fragment comprising amino acid residues from 32 to 244 of Rspo1 (see the Variant #116 as disclosed in the Examples).

H. Rspo3 for Use in Treating Diabetes

The inventors have further determined that the native protein Rspo3 activates pancreatic beta-cell proliferation in vivo to a level at least similar to Rspo1. Accordingly, in one aspect, the disclosure relates to a Rspo3 polypeptide (for example comprising SEQ ID NO:3) or a functional equivalent thereof, for use in treating diabetes (e.g. diabetes type I or II) and/or for inducing the proliferation of pancreatic beta-cells, either in vitro or in vivo.

Preferred Variants of the Disclosure

Table 1 describes preferred Variants of the Disclosure, their amino acid sequence and their main changes as compared to native R-spondin proteins.

TABLE 1

| | Amino acid | |
|---|---|---|
| Example | SEQ ID | Brief Description |
| #005 | SEQ ID NO: 22 | human Rspo1 w/o signal peptide with N137Q (non- |
| | | N-glycosylated) |
| #008 | SEQ ID NO: 23 | hRspo1 w/o signal peptide with R66A single mutation |
| #009 | SEQ ID NO: 24 | hRspo1 with deletion of residues 21-31 |
| #018 | | hRspo1 32-263 with N137Q (non-N-glycosylated) |
| #026 | SEQ ID NO: 50 | Chimeric: RSPO1Fu1_32-95-RSPO3Fu2_97-143- |
| | | RSPO1_144-263 |
| #027 | SEQ ID NO: 51 | Chimeric: RSPO1Fu1_32-90-RSPO3Fu2_92-143- |
| | | RSPO1_144-263 |
| #028 | SEQ ID NO: 52 | Chimeric: RSPO3Fu1_33-96-RSPO1Fu2_96-143- |
| | | RSPO1_144-263 |
| #029 | SEQ ID NO: 53 | Chimeric: RSPO3Fu1_33-91-RSPO1Fu2_91-143- |
| | | RSPO1_144-263 |
| #030 | SEQ ID NO: 54 | Chimeric. RSPO3Fu1_33-96-RSPO1Fu2_96-143- |
| | | RSPO1_144-263 N137Q mutation. No N-Glycosylation |
| #031 | SEQ ID NO: 55 | Chimeric: RSPO2Fu1-Fu2_32-142-RSPO1_144-263 |
| #032 | SEQ ID NO: 56 | Chimeric Fu2-Rspo2 into Rspo1 |
| #033 | SEQ ID NO: 57 | Chimeric: RSPO1Fu1_32-90-RSPO2Fu2_90-142- |
| | | RSPO1_144-263 |
| #034 | SEQ ID NO: 25 | Chimeric Rspo2 FU1 domain with Rspo1 FU2 domain |
| #035 | SEQ ID NO: 26 | Identical to #034 but with different FU1 and FU2 junction |
| #036 | SEQ ID NO: 58 | Chimeric: RSPO4Fu1-Fu2_27-136-RSPO1_144-263 |
| #037 | SEQ ID NO: 59 | Chimeric Fu2-Rspo4 into Rspo1 |
| #038 | SEQ ID NO: 60 | Chimeric: RSPO1Fu1_32-90-RSPO4Fu2_85-136- |
| | | RSPO1_144-263 |
| #039 | SEQ ID NO: 61 | Chimeric: RSPO4Fu1_27-89-RSPO1Fu2_96-143- |
| | | RSPO1_144-263 |
| #040 | SEQ ID NO: 62 | Chimeric: RSPO4Fu1_27-84-RSPO1Fu2_91-143- |
| | | RSPO1_144-263 |
| #042 | SEQ ID NO: 63 | Chimeric: RSPO2Fu1_32-76-RSPO4Fu2_72-136- |
| | | RSPO1_144-263 |
| #046 | SEQ ID NO: 64 | hRSPO1 21-263-short-Linker-Peptide with high affinity for |
| | | Albumin |
| #047 | SEQ ID NO: 27 | (Hybrid/Variant) Derived from human Rspo1 32-263 with 4 |
| | | additional amino acid substitutions E49K; V50D; D68G; |
| | | D85G from corresponding alignment in hRspo2 |
| #048 | SEQ ID NO: 65 | hRspo1 32-263 Mutant in Fu1: |
| | | E49K; V50D; D68G; D85G; E45L; K55R; N88A |
| #049 | SEQ ID NO: 66 | hRspo1 32-263 Mutant in Fu2: H108K; N109D |
| #050 | SEQ ID NO: 67 | hRspo1 32-263 Mutant in Fu1 and Fu2: |
| | | E49K; V50D; D68G; D85G; H108K |
| #051 | SEQ ID NO: 28 | (Hybrid/variant) hRspo1 32-263 with 9 additional amino |
| | | acid substitutions E45L; E49K; V50D; K55R; D68G; D85G; |
| | | N88A; H108K, N109D |
| #052 | SEQ ID NO: 68 | hRspo1 32-263 Mutants in Fu1: E49K; V50D |
| #053 | SEQ ID NO: 69 | hRspo1 32-263 Mutants in Fu1: D68G; D85G |
| #054 | SEQ ID NO: 70 | hRspo1 32-263 Mutant in Fu2: H108K |
| #055 | SEQ ID NO: 71 | hRspo1 32-263 Mutant in Fu1: L46S; S57Q; I62F; L63F; |
| | | P77H; F84Y; I95A |
| #056 | SEQ ID NO: 72 | hRspo1 32-263 Mutant in Fu 2: E116V; L118F; P127D; |
| | | A128E; S133F; A136L; G138E; S143V |
| #057 | SEQ ID NO: 73 | hRspo1 32-263 Mutant in Fu1 and Fu2: L46S; S57Q; I62F; |
| | | L63F; P77H; F84Y; I95A; E116V; L118F; P127D; A128E; |
| | | S133F; A136L; G138E; S143V |
| #063 | SEQ ID NO: 42 | hRspo1 21-263-Linker- Fc |
| #064 | SEQ ID NO: 43 | Fc-Linker-hRspo1-21-263 |
| #068 | SEQ ID NO: 74 | hRspo1 32-263 Mutant in Fu2: H108R |
| #069 | SEQ ID NO: 75 | hRspo1 32-263 Mutant in Fu2: N109D |
| #071 | SEQ ID NO: 76 | hRspo1 32-263 Mutant in the BR Domain: L227A |
| #072 | SEQ ID NO: 77 | hRspo1 32-263 Mutant in the BR Domain: T253A |
| #073 | SEQ ID NO: 78 | hRspo1 32-263 Mutant in the BR Domain: T253A; T258A; |
| | | S259A; S268A if Linker at C-terminal is present |
| #074 | SEQ ID NO: 79 | hRspo1 32-263 Mutant in the BR Domain: G252T |
| #075 | SEQ ID NO: 80 | hRspo1 32-263 Mutant in the BR Domain: L257S |
| #076 | SEQ ID NO: 81 | hRspo1 32-263 Mutant in the BR Domain: A260T |
| #077 | SEQ ID NO: 82 | hRspo1 32-263 Mutant in the BR Domain: A263T |
| #078 | SEQ ID NO: 83 | hRspo1 32-263 Mutant in the BR Domain: |
| | | G252T; L257S; A260T; A263T |
| #079 | SEQ ID NO: 84 | hRspo1 32-263 Mutant in the BR Domain: T253E |
| #080 | SEQ ID NO: 85 | hRspo1 32-263 Mutant in the BR Domain: T253E, T258E, |
| | | S259E, S268E |
| #081 | SEQ ID NO: 86 | hRspo1 32-263 Mutant in the BR Domain: N253GTN256 |
| | | (Native sequence is T253 VGP256) |
| #082 | — | Rspo1 with N-Terminal deletion 32-263 and R66A single |
| | | mutation |
| #084 | — | Protein #26 with Fu1 mutation R66A |
| #086 | — | Protein #32 with Fu1 mutation R66A |
| #088 | — | Protein #37 with Fu1 mutation R66A |
| #093 | — | hRSPO1-32-263-Q71A Mutation in Fu1 |
| #094 | SEQ ID NO: 87 | hRSPO1 21-263-Long Linker-Peptide with high affinity for |
| | | Albumin |
| #095 | SEQ ID NO: 88 | Peptide with high affinity for Albumin-Linker-hRSPO1 21- |
| | | 263 |
| #096 | — | hRspo1 21-263 Q71A mutation in Fu1 |
| #106 | — | Mutant #051 with additional mutation R66A |
| #108 | — | Mutant in the Fu1 and Fu2 Domains: E49K, V50D, D68G, |
| | | D85G, H108K, N109D |
| #109 | — | Mutant in the Fu1 and Fu2 Domains: E49K, V50D, D68G, |
| | | D85G, N109D |
| #110 | — | Mutant in Fu2 Domain: H108K, N109E |
| #111 | — | Mutant in the Fu1 and Fu2 Domains: E49K, V50D, D68G, |
| | | D85G, H108K, N109E |
| #112 | — | Mutant in the Fu1 and Fu2 Domains: E49K, V50D, D68G, |
| | | D85G, N109E |
| #113 | — | #008 with Fc at The C terminal |
| #114 | — | #082 with Fc at the C terminal |
| #115 | — | #106 with Fc at the C terminal |
| #116 | SEQ ID NO: 94 | #116 hRspo1 32-244 |
| #121 | SEQ ID NO: 92 | #049 with Fc at the N-terminal |
| #155 | SEQ ID NO: 93 | #049 with Fc at the C-terminal |
| #150 | SEQ ID NO: 95 | #116 hRspo1 32-244 with Fc at the C-terminal |
| #195 | SEQ ID NO: 96 | Full Length Variant #195 w/o SP [(hRspo1 32-263) with Fc |
| | | fragment at N-Terminal] |
| | SEQ ID NO: 97 | Full Length Variant #195 with SP [(hRspo1 32-263) with |
| | | Fc fragment at N-Terminal] |
| | SEQ ID NO: 98 | Full Length Variant #121 w/o SP (hRspo1 32-263 with |
| | | Mutant in Fu2: H108K; N109D) with Fc fragment at N- |
| | | Terminal |
| #199 | SEQ ID NO: 99 | hRspo1 32-263-SA-Fc |
| #200 | SEQ ID NO: 100 | hRspo1 32-263 with Mutant in Fu2: H108K; N109D-SA-Fc |
| #198 | SEQ ID NO: 105 | hRspo1 32-263-GGGGGGSGGGGSA-Fc |
| #194 | SEQ ID NO: 106 | hRspo1 32-263 with Mutant in Fu2: H108K; N109D- |
| | | GGGGGGSGGGGSA-Fc |
| #193 | SEQ ID NO: 107 | hRspo1 32-263 with Mutant in Fu2: H108K; N109D- |
| | | GGGSGGGGSA-Fc |
| #192 | SEQ ID NO: 108 | hRspo1 32-263 with Mutant in Fu2: H108K; N109D- |
| | | SGGGGSA-Fc |
| #190 | SEQ ID NO: 109 | hRspo1 21-263-GGGGGGSGGGGSA-Fc |
| #189 | SEQ ID NO: 110 | hRspo1 21-263-GGGSGGGGSA-Fc |
| #188 | SEQ ID NO: 111 | hRspo1 21-263-SGGGGSA-Fc |

Conservative Modifications and Functional Equivalents

Additional functional equivalents of the Variants as described in the previous sections A-F with similar advantageous properties of native R-spondin 1 proteins, or functional equivalents of Rspo3 polypeptide as described in the previous section G with similar advantageous properties of native human R-spondin 3 protein, can be further identified by screening candidate molecules and testing whether such candidate molecules have maintained the desired functional properties when compared to either Rspo1, Rspo3 or to any of the specific Variants as disclosed herein (in particular those preferred Variants referred to in the above Table 1).

In specific embodiments, a functional equivalent of the Variants binds to LGR4 receptor with at least the same affinity as one of the preferred Variants as disclosed herein.

In specific embodiments, said functional equivalent of a specific Variant as disclosed herein exhibits at least 90%, 100% or more, of one or more of the following activities relative to the Rspo1 protein of SEQ ID NO:41:
(i) Binding affinity to LGR4 receptor, for example as determined by SPR assay;
(ii) Induction of the proliferation of functional beta-cells, for example as determined in an in vitro Min6 beta-cell proliferation assay;
(iii) Induction of the proliferation of functional beta-cells, for example as determined in an in vivo beta-cell proliferation assay.

In specific embodiments, said functional equivalent of a Rspo3 polypeptide as disclosed herein exhibits at least 90%, 100% or more, of one or more of the following activities relative to the Rspo3 protein of SEQ ID NO:3:
(i) Binding affinity to LGR4 receptor, for example as determined by SPR or ELISA assay;
(ii) Binding affinity to ZNRF3 receptor, for example as determined by SPR or ELISA assay;
(iii) Induction of the proliferation of functional beta-cells, for example as determined in an in vitro Min6 beta-cell proliferation assay;
(iv) Induction of the proliferation of functional beta-cells, for example as determined in an in vivo beta-cell proliferation assay.

Further details of the assays and conditions for use in determining the activities are disclosed in the experimental part below.

In specific embodiments, said functional equivalent of a specific Variant exhibits at least 90%, and more preferably, 100% or more of the above desired activities relative to one of the corresponding preferred Variant as disclosed in Table 1.

Functional equivalents of the Variants as disclosed in the previous Sections may be obtained typically by amino acid substitution, deletion or insertion as compared to a corresponding Variant in non-essential residues. In a particular embodiment, said functional equivalent differs from the corresponding Variant, through only amino acid substitutions, with natural or non-natural amino acids, preferably only 1, 2, 3, 4, 5, 6, 7, 8, 9 or 10 amino acid substitutions with natural amino acids, in particular as compared to one of the Variants as described in Table 1.

In other embodiments, said functional equivalent is a polypeptide having 95% identity to at least one of SEQ ID NO:22-28, and wherein said polypeptide comprises a FU1 and FU2 domain which is 100% identical to the FU1 and FU2 domains of at least one of SEQ ID NO:22-28.

In more specific embodiments, the amino acid sequence of said functional equivalent may differ from the Variants as disclosed herein, in particular in Table 1, through mostly conservative amino acid substitutions; for instance at least 10, such as at least 9, 8, 7, 6, 5, 4, 3, 2 or 1 of the substitutions in the variant are conservative amino acid residue replacements.

In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also may be substantially retained in a variant mutant polypeptide as compared to a parent Variant of Table 1.

In specific embodiments, a functional equivalent of a Variant comprises a polypeptide which is identical to any one of SEQ ID NOs: 22-28 (the variants of Table 1), except for 1, 2 or 3 amino acid residues which have been replaced by another natural amino acid, preferably by conservative amino acid substitutions as defined above.

In addition, the person skilled in the art will appreciate that the conserved residues among various species or among the different members of Rspo family may be important to maintain the proper structure and therefore, the skilled person may refrain from mutating such amino acid positions. For example, conserved residues among Rspo1, Rspo2, Rspo3 and Rspo4 are shown in FIG. 1 in particular and may preferably not be mutated, unless otherwise specified in the present disclosure.

Alternatively, at many sites, one or two or more amino acids positions show conservative variations among species variants, and/or among other members of Rspo family, such as Rspo2, Rpo3 and Rspo4. One of skill in the art would understand that some of such conservative substitutions may likely not adversely affect the function of Rspo1 and may therefore be mutated as compare to native R-spondin 1 with such conservative variations.

In specific embodiments, the Variants of the Disclosure comprises a FU2 domain having the following amino acid residue which have not been mutated as compared to the native R-spondin protein: F106, H108, F110, N109, E116, L118, P127, A128, S133, A136, G138, S143 in human Rspo1 of SEQ ID NO:1.

In specific embodiments, the Variants of the Disclosure comprises a BR domain which do not have amino acid changes as compared to the native BR domain at one or more of the following positions: T253, L257, T258, S259, A260 or A263, in human Rspo1 of SEQ ID NO:1, or at the corresponding residues in human Rspo2 of SEQ ID NO:2, human Rspo3 of SEQ ID NO:3 or human Rspo4 of SEQ ID NO:4, typically said BR domain is 100% identical to SEQ ID NO: 8, SEQ ID NO:12, SEQ ID NO:16, or SEQ ID NO:20.

Fusion Proteins of the Disclosure

A variety of polypeptides other than R-spondin 1 polypeptides can be fused to the Variants of the disclosure or their functional equivalents as described above (in particular the Variants of Table 1), for a variety of purposes such as, for example, to increase in vivo half-life of the protein, to facilitate identification, isolation and/or purification of the protein, to increase the activity of the protein, and to promote oligomerization of the protein.

Many polypeptides can facilitate identification and/or purification of a recombinant fusion protein of which they are a part. Examples include polyarginine, polyhistidine. Polypeptides comprising polyarginine allow effective purification by ion exchange chromatography. For example, in specific embodiments, a Variant of the Disclosure (and more specifically any of the preferred Variants as disclosed in Table 1) includes a polyhistidine C-terminal tag and, optionally a peptide linker, for example the polypeptide of SEQ ID NO: 90 (GGGGSEPEAHHHHHH).

In a specific embodiment, a polypeptide that comprises an Fc region of an antibody, optionally an IgG antibody, or a substantially similar protein, can be fused to a Variant of the Disclosure (typically one the Variants disclosed in Table 1) or functional equivalents thereof, directly, or optionally via a peptidic linker, thereby forming an Fc fusion protein of the present disclosure. An example of such Fc region is an IgG4 Fc fragment or a derivative thereof, typically the polypeptide of SEQ ID NO:29. In another particular embodiment, the Fc region can be any of the IgG1, IgG2, IgG3, IgG4, IgA, IgA, IgD, IgE or IgM isotype.

Said Fc fusion protein can be a Fc-homodimer ((Rspo1 variant-Fc)₂) and/or monomeric Fc (also named monovalent Fc fusion protein) (Rspo1 variant-(Fc)₂).

In a specific embodiment, the disclosure also relates to a Fc fusion protein wherein the Fc fragment is fused to the Rspo1 protein of SEQ ID NO: 66 (either at the C-terminal or N-terminal, optionally via a peptidic linker) or variant thereof, and more specifically the Fc fragment comprises SEQ ID NO: 29.

In another specific embodiment, the disclosure also relates to a Fc fusion protein wherein the Fc fragment is fused to the Rspo1 protein of SEQ ID NO: 24 (either at the C-terminal or N-terminal, optionally via a peptidic linker) or variant thereof, and more specifically the Fc fragment comprises SEQ ID NO: 29.

In another specific embodiment, the disclosure also relates to a Fc fusion protein wherein the Fc fragment is fused to the Rspo1 protein of SEQ ID NO: 94 (either at the C-terminal or N-terminal, optionally via a peptidic linker) or variant thereof, and more specifically the Fc fragment comprises SEQ ID NO: 29.

In a specific embodiment, an Fc polypeptide of SEQ ID NO:29 is fused directly or indirectly via a peptidic linker at the C-terminal end of one of the preferred Variants of Table 1.

The inventors surprisingly showed that the fusion of an Fc polypeptide directly or indirectly via a peptidic linker at the N-terminal end of a Variant of the present disclosure allow greater recovery of the desired Fc fusion protein.

Thus, in a preferred embodiment, an Fc polypeptide of SEQ ID NO: 29 is fused directly or indirectly via a peptidic linker at the N-terminal end of one of the preferred Variants of Table 1, preferably of variant of an amino acid sequence of SEQ ID NO: 41, 24, 66 or 94.

In a preferred embodiment, the disclosure also relates to a Fc fusion protein wherein the Fc fragment is fused to the Rspo1 protein of SEQ ID NO: 41 at N-terminal, optionally via a peptidic linker, and more specifically the Fc fragment comprises SEQ ID NO: 29, preferably said Fc fusion protein comprises or consists of an amino acid sequence of SEQ ID NO: 43.

In a specific embodiment, the disclosure also relates to a Fc fusion protein wherein the Fc fragment is fused to the Rspo1 protein of SEQ ID NO: 66 at N-terminal, optionally via a peptidic linker, and more specifically the Fc fragment comprises SEQ ID NO: 29, preferably said Fc fusion protein comprises or consists of an amino acid sequence of SEQ ID NO: 92 or 98.

In another specific embodiment, the disclosure also relates to a Fc fusion protein wherein the Fc fragment is fused to the Rspo1 protein of SEQ ID NO: 24 at N-terminal, optionally via a peptidic linker, and more specifically the Fc fragment comprises SEQ ID NO: 29, preferably said Fc fusion protein comprises or consists of an amino acid sequence of SEQ ID NO: 96 or 97.

The disclosure also relates to a Fc fusion protein wherein the Fc fragment is fused to the Rspo1 protein of SEQ ID NO:1 (either at the C-terminal or N-terminal, optionally via a peptidic linker), and more specifically the Fc fragment comprises an amino acid sequence of SEQ ID NO: 29.

In specific embodiments, the Variants of the Disclosure is an Fc fusion protein comprising or consisting of SEQ ID NO:42, SEQ ID NO:43, SEQ ID NO: 92, SEQ ID NO: 93, SEQ ID NO: 98, SEQ ID NO: 95, SEQ ID NO: 96 or SEQ ID NO: 97 (corresponding to Variants #063, #064, #121, #155 (with or without signal peptide), #150 or #195 (without or with signal peptide), respectively).

In a preferred embodiment, said Fc fusion protein comprises a peptide signal of SEQ ID NO: 99, preferably said Fc fusion protein comprises or consists of an amino acid sequence of SEQ ID NO: 97 or 92.

A fusion protein may also comprise one or more peptide linkers. Generally, a peptide linker is a stretch of amino acids that serves to link plural polypeptides to form multimers and provides the flexibility or rigidity required for the desired function of the linked portions of the protein. Typically, a peptide linker is between about 1 and 30 amino acids in length. Examples of peptide linkers include, but are not limited to, -Gly-Gly-, GGGGS (SEQ ID NO: 46), (GGGGS)n (wherein n is between 1-8, typically 3 or 4), or SA. Linking moieties are described, for example, in Huston, J. S., et al., Proc. Natl. Acad. Sci. 85: 5879-83 (1988), Whitlow, M., et al., Protein Engineering 6: 989-95 (1993), Newton, D. L., et al., Biochemistry 35: 545-53 (1996), and U.S. Pat. Nos. 4,751,180 and 4,935,233.

In a particular embodiment, the inventors showed that a linker optimization is required to generate active Fc fusion protein.

Preferred peptide linkers which may be used between the Fc part and the R-spondin part of the Fc fusion protein include for example the linker of (GGGGGGSGGGGSGGGGSA) (SEQ ID NO:44), (GGGGSGGGGSGGGGGG) (SEQ ID NO:45), GGGGS (SEQ ID NO: 46), (GGGGGGSGGGGSA) (SEQ ID NO: 102), (GGGSGGGGSA) (SEQ ID NO: 103), (SGGGGSA) (SEQ ID NO: 104), GG or SA.

The inventors have tested different linkers (GGGGGGSGGGGSA) (SEQ ID NO: 102), (GGGSGGGGSA) (SEQ ID NO: 103), (SGGGGSA) (SEQ ID NO: 104) and SA and showed that a short linker such as SA allows greater recovery of the desired Fc fusion protein.

In certain embodiments, the Fc fusion is fused indirectly via a peptide linker at the C-terminal end of the Variants of the Disclosure or their functional equivalents said peptidic linker consists of the amino acid sequence selected from the group consisting of SEQ ID NO: 44, 45, 46, 102, 103, 104, GG or SA.

In other embodiments, the Fc fusion is fused indirectly via a peptide linker at the N-terminal end of the Variants of the Disclosure or their functional equivalents, said peptidic linker consists of the amino acid sequence selected from the group consisting of SEQ ID NO: 44, 45, 46, 102, 103, 104, GG or SA.

In a preferred embodiment, the present disclosure relates to a Fc fusion protein wherein the Fc fragment of SEQ ID NO: 29 is fused to the Rspo1 protein of SEQ ID NO: 24 at the C-terminal via a peptide linker SA, preferably said Fc fusion protein comprises or consists of an amino acid sequence of SEQ ID NO: 100.

In another particular embodiment, the present disclosure relates to a Fc fusion protein wherein the Fc fragment of SEQ ID NO: 29 is fused to the Rspo1 protein of SEQ ID NO: 24 at the C-terminal via a peptide linker of SEQ ID NO: 102, preferably said Fc fusion protein comprises or consists of an amino acid sequence of SEQ ID NO: 105.

In a preferred embodiment, the present disclosure relates to a Fc fusion protein wherein the Fc fragment of SEQ ID NO: 29 is fused to the Rspo1 protein of SEQ ID NO: 66 at the C-terminal via a peptide linker SA, preferably said Fc fusion protein comprises or consists of an amino acid sequence of SEQ ID NO: 101.

In another particular embodiment, the present disclosure relates to a Fc fusion protein wherein the Fc fragment of SEQ ID NO: 29 is fused to the Rspo1 protein of SEQ ID NO: 66 at the C-terminal via a peptide linker of SEQ ID NO: 102, preferably said Fc fusion protein comprises or consists of an amino acid sequence of SEQ ID NO: 106.

In another particular embodiment, the present disclosure relates to a Fc fusion protein wherein the Fc fragment of SEQ ID NO: 29 is fused to the Rspo1 protein of SEQ ID NO: 66 at the C-terminal via a peptide linker of SEQ ID NO: 103, preferably said Fc fusion protein comprises or consists of an amino acid sequence of SEQ ID NO: 107.

In another particular, the present disclosure relates to a Fc fusion protein wherein the Fc fragment of SEQ ID NO: 29 is fused to the Rspo1 protein of SEQ ID NO: 66 at the C-terminal via a peptide linker of SEQ ID NO: 104, preferably said Fc fusion protein comprises or consists of an amino acid sequence of SEQ ID NO: 108.

In another particular, the present disclosure relates to a Fc fusion protein wherein the Fc fragment of SEQ ID NO: 29 is fused to the Rspo1 protein of SEQ ID NO: 41 at the C-terminal via a peptide linker of SEQ ID NO: 102, preferably said Fc fusion protein comprises or consists of an amino acid sequence of SEQ ID NO: 109.

In a preferred embodiment, the present disclosure relates to a Fc fusion protein wherein the Fc fragment of SEQ ID NO: 29 is fused to the Rspo1 protein of SEQ ID NO: 41 at the C-terminal via a peptide linker of SEQ ID NO: 103, preferably said Fc fusion protein comprises or consists of an amino acid sequence of SEQ ID NO: 110.

In a preferred embodiment, the present disclosure relates to a Fc fusion protein wherein the Fc fragment of SEQ ID NO: 29 is fused to the Rspo1 protein of SEQ ID NO: 41 at the C-terminal via a peptide linker of SEQ ID NO: 104, preferably said Fc fusion protein comprises or consists of an amino acid sequence of SEQ ID NO: 111.

Another modification of the Variants or their functional equivalents that is contemplated by the present disclosure is a conjugate or a protein fusion of at least the Variants or equivalent to a serum protein, such as human serum albumin or a fragment thereof to increase half-life of the resulting molecule. Such approach is for example described in Ballance et al. EP 0 322 094.

Another possibility is a fusion protein of the disclosure including proteins capable of binding to serum proteins, such as proteins binding to human serum albumin (i.e. anti-HSA fusion protein) to increase half-life of the resulting molecule, including for example anti-HSA binding moieties derived from Fab or nanobody that binds to HSA or any other domain type structures such as darpin, nanofitin, fynomer and the like. Such approach is for example described in Journal of Controlled Release 301 (2019) 176-189; BioDrugs (2015) 29:215-239; J Pharmacol Exp Ther 370:703-714, September 2019; Biodrugs 2009; 23 (2): 93-109. Examples of such fusion proteins includes the polypeptides of SEQ ID NO: 64, NO:87 or NO:88.

In other embodiments, a fusion protein of the disclosure includes one of the peptides binding to albumin as disclosed in Dennis et al 2002 (Journal of Biological Chemistry, 2002, Vol 277, No. 38, pp 35035-35043), and in particular a peptide comprising the core sequence of SEQ ID NO:91 (DICLPRWGCLW).

Examples of such variants are the polypeptide of SEQ ID NO: 64 (Variant #046) or the polypeptide of SEQ ID NO: 87 (Variant #094) or SEQ ID NO: 88 (Variant #095).

A recombinant fusion protein of the disclosure can comprise a polypeptide comprising a leucine zipper or other multimerization motifs. Among known leucine zipper sequences are sequences that promote dimerization and sequences that promote trimerization. See e.g. Landschulz et al. (1988), Science 240: 1759-64). Leucine zippers comprise a repetitive heptad repeat, often with four or five leucine residues interspersed with other amino acids. Use and preparation of leucine zippers are well known in the art.

Another modification of the Variants or functional equivalents disclosed herein that is contemplated by the present disclosure is pegylation or hesylation or related technologies such as PASylation.

More generally, the Variants or their functional equivalents may be conjugated with biodegradable bulking agents, including natural and semi-synthetic polysaccharides, including O- and N-linked oligosaccharides, dextran, hydroxyethylstarch (HES), polysialic acid and hyaluronic acid, as well as unstructured protein polymers such as homo-amino acid polymers, elastin-like polypeptides, XTEN and PAS

For example, a Variant of the disclosure or their functional equivalent can be pegylated to, for example, increase the biological (e.g., serum) half-life of the antibody. To pegylate, the recombinant protein is reacted with polyethylene glycol (PEG), such as a reactive ester or aldehyde derivative of PEG, under conditions in which one or more PEG groups become attached to the recombinant protein. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins are known in the art and can be applied to the proteins of the disclosure. See for example, Jevsevar et al 2010 Biotechnol J. 5(1): 113-28, or Turecek et al 2016 J Pharm Sci 2016 105(2): 460-375. Hence, in specific embodiments, the Rspo1 protein of the disclosure is pegylated.

Another modification of the Variants or their functional equivalents that is contemplated by the present disclosure is PASylation. See for example: Protein Engineering, Design & Selection vol. 26 no. 8 pp. 489-501, 2013. Hence, in specific embodiments, the Rspo1 protein of the disclosure is PASylated.

Xten technology is for example described in are reviewed for example in Nature Biotechnology volume 27 number 12 2009: 1186-1192.

Nucleic Acid Molecules Encoding the Proteins of the Disclosure

Also disclosed herein are the nucleic acid molecules that encode the Variants of the Disclosure or their functional equivalents.

The tables 10 and 11 in the Examples below provide specific examples of nucleotide sequence encoding certain amino acid sequences of the Variants disclosed herein.

Examples of nucleotide sequences are those encoding the amino acid sequences of any one of the examples as described in the above Table 1, in particular encoding any one of SEQ ID NO:22-28, SEQ ID NO:42-43, SEQ ID NO:50-89, SEQ ID NO: 92-98, SEQ ID NO: 100-101, and SEQ ID NO: 105-111. the nucleic acid sequences being easily derived from the Table 9, and using the genetic code and, optionally taking into account the codon bias depending on the host cell species.

The present disclosure also pertains to nucleic acid molecules that derive from the latter sequences having been optimized for protein expression in mammalian cells, for example, mammalian Chinese Hamster Ovary (CHO) or human HEK293 cell lines.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. A nucleic acid of the disclosure can be, for example, DNA or RNA and may or may not contain intronic sequences. In an embodiment, the nucleic acid may be present in a vector such as a phage display vector, or in a recombinant plasmid vector.

Nucleic acids of the disclosure can be obtained using standard molecular biology techniques. Once DNA fragments encoding a Variant of the Disclosure are obtained, these DNA fragments can be further manipulated by standard recombinant DNA techniques. In these manipulations, a Variant-encoding DNA fragment may be operatively linked to another DNA molecule, or to a fragment encoding another protein, such as an antibody constant region (Fc region) or a flexible linker. Examples of nucleotide sequences further include nucleotide sequences encoding a recombinant fusion protein, in particular an Fc fusion protein comprising coding sequences of any one of the amino acid sequence SEQ ID NO 1 or variant thereof operatively linked with a coding sequence of an Fc region (e.g., SEQ ID NO: 29), for example SEQ ID NO:42, SEQ ID NO:43, SEQ D NO: 92, SEQ ID NO: 93, SEQ ID NO: 95-98, SEQ ID NO: 100, SEQ ID NO: 101 or SEQ ID NO: 105-111.

The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

Generation of Transfectomas Producing the Variants or Fusion Proteins of the Disclosure

The Variants of the Disclosure or their functional equivalents, and/or related fusion proteins can be produced in a host cell transfectoma using, for example, a combination of recombinant DNA techniques and gene transfection methods as is well known in the art.

For example, to express the Variants or related fusion proteins of the Disclosure, or corresponding functional equivalents thereof, DNAs encoding partial or full-length recombinant proteins can be obtained by standard molecular biology or biochemistry techniques (e.g., DNA chemical synthesis, PCR amplification or cDNA cloning) and the DNAs can be inserted into expression vectors such that the genes are operatively linked to transcriptional and translational control sequences.

In this context, the term “operatively linked” is intended to mean that an antibody gene is ligated into a vector such that transcriptional and translational control sequences within the vector serve their intended function of regulating the transcription and translation of the recombinant protein. The expression vector and expression control sequences are chosen to be compatible with the expression host cell used. The protein encoding genes are inserted into the expression vector by standard methods (e.g., ligation of complementary restriction sites on the antibody gene fragment and vector, or blunt end ligation if no restriction sites are present).

The recombinant expression vector can encode a signal peptide that facilitates secretion of the recombinant protein from a host cell. The Variant encoding gene can be cloned into the vector such that the signal peptide is linked in frame to the amino terminus of the recombinant protein. The signal peptide can be the native signal peptide of Rspo1, Rspo2, Rspo3 or Rspo4 or a heterologous signal peptide (i.e., a signal peptide from a non-Rspondin protein).

In addition to the Variant encoding genes, the recombinant expression vectors disclosed herein carry regulatory sequences that control the expression of the recombinant protein in a host cell. The term “regulatory sequence” is intended to include promoters, enhancers and other expression control elements (e.g., polyadenylation signals) that control the transcription or translation of the protein encoding genes. It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1.

In addition to the Variant encoding genes and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr− host cells with methotrexate selection/amplification) and the neo gene (for G418 selection).

For expression of the Variant of the Disclosure or related fusion proteins, the expression vector(s) encoding the recombinant protein is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the proteins of the present disclosure in either prokaryotic or eukaryotic host cells. Expression of proteins in eukaryotic cells, for example mammalian host cells, yeast or filamentous fungi, is discussed because such eukaryotic cells, and in particular mammalian cells, are more likely than prokaryotic cells to assemble and secrete a property folded and functional recombinant protein.

In one specific embodiment, a cloning or expression vector according to the disclosure comprises one of the coding sequences of the Variants as described in Table 1 (typically of SEQ ID Nos 22-28 or SEQ ID NO:50-89) or of the fusion proteins (typically SEQ ID NO:42, 43, 92-98, SEQ ID NO: 100, 101 or SEQ ID NO: 105-111), operatively linked to suitable promoter sequences.

Mammalian host cells for expressing the recombinant proteins of the disclosure include Chinese Hamster Ovary (CHO cells), including dhfr− CHO cells (described in Uriaub and Chasin, 1980) used with a DHFR selectable marker (as described in Kaufman and Sharp, 1982), CHOK1 dhfr+ cell lines, NSO myeloma cells, COS cells, HEK293 cells and SP2 cells. When recombinant expression vectors encoding antibody genes are introduced into mammalian host cells, the recombinant proteins are produced by culturing the host cells for a period of time sufficient for expression of the recombinant proteins in the host cells and, optionally, secretion of the proteins into the culture medium in which the host cells are grown.

The Variants or related fusion proteins of the Disclosure can be recovered and purified for example from the culture medium after their secretion using standard protein purification methods.

In one specific embodiment, the host cell of the disclosure is a host cell transfected with an expression vector having the coding sequences suitable for the expression of one of the Variant comprising any one of SEQ ID NO:22-28, SEQ ID NO:42-43, SEQ ID NO:50-89 and SEQ ID NO: 92-98, SEQ ID NO: 100-101 or SEQ ID NO: 105-111, respectively, operatively linked to suitable promoter sequences.

The latter host cells may then be further cultured under suitable conditions for the expression and production of a recombinant Variant or related fusion proteins of the Disclosure.

Pharmaceutical Compositions

In another aspect, the present disclosure provides a composition, e.g., a pharmaceutical composition, containing a Variant or related fusion proteins of the Disclosure or their functional equivalents. Such compositions may include one or a combination of (e.g., two or more different) recombinant Variants or related fusion proteins, as described above.

For example, said pharmaceutical composition comprises a recombinant protein comprising any one of the preferred Variants disclosed in Table 1, typically comprising any polypeptide of SEQ ID NO:22-28 and SEQ ID NO:50-89, or a fusion protein, for example of SEQ ID Nos: 42-43, SEQ ID NO: 92-98, SEQ ID NO: 100-101 or SEQ ID NO: 105-111 or a functional equivalent thereof, formulated together with a pharmaceutically acceptable carrier.

Pharmaceutical compositions disclosed herein can also be administered in combination therapy, i.e., combined with other agents. For example, the combination therapy can include a Variant of the Disclosure, for example a recombinant protein comprising a polypeptide of any one of SEQ ID NO:22-28, SEQ ID NO:42-43, SEQ ID NO:50-89, SEQ ID NO: 92-98, SEQ ID NO: 100-101 and SEQ ID NO: 105-111 or a functional equivalent thereof, combined with at least one anti-inflammatory, or another anti-diabetic agent. Examples of therapeutic agents that can be used in combination therapy are described in greater detail below in the section on uses of the recombinant Variants or related fusion proteins of the Disclosure.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for a parenteral, intranasal, intravenous, intramuscular, subcutaneous or intraocular administration (e.g., by injection or infusion).

In one embodiment, the carrier should be suitable for subcutaneous route or intravenous injection. Depending on the route of administration, the active compound, i.e., the Variants of the Disclosure, may be coated in a material to protect the compound from the action of acids and other natural conditions that may inactivate the compound.

Sterile phosphate-buffered saline is one example of a pharmaceutically acceptable carrier. Other suitable carriers are well-known to those in the art. (Remington and Gennaro, 1995). Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for oral, intranasal, sublingual, subcutaneous, intramuscular, intravenous, transdermal, parenteral, or rectal administration and the like. The Variants of the Disclosure as an active principle, alone or in combination with another active principle, can be administered in a unit administration form, as a mixture with conventional pharmaceutical supports, to animals and human beings.

Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, a therapeutically effective amount of the Variants of the Disclosure may be dissolved or dispersed in a pharmaceutically acceptable carrier or aqueous medium.

The pharmaceutical forms suitable for injectable use may include sterile aqueous solutions or dispersions; formulations including sesame oil, peanut oil or aqueous propylene glycol; and sterile powders or lyophilisates for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

Variants or related fusion proteins of the Disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds, i.e. the Rspo1 proteins, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Variants or related fusion proteins of the Disclosure or their functional equivalents may be formulated within a therapeutic mixture to comprise about 0.01 mg-1000 mg/kg or 1 mg-100 mg/kg. Multiple doses can also be administered.

Suitable formulation for solution for infusion or subcutaneous injection of the recombinant proteins have been described in the art and for example are reviewed in Advances in Protein Chemistry and Structural Biology Volume 112, 2018, Pages 1-59 Therapeutic Proteins and Peptides Chapter One—Rational Design of Liquid Formulations of Proteins: Mark C. Manning, Jun Liu, Tiansheng Li, Ryan E. Holcomb.

Uses and Methods of the Variants or Related Fusion Proteins of the Disclosure

The Variants or related fusion proteins of the Disclosure or their functional equivalents have in vitro and in vivo utilities. For example, these recombinant proteins can be administered to cells in culture, e.g. in vitro, ex vivo or in vivo, or in a subject, e.g., in vivo, to treat, or prevent a variety of disorders.

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

In particular, with reference to the treatment of a diabetes, more specifically diabetes type 1, the term “treatment” may refer to the inhibition of the loss of pancreatic beta-cells, and/or the increase of the mass of pancreatic beta-cells, in particular functional insulin secreting beta-cells in said subject, and/or improvement of glycemia control, in particular in patients having loss of pancreatic beta-cells and/or islets of Langerhans due to a disease, for example diabetes type 1.

The Variants or related fusion proteins of the Disclosure or their functional equivalents can induce the proliferation of pancreatic beta-cells in vivo and reconstitute functional insulin-secreting islets of Langerhans, and thereby may be used to treat diabetic patients, or patients in need of functional insulin-secreting beta-cells, or patients with disorders associated with hyperglycemia, or patients with deficient glucose stimulated insulin secretion.

As used herein, the terms “diabetes” generally refers to any conditions or disorders resulting in insulin shortage or resistance to its action Examples of diabetes include, but are not limited to, type 1, type 2, gestational, and Latent autoimmune diabetes in adults (LADA).

Accordingly, the disclosure relates to a method for treating one of the disorders disclosed above, in a subject in need thereof, said method comprising administering to said subject a therapeutically efficient amount of Variant or related fusion protein of the Disclosure or a functional equivalent as disclosed above, typically, a recombinant protein comprising a polypeptide of any one of SEQ ID NO:22-28, SEQ ID NO:42-43, SEQ ID NO:50-89, SEQ ID NO: 92-98, SEQ ID NO: 100-101 and SEQ ID NO: 105-111, or a functional equivalent thereof.

In certain embodiments, said subject has been selected among patient having low Rspo1 gene expression.

The Variant or related fusion protein, or a functional equivalent thereof, for use as disclosed above may be administered as the sole active ingredient or in conjunction with, e.g. as an adjuvant to or in combination to, other drugs e.g. cytokines, anti-viral, anti-inflammatory agents, anti-diabetic or hypoglycemiant agents, cell therapy product (e.g beta-cell composition) and immune modulatory drugs, e.g. for the treatment or prevention of diseases mentioned above.

For example, the Variant or related fusion protein, or a functional equivalent thereof, for use as disclosed above may be used in combination with cell therapy, in particular p cell therapy.

As used herein, the term “cell therapy” refers to a therapy comprising the in vivo administration of at least a therapeutically efficient amount of a cell composition to a subject in need thereof. The cells administered to the patient may be allogenic or autologous. The term “s cell therapy” refers to a cell therapy wherein the cell composition includes, as the active principle, s cells, in particular insulin secreting beta-cells. Such beta-cells may be produced using the Rspo1 proteins in an in vitro method as described hereafter.

In a particular embodiment, a Rspo1 protein, the variant or related fusion protein or a functional equivalent thereof for use to treat diabetic patients, or patients in need of functional insulin-secreting beta-cells, or patients with disorders associated with hyperglycemia, or patients with deficient glucose stimulated insulin secretion is administered in combination with (e.g., before, simultaneously or following) a beta-cell composition, in particular stem cell-derived beta-cell composition.

In a particular embodiment, said beta-cell is isolated from a live donor, a cadaveric donor.

In another particular embodiment, said beta-cell is a stem cell-derived beta-cell. Stem cell-derived beta-cell refers to insulin secreting beta-cell obtained by the differentiation of pluripotent stem cells, in particular induced pluripotent stem cell or human embryonic stem cells. In an embodiment, when stems cells are human stem cells, said human stem cells are not human embryonic stem cells. Stem-cell-derived beta-cells display at least one marker indicative of pancreatic beta-cell (e.g. PDX-1 or NKX6.1), express insulin and display a glucose stimulates insulin secretion response (GSIS) in vitro or in vivo characteristic of an endogenous mature beta-cell.

As used herein, the term “insulin secreting beta-cell” refers to a cell differentiated from a endocrine progenitor or precursor thereof which secretes insulin. An insulin secreting beta-cells includes pancreatic beta-cells as well as pancreatic beta-like cells that express insulin.

As used herein, “pluripotent stem cell” is an undifferentiated cell which has the ability to both self-renew (through mitotic cell division) and undergo differentiation into specialized cell types deriving from the three germ layers (ectoderm, endoderm, and mesoderm) to give rise to all cells of the tissues of the body. In a particular embodiment, the pluripotent stem cell is an induced pluripotent stem cell or a human embryonic stem cell.

As used herein, the terms “iPS cell” and “induced pluripotent stem cell” are used interchangeably and refer to a pluripotent stem cell artificially derived (e.g., induced or by complete reversal) from a non-pluripotent cell, typically an adult somatic cell, for example, by inducing a forced expression of one or more genes.

By “human embryonic stem cells” or “hESC, it is herein referred to human stem cells derived from the inner cell mass (ICM) of a human embryo at the blastocyst stage. Human embryos reach the blastocyst stage 4-5 days post fertilization, at which time they consist of between 50 and 150 cells. Embryonic stem cells are pluripotent stem cells. According to the invention, human embryonic stem cells may be either obtained from an established cell line, or isolated from an embryo by different techniques known from the person skilled in the art. In some embodiments, a human embryo was not destroyed for the source of stem cell used on the methods and compositions as disclosed herein.

Methods for generating stem cells derived beta-cells are well-known in the art and are exemplified by, but not limited to, the protocols described in D'Amour, K. A. et al. (2006); Jiang, J. et al. (2007); and Kroon, E. et al. (2008), Rezania et al (2012, 2014), Felicia W. Pagliuca et al (2014) and PCT international application No. PCT/US2014/041992, the relevant part being incorporated within the present disclosure. These protocols for directing the differentiation of pluripotent stem cells into insulin-secreting beta-cells include differentiating stem cells into progenitor cells such as pancreatic progenitor or endocrine progenitor cell that can be directed to differentiate into insulin secreting beta-cells.

A cell therapy product refers to the cell composition which is administered to said patient for therapeutic purposes. Said cell therapy product include a therapeutically efficient dose of cells and optionally, additional excipients, adjuvants or other pharmaceutically acceptable carriers.

Suitable anti-diabetic or hypoglycemiant agents may include without limitation, angiotensin-converting enzyme inhibitors, angiotensin II receptor blockers, cholesterol lowering drugs, biguanides, metformine, thiazolidinediones, hypoglycemiant sulfamides, DPP-4 inhibitors, alpha-glucosidases inhibitors, insulin or their derivatives, including short-acting, rapid-acting or long-acting insulin, GLP1 analogues, derivatives of carbamoylmethylbenzoic acid; typically, insulin receptors, SLGT2 inhibtiors, GABR targeting molecules, and IL2R targeting molecule.

In accordance with the foregoing the present disclosure provides in a yet further aspect: A method as defined above comprising co-administration, e.g. concomitantly or in sequence, of a therapeutically effective amount of a Variant or related fusion protein of the Disclosure, or a functional equivalent thereof, and at least one second drug substance, said second drug substance being cytokines, anti-viral, anti-inflammatory agents, anti-diabetic agents, cell therapy product (e.g beta-cell composition), e.g. as indicated above.

In a particular embodiment of a method for treating one of the disorders disclosed above, in a subject in need thereof, said method comprising administering to said subject a therapeutically efficient amount of a Rspo1 protein or a Variant or related fusion protein of the Disclosure or a functional equivalent as disclosed above, in combination with (e.g., before, simultaneously or following) a therapeutically effective amount of a beta-cell composition, in particular stem cell-derived beta-cell composition as disclosed herein.

In a particular embodiment, said beta-cell is isolated from a live donor or a cadaveric donor.

In another particular embodiment, said beta-cell is a stem cell-derived beta-cell. Stem cell-derived beta-cell refers to insulin secreting beta-cell obtained by the differentiation of stem cells, in particular induced pluripotent stem cell, human embryonic stem cells or mesenchymal progenitor cells. In an embodiment, when stems cells are human stem cells, said human stem cells are not human embryonic stem cells.

Methods for transplanting beta-cells or islets of Langerhans to patients are for example disclosed in Shapiro, et al (2000) The New England Journal of Medicine. 343 (4): 230-238, and Shapiro et al (2017) Nature Reviews Vol 13: 268-277.

In some embodiments, beta-cells described herein are administered to said patient as dispersed cells or formed into cluster. The beta-cells may be implanted into an appropriate site in a subject, such as non-limiting examples liver, natural pancreas, renal subcapsular space, omentum, peritoneum, subserosal space, intestine, stomach or a subcutaneous pocket.

In specific embodiments, said beta-cell composition is autologous to the subject in need of the treatment, preferably derived from iPS obtained from somatic cells of the subject in need of the treatment.

In specific embodiments, said subject in need of such treatment is a subject suffering from diabetes, preferably diabetes type 1.

In another embodiment, Variant or related fusion protein of the Disclosure, or a functional equivalent thereof, can be used in in vitro methods to induce the proliferation of pancreatic beta-cells and/or islets of Langerhans.

Accordingly, in one aspect, the disclosure further provides methods for in vitro producing beta-cells said method comprising:
(i) providing beta-cells,
(ii) culturing said beta-cells in the presence of an efficient amount of said Variant or related fusion protein, or a functional equivalent thereof, under conditions to induce the proliferation of said beta-cells.

In specific embodiments of said production method, said beta-cells are primary cells, preferably from a subject in need of beta-cells therapy or transplantation of islets of Langerhans.

In other specific embodiments, said beta-cells provided at step (i) have been obtained from iPS cells, after differentiating said iPS cells into beta-cells.

Accordingly, in a particular embodiment, the disclosure relates to in vitro method for the production of beta-cells from induced pluripotent stem cells, comprises the following:
(i) providing induced pluripotent stem cells (iPSCc),
(ii) in vitro differentiating said iPSCs to p-cells of islets of Langerhans, and
(iii) culturing said differentiated beta-cells under proliferating conditions,
   wherein a sufficient amount of said Variant or related fusion protein, or a functional equivalent thereof, is added at step (ii) and/or step (iii) for differentiating iPS cells and/or inducing the proliferation of said P-cells.

Methods for differentiating iPSCs to P-cells of islets of Langerhans are already described in the art, for example in Pagliuca, et al. Cell 159, 428-439 (2014) and Rezania et al. Nat Biotechnol. 2014 November; 32(11):1121-33), the relevant part being incorporated within the present disclosure.

Said disclosure further includes the composition comprising said P-cells obtainable or as obtained by the above methods and their use as a cell therapy product, for example in a subject for treating diabete, preferably diabete type 1. Methods for transplanting beta-cells or islets of Langerhans to patients are for example disclosed in Shapiro, et al (2000) The New England Journal of Medicine. 343 (4): 230-238, and Shapiro et al (2017) Nature Reviews Vol 13: 268-277.

Also within the scope of the present disclosure are kits consisting of the compositions (e.g., comprising a Variant of the Disclosure) disclosed herein and instructions for use. The kit can further contain a least one additional reagent, or one or more additional antibodies or proteins. Kits typically include a label indicating the intended use of the contents of the kit.

The term label includes any writing, or recorded material supplied on or with the kit, or which otherwise accompanies the kit. The kit may further comprise tools for diagnosing whether a patient belongs to a group that will respond to an Rspo treatment, as defined above.

Another therapeutic strategy is based on the use of the Variant or related fusion protein, or a functional equivalent thereof, herein as agents which expand beta-cellbeta-cells isolated from a sample of a human subject.

The disclosure thus relates to a method for treating a subject in need thereof, comprising:
(a) isolating cells from a subject,
(b) optionally expanding and/or reprogramming said cells to induced pluripotent stem cells,
(c) differentiating said iPS cells to beta-cellbeta-cells, and
(d) expanding in vitro said beta-cellbeta-cells in the presence of a Variant or related fusion protein of the Disclosure, or a functional equivalent thereof, for example a recombinant protein comprising any one of SEQ ID NO:22-28, SEQ ID NO:42-43, SEQ ID NO:50-89, SEQ ID NO: 92-98, SEQ ID NO: 100-101 and SEQ ID NO: 105-111 or a functional equivalent thereof, and, optionally, other cells,
(e) optionally, collecting the expanded beta-cell, and/or formulating the expanded beta-cells and administering a therapeutically efficient amount of said expanded beta-cells to the subject.

The disclosure further relates to the use of said Variant or related fusion protein of the Disclosure, or a functional equivalent thereof, (such as a recombinant protein comprising any one of SEQ ID NO:22-28, SEQ ID NO:42-43, SEQ ID NO:50-89, SEQ ID NO: 92-98, SEQ ID NO: 100-101 and SEQ ID NO: 105-111 or a functional equivalent thereof) as agents which in vitro expand beta-cells.

The disclosure also relates to the Variant or related fusion protein of the Disclosure, or a functional equivalent thereof (such as a recombinant protein comprising any one of SEQ ID NO:22-28, SEQ ID NO:42-43, SEQ ID NO:50-89, SEQ ID NO: 92-98, SEQ ID NO: 100-101 and SEQ ID NO: 105-111 or a functional equivalent thereof), for use in vivo as an agent for inducing the proliferation of beta-cells in human, in particular in a subject that has a loss of functional beta-cells, typically a subject suffering from diabetes.

The disclosure thus relates to a method of treatment of a subject suffering from diabetes, e.g. diabetes type-1 or another disorder with a loss of functional beta-cells, said method comprising:
(i) administering in said subject an efficient amount of a Variant or related fusion protein of the Disclosure, or a functional equivalent thereof, typically a recombinant protein comprising any one of SEQ ID NO:22-28, SEQ ID NO:42-43, SEQ ID NO: 92-98, SEQ ID NO: 100-101, SEQ ID NO: 105-111,and SEQ ID NO:50-89, or a functional equivalent thereof, and,
(ii) administering an efficient amount of a P cell composition in said subject, wherein said efficient amount of said Variant or related fusion protein, has the capacity to increase the proliferation of said P cell composition. Steps (i) and (ii) can be carried out simultaneously or sequentially, in particular, either step (i) or step (ii) is first administered to said subject.

The invention having been fully described is now further illustrated by the following examples, which are illustrative only and are not meant to be further limiting.

### EMBODIMENTS

E1. A recombinant variant of R-spondin protein comprising the following FU1, FU2, TSP and BR domains, wherein
a. FU1 is a domain having at least 80% identity to any of the FU1 domains of human Rspo1, Rspo2, Rspo3 or Rspo4 as shown in SEQ ID NO: 5, 9, 13 and 17 respectively,
b. FU2 is a domain having at least 80% identity to any of the FU2 domains of human Rspo1, Rspo2, Rspo3 or Rspo4 as shown in SEQ ID NO: 6, 10, 14, 18 respectively,
c. TSP is a domain having at least 80% identity to any of the TSP domains of human Rspo1, Rspo2, Rspo3 or Rspo4 as shown in SEQ ID NO: 7, 11, 15, 19, and,
d. BR is a domain having at least 80% identify to any of BR domains of human Rspo1, Rspo2, Rspo3, or Rspo4 as shown in SEQ ID NO: 8, 12, 16, 20.

E2. The recombinant variant of E1, wherein each domain FU1, FU2, TSP and BR has at least 80% identity to the respective FU1, FU2, TSP and BR domains in human Rspo1 domain of SEQ ID NO:1.

E3. The recombinant variant of E1 or E2, wherein the TSP and BR domains have 100% identity to corresponding human Rspo1 TSP and BR domains respectively, and the FU1 and FU2 domain amino acid sequences are at least 80% identical to human Rspo1 FU1 and FU2 corresponding amino acid sequences, the difference being due one to amino acid substitutions.

E4. The recombinant variant of any one of E1 to E3, which has no more than 1, 2, 3, 4, 5, 6, 7, 8, 9, or 10 amino acid substitutions in each of the FU1 or FU2 domain when aligned with corresponding human Rspo1 FU1domain of SEQ ID NO:5 and Rspo1 FU2 domain of SEQ ID NO:6 respectively.

E5. The recombinant variant of any one of Claims E1-E4, which comprise a deletion of the first 10-14 N-terminal amino acids within the region 21-33 of Rspo1, or within an equivalent region in Rspo2, Rspo3, or Rspo4, typically a deletion of residues 21-31 of Rspo1.

E6. The recombinant variant of E5, comprising or consisting of a protein of SEQ ID NO:24.

E7. The recombinant variant of any one of E1-E6, which comprises at least an amino acid substitution of R66 in human Rspo1 FU1 domain of SEQ ID NO:5, or of the equivalent arginine residue in human Rspo2, Rspo3, or Rspo4 FU1 domain sequence, said amino acid substitution decreasing or abolishing ZNRF3 binding.

E8. The recombinant variant of Claim E7, which comprises Rspo1 FU1 domain of SEQ ID NO:5 having a single amino acid substitution of residue R66 decreasing or abolishing ZNRF3 binding, typically R66A.

E9. The recombinant variant of E7 or E8, wherein said FU2, TSP and BR domains are 95%, preferably 100% identical to Rspo1, for example said recombinant variant comprises or essentially consists of SEQ ID NO:23.

E10. The recombinant variant of any one of Claims E1-E9, wherein said variant of R-spondin binds to LGR4 with a higher affinity than human Rspo1 of SEQ ID NO:41, as measured in Rspo1/LGR4 binding affinity in vitro assay.

E11. The recombinant variant of E10, which includes one or more amino acid substitutions at position H108, N109, E116, L118, P127, A128, S133, A136, G138, or S143 of Rspo1 or corresponding residues in Rspo2, Rspo3 or Rspo4, to increase binding affinity to LGR4 as compared to a chimeric Rspo1 of SEQ ID NO:41, more specifically one or more of the following amino acid substitutions: H108K, N109D, E116V, -118F, P127D, A128F, S133F, A136L, G138E, or S143V.

E12. The recombinant variant of E10, which includes one or more amino acid substitutions at position E45, L46, E49, V50, N51, K55, S57, 162, L63, D68, P77, F84, D85, N88, or 195 of Rspo1 or corresponding residues in Rspo2, Rspo3 or Rspo4, to increase binding affinity to ZNRF3 as compared to Rspo1 of SEQ ID NO:41.

E13. The recombinant variant of any one of E1-E12, wherein said variant does not comprise an N-glycosylation site between the FU2 and TSP domain.

E14. The recombinant variant of E13, which includes a mutation in residue N137 of Rspo1 or in an equivalent residue in Rspo2, Rspo3, or Rspo4, to suppress N-glycosylation at this position.

E15. The recombinant variant of E14 which comprises or essentially consists of SEQ ID NO:22.

E16. The recombinant variant of any one of E1-E15, wherein said FU1 domain is 100% identical to Rspo2 FU1 domain of SEQ ID NO:9 and said FU2 is selected among FU2 domains of Rspo1, Rspo3, Rspo4, or their functional variants with amino acid substitutions maintaining at least the same binding affinity to LGR4.

E17. The recombinant variant of E16, comprising Rspo2 FU1 domain of SEQ ID NO:9 and Rspo1 FU2 domain of SEQ ID NO:6, and optionally Rspo1 TSP domain of SEQ ID NO:7 and Rspo1 BR domain of SEQ ID NO:8.

E18. The recombinant variant of E16, which comprises or essentially consists of SEQ ID NO:25 or SEQ ID NO:26.

E19. The recombinant variant of any one of E1-E15, having the same sequence as SEQ ID NO:9 except that it includes one or more amino acid substitutions at position E49, V50, D68, and D85 to increase binding affinity to ZNRF3 as compared to a protein of SEQ ID NO:25.

E20. The recombinant variant of E19, which comprises Rspo1 FU1 domain of SEQ ID NO:5 except that it includes one or more amino acid substitution at position E49, V50, D68, and D85, to increase binding affinity to ZNRF3 as compared to a protein of SEQ ID NO:25, preferably it includes the following amino acid substitutions E49K, V50D, D68G, and D85G, and further wherein said FU2 domain is selected among FU2 domains of Rspo1, Rspo3, Rspo4, or their functional variants with amino acid substitutions maintaining at least the same binding affinity to ZNRF3.

E21. The recombinant variant of E19 or E20, wherein said FU2 domain is 100% identical to Rspo1 FU2 domain of SEQ ID NO:6.

E22. The recombinant variant of E19, or E20, which comprises the Rspo1 FU1 and Rspo1 FU2 domains with one or more amino acid substitutions selected among E45L, E49K, V50D, K55R, D68G, D85G, N88A, and H108K, N109D.

E23. The recombinant variant of any one of E19-E22, which comprises or essentially consists of SEQ ID NO:27.

E24. The recombinant variant of any one of E19-E22, which comprises or essentially consists of SEQ ID NO:28.

E25. The recombinant variant of any one of E19-E22, which comprise human Rspo3 or Rspo4 corresponding FU2 domain of SEQ ID NO. 10, 14 or 18, in combination with Rspo2 FU1 domain or a hybrid Rspo1 FU1 domain for example including one or more amino acid substitutions at position E49K, V50D, D68G, and D85G to increase binding to ZNRF3, such as E49K, V50D, D68G, D85G.

E26. The recombinant variant of E1, which is a variant of any one of SEQ ID NO: 22-28 with no more than 10 amino acid substitutions as compared to any one of SEQ ID NO: 22-28.

E27. The recombinant variant of E26, having at least 95% identity to at least one of SEQ ID NO: 22-28, and wherein said FU1 and FU2 domains are 100% identical to the FU1 and FU2 domains of at least one of SEQ ID NO: 22-28.

E28. The recombinant variant of E26, which includes an amino acid sequence identical to one of SEQ ID NO:22-28, except for 1, 2 or 3 amino acid residues which have been replaced by another natural amino acid, preferably by conservative amino acid substitutions.

E29. The recombinant variant of any one of E1-E28, wherein said FU2 domain comprises one or more of the following amino acid residues: F106, H108, F110, N109, E116, L118, P127, A128, S133, A136, G138, S143 in human Rspo1 of SEQ ID NO:1 or the corresponding residues in human Rspo2 of SEQ ID NO:2, human Rspo3 of SEQ ID NO:3 or human Rspo4 of SEQ ID NO:4.

E30. The recombinant variant of any one of E1-E29, wherein said BR domain comprises at least one or more amino of the following amino acid residues T253, L257, T258, S259, A260 or A263, typically said BR domain is 100% identical to SEQ ID NO: 8, 12, 16, 20.

E31. The recombinant variant of any one of E1-E30, which includes amino acid substitutions at the BR domain to improve O-glycosylation, for example, wherein said variant comprises one or more amino acid substitutions at position G252, T253, L257, T258, S259, A260, A263 of Rspo1 or corresponding residues in Rspo2, Rspo3 or Rspo4, for example, said variant includes a BR domain of Rspo1 with one or more of the following amino acid substitutions G252T, L257S, A260T or A263T.

E32. The recombinant variant of any one of E1-E31, which exhibit one or more of the following properties to a level at least similar to Rspo1 protein of SEQ ID NO: 41: (i) it binds to LGR4 receptor with at least the same affinity as reference human Rspo1 of SEQ ID NO:41, as measured in Rspo1/LGR4 binding affinity in vitro assay, for example as determined by SPR or ELISA assay;
(ii) it binds to ZNRF3 receptor with at least the same affinity as reference human Rspo1 of SEQ ID NO:41, as measure in Rspo1/ZNRF3 binding affinity in vitro assay, for example as determined by SPR or ELISA assay;
(iii) it activates wnt/beta catenin pathway to at least a similar level as reference human Rspo1 of SEQ ID NO:41, for example as determined in a Top Flash assay as described in the examples below,
(iv) it induces the proliferation of functional beta-cells to at least a similar level as reference human Rspo1 of SEQ ID NO:41, for example as determined in an in vitro Min6 beta-cell proliferation assay; and/or,
(v) it induces the proliferation of functional beta-cells to at least a similar level as reference human Rspo1 of SEQ ID NO:41, for example as determined in an in vivo beta-cell proliferation assay.

E33. An Fc fusion protein which includes a recombinant variant of any one of E1-E32, with an Fc fragment fused directly, or indirectly via a peptide linker, to said recombinant variant.

E34. The Fc fusion protein of E33, which further includes an Fc fragment fused directly or indirectly at the N-terminal end of the R-spondin protein.

E35. The Fc fusion protein of E33, which further includes an Fc fragment fused directly or indirectly at the C-terminal end of the R-spondin protein.

E36. The Fc fusion protein of any one of E33-35, wherein said peptide linker include for example the linker of (GGGGGGSGGGGSGGGGSA) (SEQ ID NO:44), (GGGGSGGGGSGGGGGG) (SEQ ID NO:45), GGGGGGSGGGGSA (SEQ ID NO: 102), GGGSGGGGSA (SEQ ID NO: 103), SGGGGSA (SEQ ID NO: 104), GGGGS (SEQ ID NO: 46), GG or SA.

E37. The Fc fusion protein of any one of E33-E36, wherein said Fc fragment comprises or essentially consists of SEQ ID NO:29.

E38. The Fc fusion protein of any one of E33-E37, which comprises or essentially consists of a sequence selected from the group consisting of: SEQ ID NO:42, 43, or SEQ ID NO: 92, 93, 95-98, SEQ ID NO: 100 or 101 or SEQ ID NO: 105 to 111.

E39. The recombinant variant of any one of E1-E38, for use as a drug, in particular for treating diabetes, more preferably diabetes type I or II in a patient in need thereof.

E40. The fusion protein of any one of E33-E39, for use as a drug, in particular for treating diabetes, more preferably diabetes type I or II in a patient in need thereof.

E41. The recombinant variant for use of claim E39 or the fusion protein for use of E40, wherein said recombinant variant or fusion protein is administered in said patient in combination with a beta-cell, preferably a stem cell-derived beta-cell.

E42. A nucleic acid encoding a recombinant variant of any one of E1-E32.

E43. A vector comprising a nucleic acid of E42.

E44. A host cell, comprising a nucleic acid of E42.

E45. A method for producing a recombinant variant of any of E1-E32, comprising (i) culturing a host cell of E44 under conditions for expression of said recombinant variant, (ii) recovering said recombinant variant, (iii) optionally purifying said recombinant variant.

E46. A nucleic acid encoding a fusion protein of any one of E33-E38.

E47. A vector comprising a nucleic acid of E46.

E48. A host cell, comprising a nucleic acid of E46.

E49. A method for producing a fusion protein of any of E33-E38, comprising (i) culturing a host cell of E44 under conditions for expression of said fusion protein, (ii) recovering said recombinant variant, (iii) optionally purifying said recombinant variant.

### DESCRIPTION OF THE FIGURES

FIG. 1 provides an alignment of Rspo1, Rspo2, Rspo3 and Rspo4 amino acid sequence with a schematic view of the different domains FU1, FU2, TSP and BR.

FIG. 2 Quantification of Min6 cells incubated with PBS (control in black) or recombinant hRspo1 at different doses.

FIG. 3 Quantification of Min6 cells incubated with PBS (control, black bar on the left), or different doses of variant #009 (in grey). The right bar at 400 nM corresponds to the result of Min6 quantification with wild type hRspo1.

FIG. 4 Quantification of Min6 cells incubated with PBS (control, black bar on the left), or different doses of variant #008 (in grey). The right bar at 400 nM corresponds to the result of Min6 quantification with wild type hRspo1.

FIG. 5 Quantification of Min6 cells incubated with PBS (control, black bar on the left), or different doses of variant #005 (in grey). The right bar at 400 nM corresponds to the result of Min6 quantification with wild type hRspo1.

FIG. 6 Quantification of Min6 cells incubated with PBS (control, black bar on the left), or different doses of variant #034 (in grey). The right bar at 400 nM corresponds to the result of Min6 quantification with wild type hRspo1.

FIG. 7 Quantification of Min6 cells incubated with PBS (control, black bar on the left), or different doses of variant #047 (in grey). The right bar at 400 nM corresponds to the result of Min6 quantification with wild type hRspo1.

FIG. 8 Quantification of Min6 cells incubated with PBS (control, black bar on the left), or different doses of variant #051 (in grey). The right bar at 400 nM corresponds to the result of Min6 quantification with wild type hRspo1.

FIG. 9 Quantification of Min6 cells incubated with PBS (control, black bar on the left), or different doses of variant #063 (in grey). The right bar at 400 nM corresponds to the result of Min6 quantification with wild type hRspo1.

FIG. 10 Quantification of Min6 cells incubated with PBS (control, black bar on the left), or different doses of variant #064 (in grey). The right bar at 400 nM corresponds to the result of Min6 quantification with wild type hRspo1.

FIG. 11 Quantification of Min6 cells incubated with PBS (control, black bar on the left), or different doses of variant #049 comprising a tag histidine (in grey). The right bar at 400 nM corresponds to the result of Min6 quantification with wild type hRspo1.

FIG. 12 Quantification of Min6 cells incubated with PBS (control, black bar on the left), or different doses of variant #054 (in grey). The right bar at 400 nM corresponds to the result of Min6 quantification with wild type hRspo1.

FIG. 13 Quantification of Min6 cells incubated with PBS (control) or tagless Variant #049 at 200 nM and 400 nM for 24 hours.

FIG. 14 Quantification of Min6 cells incubated with PBS (control) or tagless Variant #049 at 200 nM and 400 nM for 48 hours. Fresh protein was added after initial 24-hour incubation.

FIG. 15 Quantification of Min6 cells incubated with PBS (control) or tagless Variant #049 at 200 nM and 400 nM for 72 hours. Fresh protein was added after initial 24-hour incubation and again after 48 hours.

FIG. 16 Quantification of Min6 cells incubated with PBS (control, black bar on the left), or different doses of variant #056 (in grey). The right bar at 400 nM corresponds to the result of Min6 quantification with wild type hRspo1.

FIG. 17 Quantification of Min6 cells incubated with PBS (control, black bar on the left), or different doses of variant #082 (in grey). The right bar at 400 nM corresponds to the result of Min6 quantification with wild type hRspo1.

FIG. 18 Quantification of Min6 cells incubated with PBS (control, black bar on the left), or different doses of variant #116 (in grey). The left bar at 400 nM corresponds to the result of Min6 quantification with wild type hRspo1 (#014).

FIG. 19 Percentage of BrdU positive beta-cells in 129/Sv mouse islets of Langerhans after 4 days of culture and 72 h of treatment with Variant #014 (A), #49 (B), #008 (C), #064 (D), #051 (E), #121 (F) and GLP1 (1 nM) (G).

FIG. 20 Glycemia follow-up and diabetes incidence of female NOD mice daily treated 16 weeks with variant #014 (A, B) and variant #064 (C, D) intraperitoneal injection.

FIG. 21 Diabetes incidence in female NOD mice treated daily with variant #049 at 0.2 mg/Kg, 0.4 mg/Kg and 0.8 mg/Kg.

FIG. 22 Glycemia follow-up of NOD mice treated daily with variant #049 at 0.2 mg/Kg, 0.4 mg/Kg and 0.8 mg/Kg.

FIG. 23 Glycemia follow-up of female NOD mice weekly treated for 13 weeks with variant #121 intraperitoneal injection at a concentration of 2400 μg/Kg.

FIG. 24 Diabetes incidence in female NOD mice weekly treated for 13 weeks with variant #121 intraperitoneal injection at a concentration of 2400 μg/Kg.

FIG. 25 Beta cell mass quantification in NOD mice treated for 13 weeks daily with protein #049 at 200 μg/Kg, 400 μg/Kg and 800 μg/Kg and weekly with protein #121 at 2400 μg/Kg.

FIG. 26 Quantification of Ki67/insulin double positive cells normalized for islet area (μm²) on pancreatic sections from mice injected intraperitoneally with variant #014 for 5 consecutive days.

FIG. 27 Quantification of BrdU/insulin double positive cells normalized for islet area (μm²) on pancreatic sections from mice injected intraperitoneally with variant #014 for 5 consecutive days.

FIG. 28 Glycemia follow-up of STZ-induced hyperglycemic mice daily treated with variant #014 intraperitoneal injection, starting 15 days before STZ treatment.

FIG. 29 Water-intake of STZ-induced hyperglycemic mice daily treated with variant #014 intraperitoneal injection, starting 15 days before STZ treatment.

FIG. 30 Intraperitoneal glucose tolerance test performed on STZ-induced hyperglycemic mice daily treated with variant #014 intraperitoneal injection, starting 15 days before STZ treatment. This test was carried out 74 days after the beginning of variant #014 daily administration.

FIG. 31 Glycemia follow-up of STZ-induced hyperglycemic mice daily treated with variant #014 intraperitoneal injection, starting 7 days after STZ treatment.

FIG. 32 Water-intake of STZ-induced hyperglycemic mice daily treated with variant #014 intraperitoneal injection, starting 7 days after STZ treatment.

FIG. 33 Intraperitoneal glucose tolerance test performed on STZ-induced hyperglycemic mice daily treated with variant #014 intraperitoneal injection, starting 7 days after STZ treatment. This test was carried out 63 days after the beginning of variant #014 daily administration.

FIG. 34 Fasting human c-peptide measured in blood of animals transplanted with human islets and daily injected with either PBS or variant #014 at 0.4 mg/Kg and 0.8 mg/Kg.

FIG. 35 Intraperitoneal glucose tolerance test performed on mice transplanted with human islets after 28 days of treatment with PBS (controls) or variant #014 at 0.4 mg/Kg and 0.8 mg/Kg

FIG. 36 Basal and glucose-stimulated blood human c-peptide levels of mice transplanted with human islets after 28 days of treatment with PBS (controls) or variant #014 at 0.4 mg/Kg and 0.8 mg/Kg

FIG. 37 Quantification of total insulin volume in transplanted human islets after 60 days of treatment with PBS (controls) or variant #014 at 0.4 mg/Kg and 0.8 mg/Kg

FIG. 38 Comparison of 168 hour-kinetic of #63-1, #63-2 and #64 Fc-fusion proteins after SC administration at 0.8 mg/kg in male 129/Sv mice (n=3/timepoint) showing extension of half-life in relation to reference non-Fc protein #14. Single-dose PK profile protein #14 is presented (dashed line) for comparison.

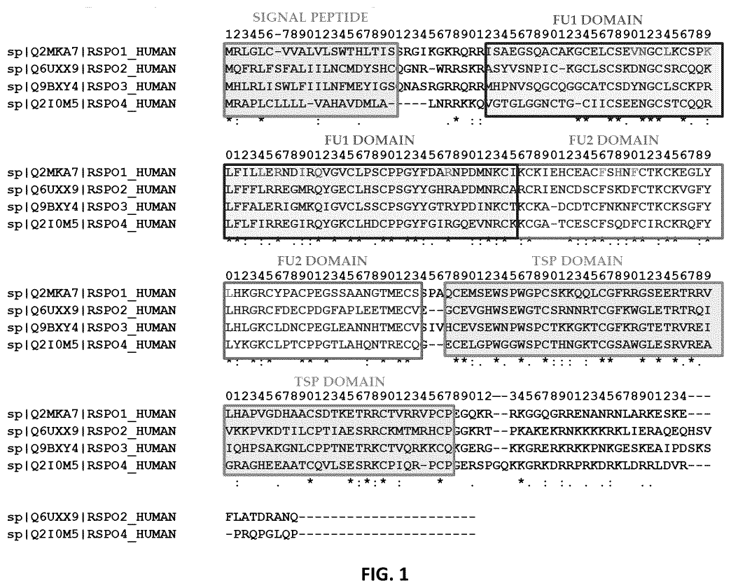

### EXAMPLES

1. Functional Tests for Characterizing R-Spondin Proteins

ZNRF3 Binding Assay: ELISA (Enzyme-Linked Immunosorbent Assay) Protocol

ELISA is a plate-based assay for detecting and quantifying soluble proteins (ligands) in a liquid sample using ligand binding molecules (antibodies, fusion-molecules etc) and detection antibodies.

Procedure:

ELISA modules (Nunc Maxisorp, Thermo Scientific) are processed with antigens (100 μl/well, 1 μg/ml in PBS pH 7.4, overnight at 4° C.). After capturing the antigens, the plate is washed 4 times with PBS pH 7.4, containing 0.05% Tween 20 (PBS-Tween) and blocked with PBS-Tween, containing 2% BSA (Merck) for 60 min. Then, a serial two-fold dilution of the samples (see Used molecules for the second layer) is performed in PBS-Tween containing 1% BSA starting from 4000 ng/ml, applied to the plate (100 μl/well) and incubated for 60 min on a shaker (300 rpm) at room temperature.

If third layer is necessary (LGR4-His binding): The plates are washed 4 times with PBS-Tween, followed by the addition of 100 μl of antibody (see Used antibody for the third layer), diluted in PBS-Tween containing 1% BSA. The plate is incubated for 30 min on a shaker (300 rpm) at room temperature.

Next, the plates are washed 4 times with PBS-Tween, followed by the addition of 100 μl of peroxidase conjugated goat anti-mouse IgG polyclonal antibody (Goat anti-mouse-IgG-HRP, 1:10 000, 0.1 μg/mL, LabAs Estonia), diluted in PBS-Tween containing 1% BSA. The plate is incubated for 30 min on a shaker (300 rpm) at room temperature.

The plate is washed 4 times with PBS-Tween, followed by addition of 100 μl of TMB substrate solution VII (Biopanda Diagnostics). The reaction is allowed to develop for 10 min on a shaker (300 rpm) at RT and terminated by adding 50 μl of 0.5 M sulfuric acid.

The absorbance is measured at 450 nm by ELISA plate reader (Thermo Scientific).

Used antigens:
RSPO-1 (#120-38, Peprotech)
RSPO-2 (#3266-RS, R&D Biosystems)
RSPO-3 (#120-44, Peprotech)
Proteins #007, #008, #014, #059 produced at Icosagen Cell Factory
Used molecules for the second layer
LGR4-His (#LG4-H52H3, Acro)
LGR4-Fc (#7750-GP, R&D Biosystems)
ZNRF3-Fc (#7994-RF, R&D Biosystems)
Used antibody for the third layer (for LGR4-His binding):
Mouse monoclonal anti-His antibody (1:2500, 0.2 μg/mL, GenScript, Cat. No. A00186)

TOP-FLASH-ASSAY for Measuring Wnt/β-Catenin Signaling Pathway Activity

The Super-Top-Flash luciferase reporter assay can be used to monitor the concentration and activity of both Wnt and R-spondin proteins in conditioned media. At first Super-TOP-Flash reporter-expressing HEK 293 STF cells are prepared and plated in serum-free DMEM onto 96-well plates (1-2×10⁴ cells per well). After 24h, serum-starved reporter cells are exposed to different amounts of culture medium containing either Wnt or R-spondin proteins. After 18-24 hours of induction, luciferase activity is measured and the amount of growth factors present in the conditioned media is compared to a known source of protein (recombinant standard hRSPO1). This cell-based reporter assay can test the activity of both Wnt and R-spondin ligands.

Required Reagents and Cell Line:
a. HEK-293 STF cells (ATCC).
b. R-spondin proteins produced at Icosagen Cell Factory and RSPO-1 (#120-38, Peprotech), RSPO-2 (#3266-RS, R&D Biosystems), RSPO-3 (#120-44, Peprotech)
d. Steady-Glo® Luciferase Assay System (Promega, cat #E2510)
e. complete growth media: DMEM (Gibco) cell culture media, containing 10% FCS (Gibco)+1× Pen/Strep
f. serum-free growth media: DMEM (Gibco) culture media+1× Pen/Strep
g. CELLSTAR® 96 well plates: flat bottom white polystyrene wells (Greiner) Preparation of HEK-293 STF cells for Super-Top-Flash luciferase reporter assay:

Day 0: Detach HEK-293-STF cells with 2 ml trypsin solution from the bottom of the 10 cm culture plate, after 5 minutes add 2 ml of complete growth media. Spin down the cells at 200 g for 5 min and suspend the cell pellet in serum-free DMEM. Count the cells and seed HEK-293-STF reporter cells (2*10⁵ cells/ml) in a total volume of 100 ul serum-free media into CELLSTAR 96-well plates. Cultivate the cells for 22-26h at 37 C in a humidified cell-culture incubator.

Day 1: Dilute the R-spondin proteins into DMEM culture media and apply to the pre-cultured HEK-293 STF cells so that a serial dilution is achieved (in this case three-fold dilutions were used). Cultivate the cells with the inducer for 18-24h at 37 C.

Day 2: Measure STF firefly activity by adding 50 μl of Steady-Glo® Luciferase Assay substrate directly to culture wells, wait for at least 5 min, measure bioluminescence signal intensity using Glomax Explorer luminometer (Promega). Export the results to PC for analysis.

Min6 Cell Proliferation Assay

Mouse insulinoma (Min6) cells are cultured in DMEM supplemented with 4.5 g/l of glucose, 15% of FCS (fetal calf serum), 0.005% of sodium bicarbonate, 100 U/mL penicillin and 100 mg/mL streptomycin and maintained in a humidified atmosphere (37° C.; 95% air/5% CO2). Low-passage (max P30) Min6 cells are plated into a 12-well plate at a seeding density of 80000 cells/ml. Adherent cells are incubated with target molecules diluted at different concentrations into low-FCS medium (7.5%) for 24 hours. For longer incubations, fresh protein was added to culture medium every 24 hours. Cells are finally detached and manually quantified using a Thoma Chamber.

A variant is considered more efficient than native hRspo1 when it either displays an overall stronger proliferative induction capability as compared to Rspo1 at 400 nM or when the concentration allowing significant proliferation increase is lower when compared to a similar concentration of hRspo1.

Wnt/Beta Catenin Assay

Min6 cells are cultured as described in the previous paragraph. Low passage cells are plated into a 6-well plate at a seeding density of 250000 cells/ml. Adherent cells are treated with different doses of hRspo1 analogs into low-FCS medium (5%) at several time points. Subsequently, cells are detached and resuspended into PBS. Total protein content is isolated by sonication and the concentration of β-catenin is assessed by ELISA assay, following manufacturer's instructions. BCA content is used to normalize each protein sample.

In Vivo Proliferation Assay

In vivo proliferation tests are performed on 2 months-old wild-type 129SV mice. Following a minimum acclimatation period of 3 days, animals are daily injected intraperitoneally or subcutaneously with the variant of interest at different concentration for several consecutive days. Mice treated with daily injections of 150 ul of sterile PBS are used as controls. Finally, mice are sacrificed 30 minutes after last injection by cervical dislocation. Pancreata are collected and fixed in Antigenfix (paraformaldehyde solution pH 7.2-7.4; Microm Microtech France), washed in cold PBS and incubated 1 hour in 0.86% saline. Following dehydration through a series of increasing ethanol dilutions (50%, 70%, 80%, 90%, and 100%), the pancreata are treated with isopropanol and toluene and embedded in paraffin. Paraffin blocks are sectioned into 6 μm slides and analyzed by immunofluorescence using antibodies against insulin, PC1/3, Ki67 and BrdU.

A variant is considered more active than original hRspo1 if it induces a higher number of proliferative β-cells, a greater increase of the beta-cell mass, a better glucose handling, or if the onset of action/concentration required is lower than with native hRspo1.

Obviously, such approach can also be used in different diabetic mouse models, including among others, Streptozotocin-treated mice, NOD animals or Rip-B7 animals (King, Br J Pharmacol. 2012 June; 166(3): 877-894 and Karges et al., Diabetes 2002 November; 51(11): 3237-3244).

Results

A. Production of Recombinant Variants

Table 2 below list the recombinant Rspo proteins that were produced according to the protocol below.

TABLE 2
Examples of the invention and comparative examples of Rspo recombinant proteins

| | Amino acid | |
|---|---|---|
| Example | SEQ ID | Brief Description |
| #005 | SEQ ID NO: 22 | Rspo1 21-263 with N137Q (non-N-glycosylated) |
| #008 | SEQ ID NO: 23 | Rspo1 21-263 with R66A single mutation |
| #007 | — | Rspo1 21-263, Fu2 Mutant F106A |
| #009 | SEQ ID NO: 24 | Rspo1 with deletion of residues 21-31 |
| #010 | — | hRspo1 32-227 |
| #014 | SEQ ID NO: 41 | hRspo1 21-263. Reference protein |
| #018 | — | Rspo1 32-263 with N137Q (non-N-glycosylated) |
| #020 | SEQ ID NO: 3 | Full length human Rspo3 (3-33-272-His-NSP) |
| #021 | — | Full length human Rspo3 (3-33-272) |
| #026 | SEQ ID NO: 50 | Chimeric: RSPO1Fu1_32-95-RSPO3Fu2_97-143-RSPO1_144- |
| | | 263 |
| #027 | SEQ ID NO: 51 | Chimeric: RSPO1Fu1_32-90-RSPO3Fu2_92-143-RSPO1_144- |
| | | 263 |
| #028 | SEQ ID NO: 52 | Chimeric: RSPO3Fu1_33-96-RSPO1Fu2_96-143-RSPO1_144- |
| | | 263 |
| #029 | SEQ ID NO: 53 | Chimeric: RSPO3Fu1_33-91-RSPO1Fu2_91-143-RSPO1_144- |
| | | 263 |
| #030 | SEQ ID NO: 54 | Chimeric. RSPO3Fu1_33-96-RSPO1Fu2_96-143-RSPO1_144- |
| | | 263 N137Q mutation. No N-Glycosylation |
| #031 | SEQ ID NO: 55 | Chimeric: RSPO2Fu1-Fu2_32-142-RSPO1_144-263 |
| #032 | SEQ ID NO: 56 | Chimeric Fu2-Rspo2 into Rspo1 |
| #033 | SEQ ID NO: 57 | Chimeric: RSPO1Fu1_32-90-RSPO2Fu2_90-142-RSPO1_144- |
| | | 263 |
| #034 | SEQ ID NO: 25 | Chimeric Rspo2 FU1 domain with Rspo1 FU2 domain |
| #035 | SEQ ID NO: 26 | Identical to #034 but with different FU1 and FU2 junction |
| #036 | SEQ ID NO: 58 | Chimeric: RSPO4Fu1-Fu2_27-136-RSPO1_144-263 |
| #037 | SEQ ID NO: 59 | Chimeric Fu2-Rspo4 into Rspo1 |
| #038 | SEQ ID NO: 60 | Chimeric: RSPO1Fu1_32-90-RSPO4Fu2_85-136-RSPO1_144- |
| | | 263 |
| #039 | SEQ ID NO: 61 | Chimeric: RSPO4Fu1_27-89-RSPO1Fu2_96-143-RSPO1_144- |
| | | 263 |
| #040 | SEQ ID NO: 62 | Chimeric: RSPO4Fu1_27-84-RSPO1Fu2_91-143-RSPO1_144- |
| | | 263 |
| #042 | SEQ ID NO: 63 | Chimeric: RSPO2Fu1_32-76-RSPO4Fu2_72-136-RSPO1_144- |
| | | 263 |
| #046 | SEQ ID NO: 64 | hRSPO1 21-263-short-Linker-Peptide with high affinity for |
| | | Albumin |
| #047 | SEQ ID NO: 27 | hRspo1 32-263 with 4 additional amino acid substitutions E49K, |
| | | V50D, D68G, D85G |
| #048 | SEQ ID NO: 65 | Mutant in Fu1: E45L, E49K, V50D, K55R, D68G, D85G, N88A |
| #049 | SEQ ID NO: 66 | Mutant in Fu2: H108K, N109D |
| #050 | SEQ ID NO: 67 | Mutant in Fu1 and Fu2: E49K, V50D, D68G, D85G, H108K |
| #051 | SEQ ID NO: 28 | hRspo1 32-263with 9 additional amino acid substitutions E45L, |
| | | E49K, V50D, K55R, D68G, D85G, N88A, H108K, N109D |
| #052 | SEQ ID NO: 68 | Rspo1 32-263 Mutants in Fu1: E49K, V50D |
| #053 | SEQ ID NO: 69 | hRspo1 32-263 Mutants in Fu1: D68G, D85G |
| #054 | SEQ ID NO: 70 | hRspo1 32-263 Mutant in Fu2: H108K |
| #055 | SEQ ID NO: 71 | hRspo1 32-263 Mutant in Fu1: L46S, S57Q, I62F, L63F, P77H, |
| | | F84Y, I95A |
| #056 | SEQ ID NO: 72 | hRspo1 32-263 Mutant in Fu 2: E116V, L118F, P127D, A128E |
| | | S133F A136L, G138E, S143V |
| #057 | SEQ ID NO: 73 | hRspo1 32-263 Mutant in Fu1 and Fu2: L46S, S57Q, |
| | | I62F; L63F, P77H, F84Y, I95A, E116V, L118F, P127D A128E, |
| | | S133F, A136L, G138E, S143V |
| #059 | — | Rspo1 21-263 Double mutant FU1 R66A, Q71A |
| #063 | SEQ ID NO: 42 | hRspo1 21-263-Linker- Fc |
| #064 | SEQ ID NO: 43 | Fc-Linker-hRspo1-21-263 |
| #068 | SEQ ID NO: 74 | Mutant in Fu2: H108R |
| #069 | SEQ ID NO: 75 | Mutant in Fu2: N109D |
| #071 | SEQ ID NO: 76 | Mutant in the BR Domain: L227A |
| #072 | SEQ ID NO: 77 | Mutant in the BR Domain: T253A |
| #073 | SEQ ID NO: 78 | Mutant in the BR Domain: T253A, T258A, S259A, S268A if |
| | | Linker at C-terminal is present |
| #074 | SEQ ID NO: 79 | Mutant in the BR Domain: G252T |
| #075 | SEQ ID NO: 80 | Mutant in the BR Domain: L257S |
| #076 | SEQ ID NO: 81 | Mutant in the BR Domain: A260T |
| #077 | SEQ ID NO: 82 | Mutant in the BR Domain: A263T |
| #078 | SEQ ID NO: 83 | Mutant in the BR Domain: G252T, L257S, A260T, A263T if |
| | | Linker at C-terminal is present |
| #079 | SEQ ID NO: 84 | Mutant in the BR Domain: T253E |
| #080 | SEQ ID NO: 85 | Mutant in the BR Domain: T253E, T258E, S259E, S268E |
| #081 | SEQ ID NO: 86 | Mutant in the BR Domain: N253GTN256 (Native sequence is |
| | | T253 VGP256) |
| #082 | - | Rspo1 with N-Terminal deletion 32-263 and R66A single |
| | | mutation |
| #084 | - | Protein #26 with Fu1 mutation R66A |
| #086 | — | Protein #32 with Fu1 mutation R66A |
| #088 | — | Protein #37 with Fu1 mutation R66A |
| #093 | — | hRSPO1-32-263-Q71A Mutation in Fu1 |
| #094 | SEQ ID NO: 87 | hRSPO1 21-263-Long Linker-Peptide with high affinity for |
| | | Albumin |
| #095 | SEQ ID NO: 88 | Peptide with high affinity for Albumin-Linker-hRSPO1 21-263 |
| #096 | - | hRspo1 21-263 Q71A mutation in Fu1 |
| #106 | - | Mutant #051 with additional mutation R66A |
| #116 | SEQ ID NO: 94 | #116 hRspo1 32-244 |
| #121 | SEQ ID NO: 92 | #049 with Fc at the N-terminal |
| #155 | SEQ ID NO: 93 | #049-GGGGGGSGGGGSGGGGSA-Fc |
| #194 | SEQ ID NO: 106 | #049-GGGGGGSGGGGSA-Fc |
| #193 | SEQ ID NO: 107 | #049-GGGSGGGGSA-Fc |
| #192 | SEQ ID NO: 108 | #049-SGGGSA-Fc |
| #200 | SEQ ID NO: 101 | #049-SA-Fc |
| #190 | SEQ ID NO: 109 | hRspo1 21-263-GGGGGGSGGGGSA-Fc |
| #189 | SEQ ID NO: 110 | hRspo1 21-263-GGGSGGGGSA-Fc |
| #188 | SEQ ID NO: 111 | hRspo1 21-263-SGGGGSA-Fc |
| #195 | SEQ ID NO: 96 | #009 with the FC at the N-terminal |
| #198 | SEQ ID NO: 105 | #009-GGGGGGSGGGGSA-Fc |
| #199 | SEQ ID NO: 100 | #009-SA-Fc |

All the above recombinant proteins were expressed using the QMCF technology (see also U.S. Pat. Nos. 7,790,446, 8,377,653) as developed by Icosagen, including CHO or HEK293 based QMCF cell line.

The Majority of the proteins described in Table 2 include a short linker and a C-His Tag to ease purification, at their C terminal end, after the BR domain with the exceptions of:
#014 (native hRspo1),
#46, which is functionalized at C terminal with a short linker and a sequence with high affinity for albumin,
Fc Conjugated proteins.

The skilled person would be able to produce similar recombinant proteins with alternative linker and/or tag for purification, or without such linkers and tags.

B. Determining Beta-Catenin Pathway Stimulation in a Top Flash Assay

All produced variants were tested for their capacity to stimulate beta-catenin pathway and compared with native hRspo1.

The table 3 below shows the results for some of the produced variants:

TABLE 3
Min6 proliferative activity of each tested examples.

| Protein | Topflash result at High doses | Topflash result at Low doses |
|---|---|---|
| ID | 3000; 1000; 333 ng/ml | 111.1; 37; 12.3, 4.1 ng/ml |
| #005 | ++ | ++ |
| #007 | − | − |
| #008 | + | + |
| #009 | ++ | +++ |
| #010 | ++ | ++ |
| #014 | ++ | ++ |
| #018 | ++ | ++ |
| #034 | + | +++ |
| #046 | + | ++ |
| #047 | ++ | +++ |
| #049 | ++ | +++ |
| #051 | ++/+++ | +++ |
| #054 | ++ | ++ |
| #056 | ++ | ++ |
| #059 | − | − |
| #063 | ++ | ++ |
| #064 | ++ | ++ |
| #073 | ++ | ++ |
| #116 | ++ | ++ |

(−: No detectable activity, +: Activity lower than native hRspo1 (#014), ++: Activity similar to native hRspo1 (#014), +++: Activity higher than native hRspo1 (#014))

C. Determining Min Proliferative Activity Using the In Vitro Min6 Proliferation Assay

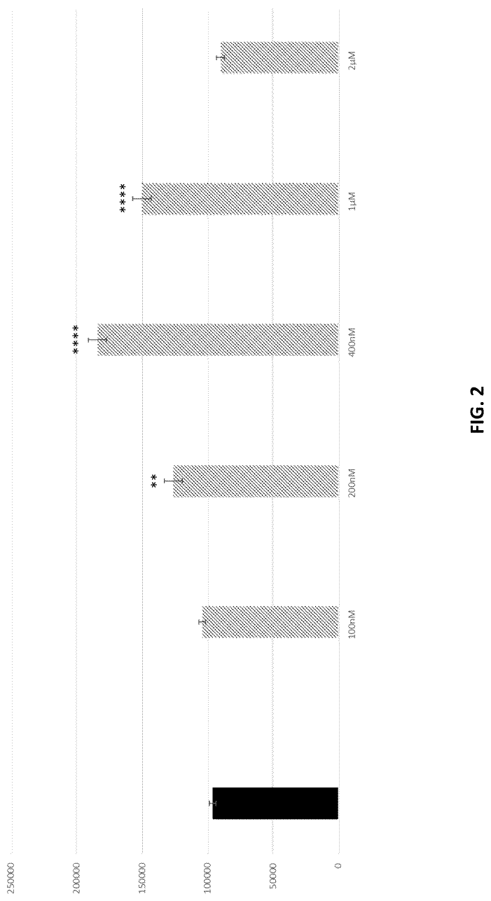

Proliferative activity of each variant was assessed in vitro using mouse insulinoma (Min6) cell line. Recombinant hRspo1 stimulates Min6 proliferation delivering a bell-shaped dose-response curve, with its peak at 400 nM (FIG. 2).

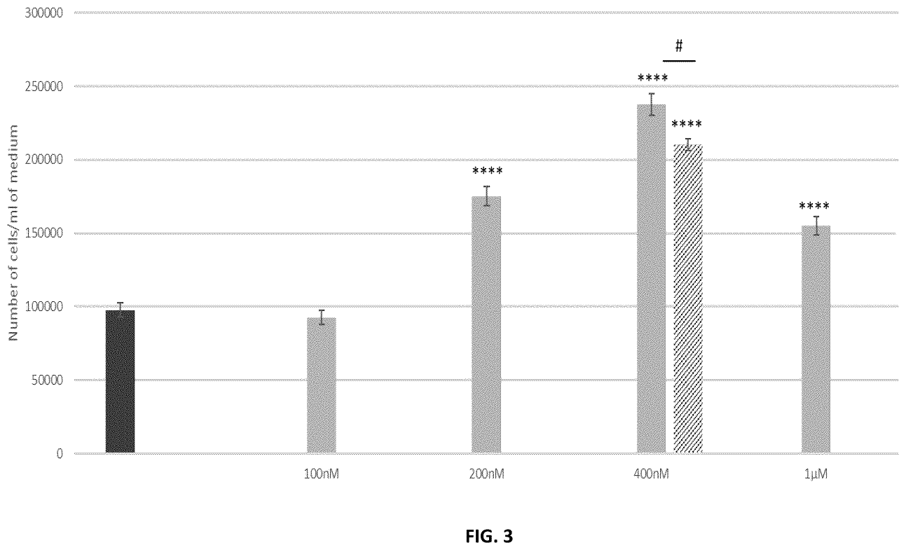

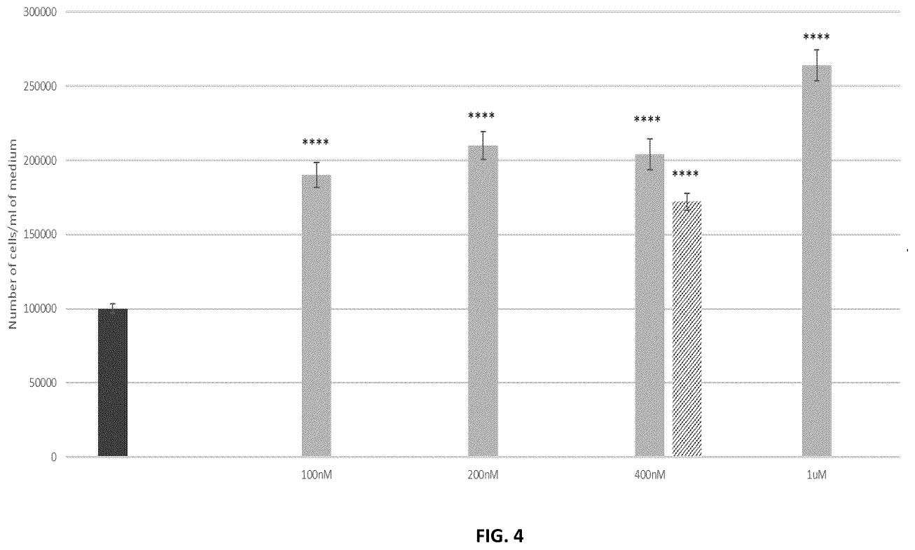

Similar to native hRspo1, variant #009 displayed a bell-shaped dose-response mitotic effect on Min6 cells, with a peak at 400 nM (FIG. 3). At this dose, however, the proliferation rate of Min6 was significantly higher compared to original hRspo1 (FIG. 3).

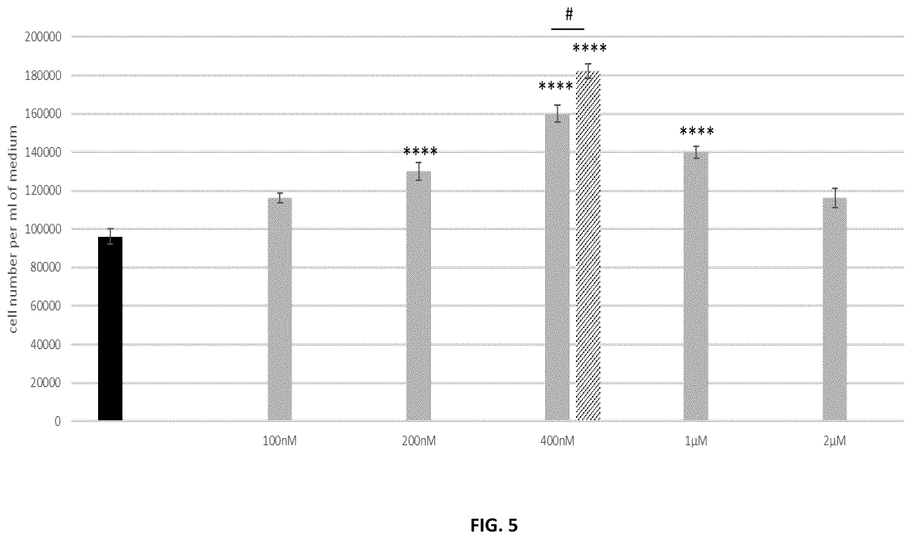

The number of Min6 cells was significantly increased upon incubation with variant #008 at all concentration tested (FIG. 4). Interestingly, this number was also significantly higher at 200 nM and 1p M doses, when compared to Min6 cells treated with hRspo1 (FIG. 4).

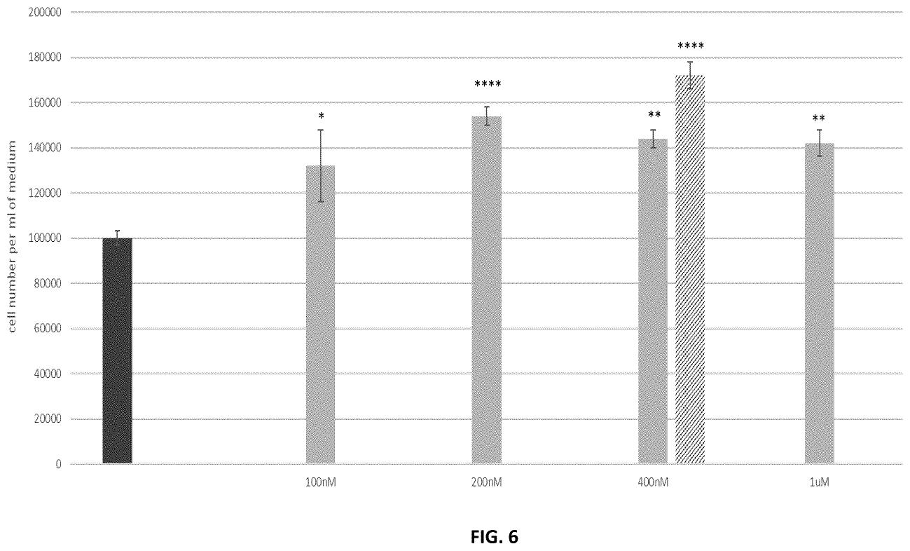

Variant #005 showed the same bell-shaped dose-response curve as the standard hRspo1, with a peak proliferative response at 400 nM (FIG. 5). Nevertheless, Min6 cells incubated with variant #005 were significantly lower in number than those incubated with the native hRspo1 (FIG. 5).

Intriguingly, variant #034 was significantly stimulating Min6 cell proliferation at all concentrations tested, with no significant differences compared to W.T. hRspo1 at 400 nM (FIG. 6).

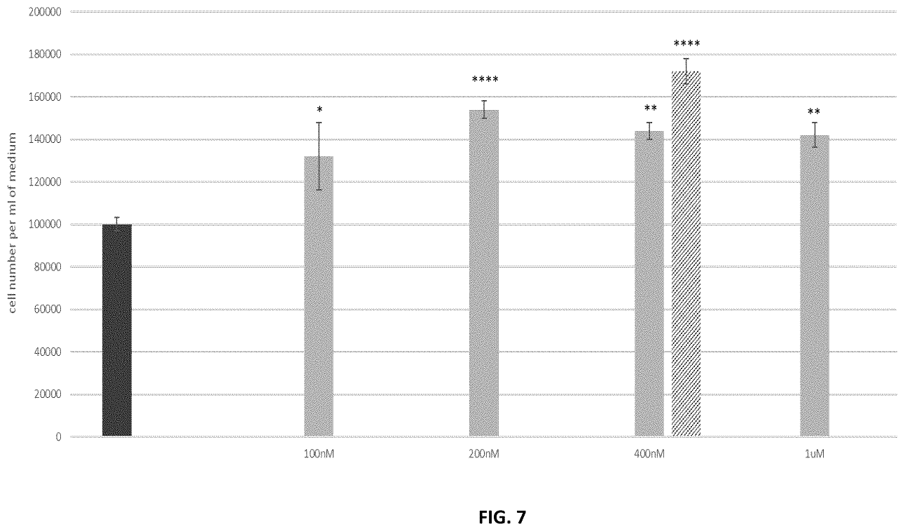

Proliferative dose-response curve of variant #047 was bell-shaped, with an onset of action at 200 nM and a peak at 1 μM (FIG. 7). At this latter concentration, the mitotic rate of Min6 cells was found significantly augmented compared to cells incubated with original hRspo1 at 400 nM (FIG. 7).

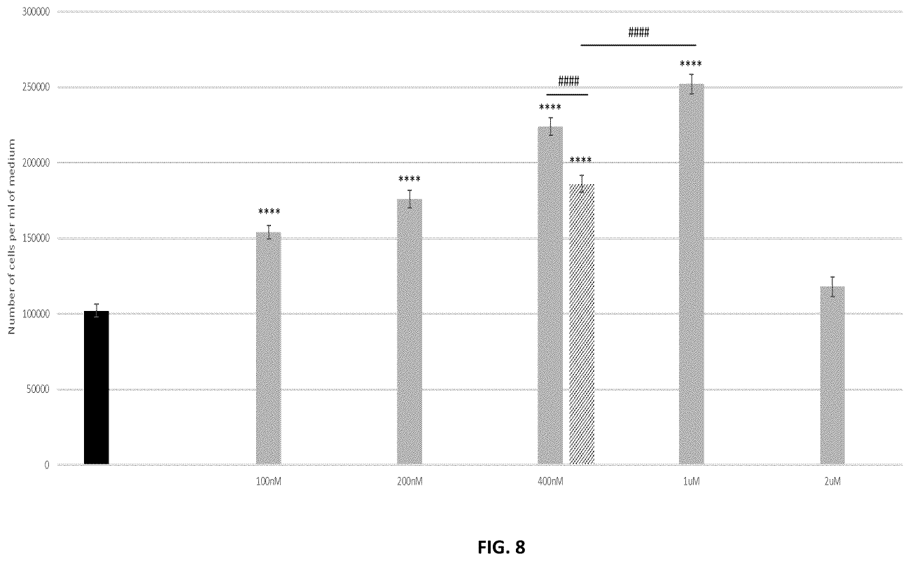

Importantly, stimulating Min6 cells with variant #051 generated a dose-response curve with a similar shape as the one observed upon treatment with variant #047 (FIG. 8). However, variant #051 showed an earlier onset of action (100 nM) and induced a significantly stronger proliferation at both 400 nM and 1p M as compared to native hRspo1 at 400 nM (FIG. 8).

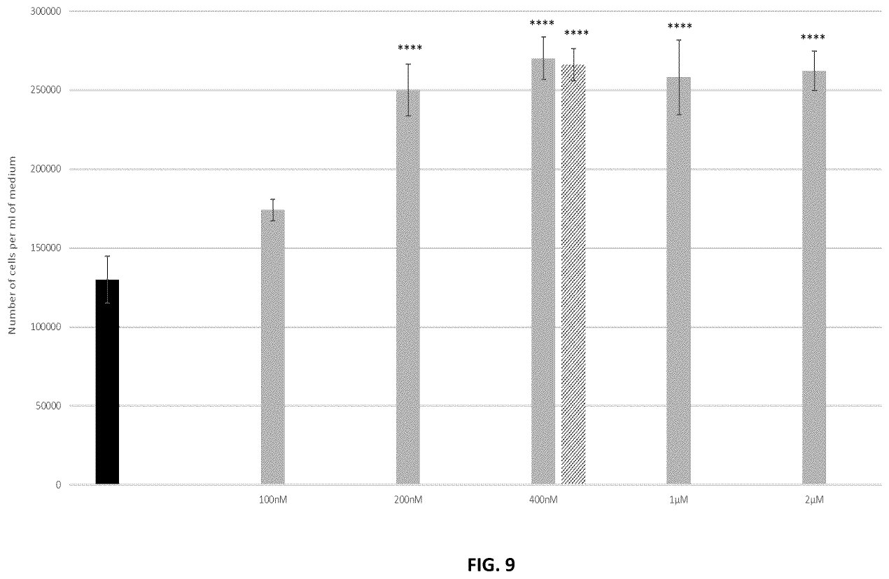

Interestingly, Fc-conjugated hRspo1 variant (#063) was found to be as efficient as the original protein when incubated at a concentration of 400 nM (FIG. 9). However, this proliferative effect did not follow a bell-shaped dose-response curve but remained rather steady at higher doses (FIG. 9).

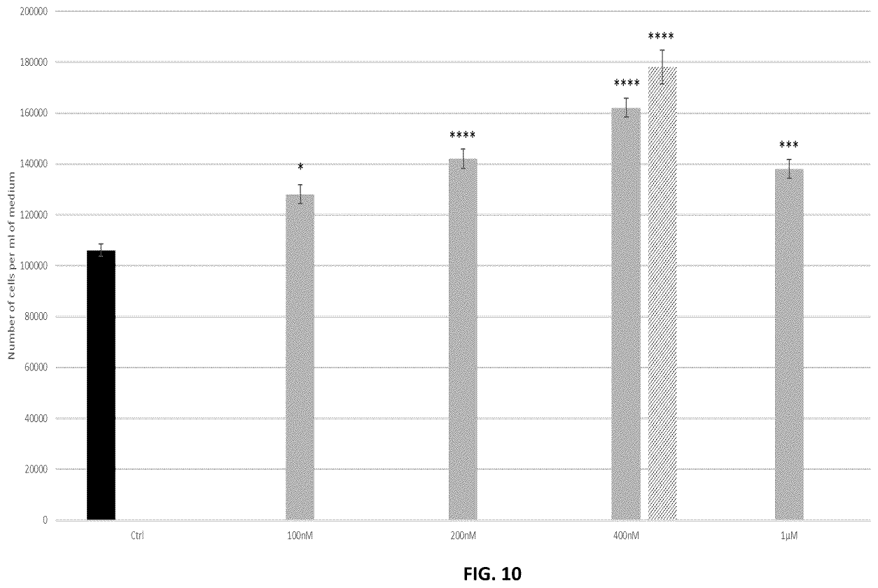

Conversely, N-terminal Fc-conjugated variant #064 did not display any significant proliferative effect on Min6 cells at 400 nM, this protein becoming efficient only when diluted at 1p M or more (FIG. 10).

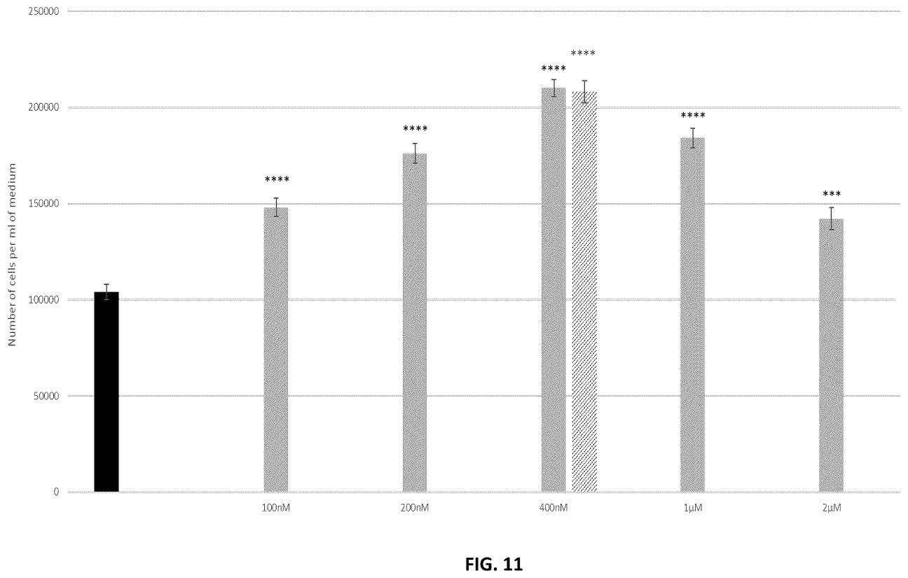

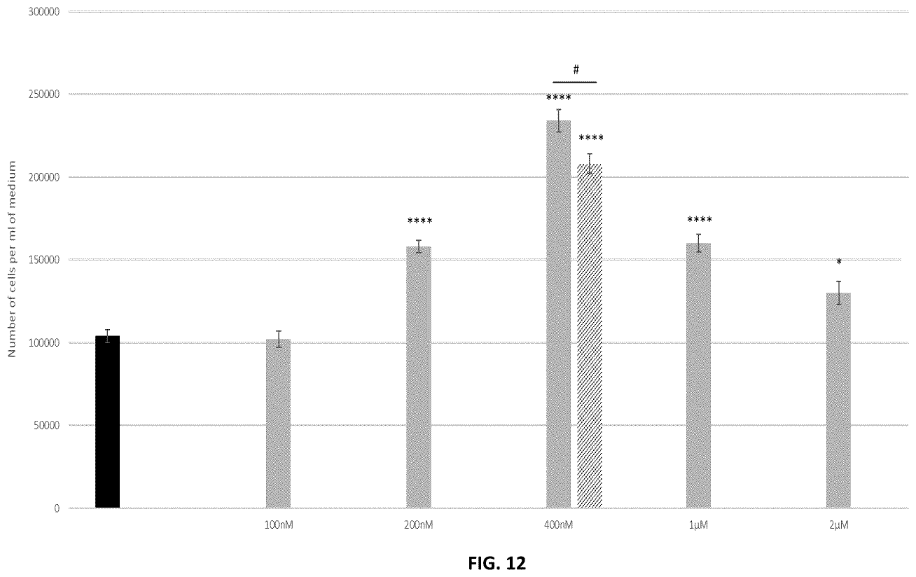

Mitotic effect of variant #049 comprising a tag His and variant #056 created the same bell-shaped dose-response curve and showed the same efficiency as native recombinant hRspo1 (FIG. 11-12).

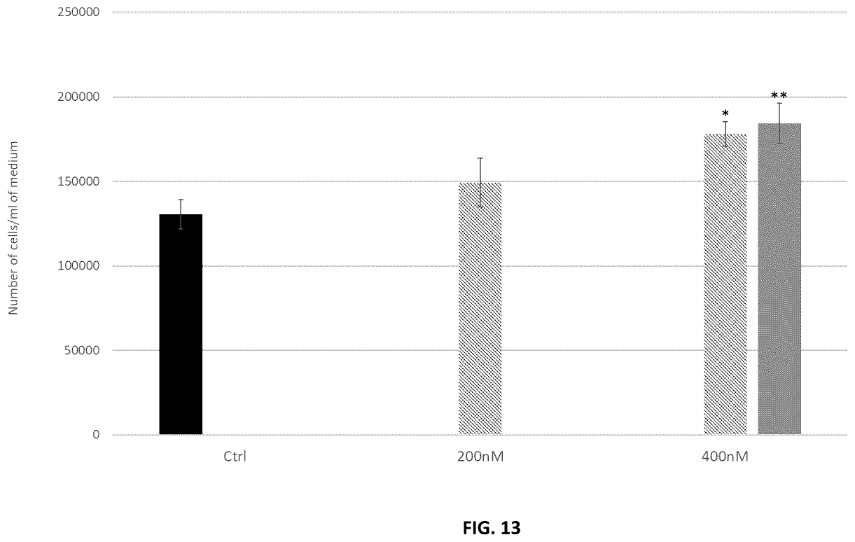

The efficacy of the tagless Variant #049, was also tested in Min6 cells. Min6 cell number significantly increased after 24-hour incubation with Variant #049 at 400 nM (FIG. 13).

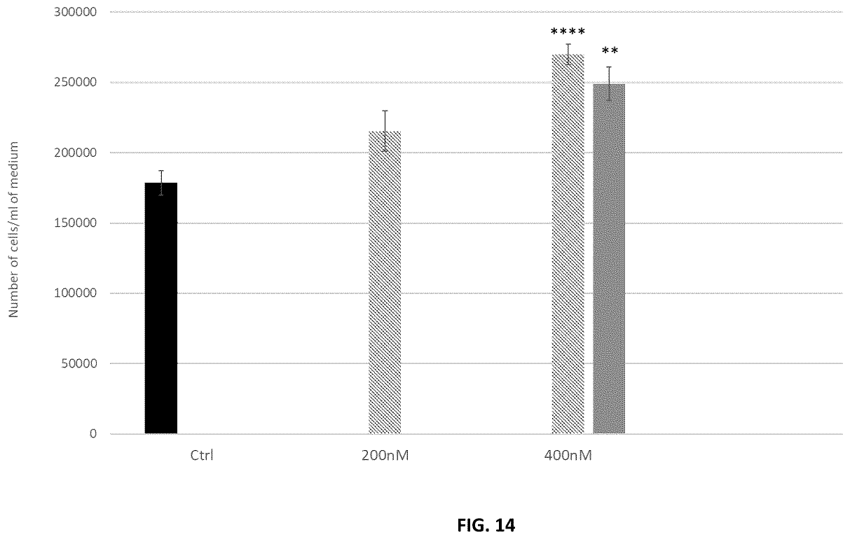

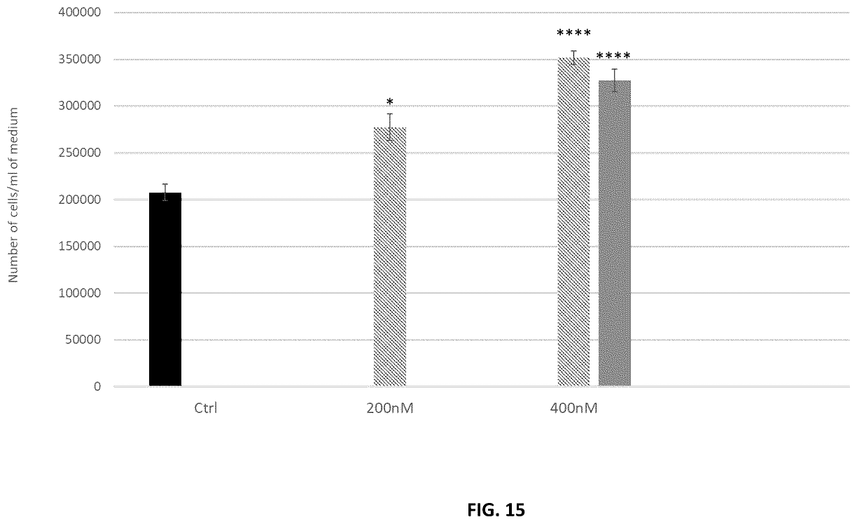

Importantly, this increase was shown to be stronger and more significant when the same protein was incubated for 48h or 72h (FIGS. 14 and 15).

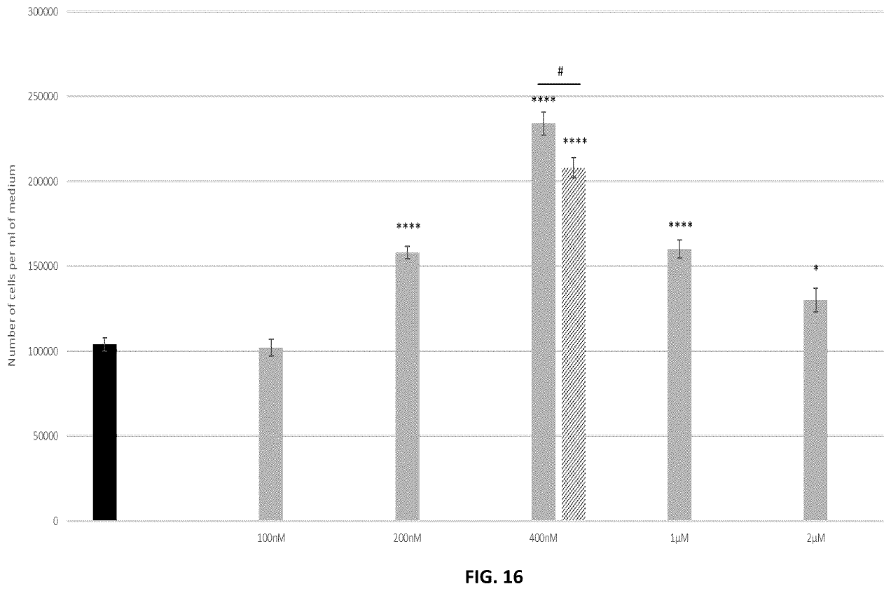

Similarly, variant #054 was observed to be increasingly efficient up to 400 nM dose, this efficiency progressively decreasing at higher concentrations (FIG. 16). However, this analog was found to be significantly more potent than the original hRspo1 in stimulating Min6 proliferation at the peak of its dose-response curve (FIG. 16).

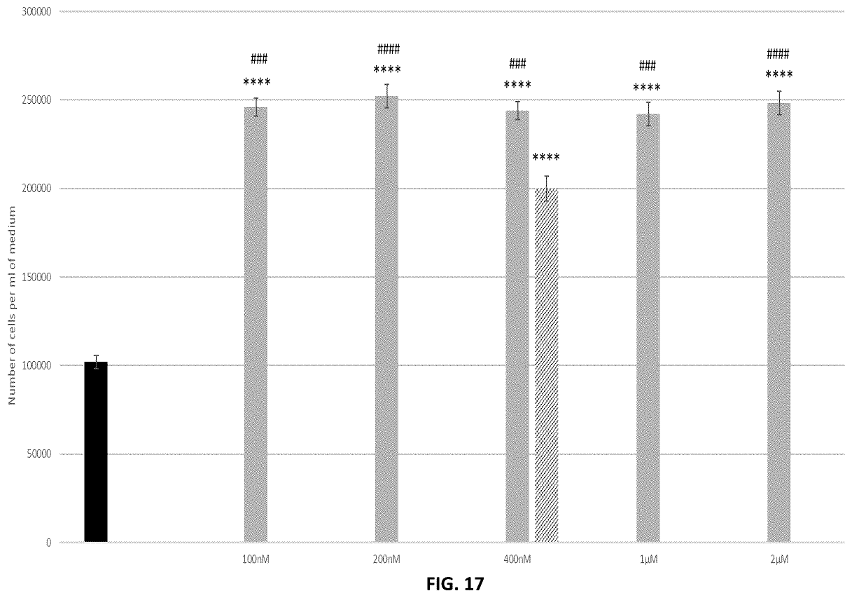

Unexpectedly, variant #082 strongly stimulated Min6 cell proliferation at all concentrations tested, displaying a mitotic activity significantly more powerful than hRspo1, even at lower doses (FIG. 17).

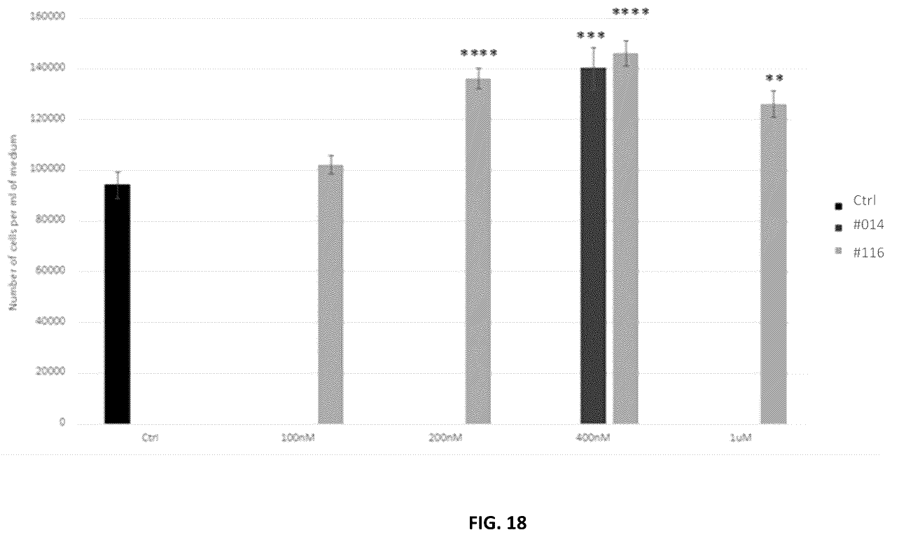

Variant #116 increases Min6 cell number in a bell-shaped fashion with a peak of activity at 400 nM (FIG. 18).

The native human Rspo3 was also tested for their capacity to induce beta-cell proliferation in vivo. Additional analysis then demonstrated that the short-term exposure (5-30 min) of wild-type mice to human Rspo3 also induces beta-cell proliferation in vivo.

D. Determination of ZNRF3 Binding

We produced Variants #009, #047 and #051 and tested them for their capacity to improve binding to ZNRF3 as compared to original native Rspo1, using the ZNRF3-Fc receptor binding assay as described above.

While Variant #009 behaves similarly to our control Rspo1 (without mutation), the Variants #047 and #051 shows superior binding affinity to ZNRF3 as compared to control Rspo1.

2. Beta-Cell Proliferation in Isolated Islets

2.1 Methods

2.1.1 Animals

Male 12952/SvPasCrl mice were obtained from Charles River Laboratories (69210 Saint-Germain Nuelles, France): 60 mice were used at the age of 10 to 12 weeks. All experiments on animals were carried out in accordance with the European animal care guidelines (2010/63/UE) and part of the authorized project No 2796. Animals were acclimatized to the environment for 1 week before the beginning of the experiment and were housed in a temperature-controlled (22 t 2° C.) area and with a 12-hour light-dark cycle (light on at 7.00 am). All mice were allowed to eat normal grow diet A04 from SAFE (Scientific Animal Food and Engineering—Route de Saint Bris—89290 AUGY—France) and drink ad libitum. The litters (sterile sawdust) were changed every other day. The mice were divided into groups of 6 animals per cage. The dimensions of the cage were 42.5×26.6×15.5 cm. General signs were observed and only animals without any abnormal signs were included in the study.

Mice were anesthetized with an intraperitoneal injection of pentobarbital and pancreases were perfused with collagenase to further isolate islets. After an overnight stabilization period in RPMI 1640, islets were dispatched and treated for 72h with compound and reference to further proceed to cell proliferation evaluation.

2.1.2 Islet Isolation and Treatment

Islets from 129/Sv mouse were isolated by collagenase digestion of the pancreas and washed in Hanks Balanced Salt solution (HBSS) followed by purification on density gradient using Histopaque 1077 and HBSS. Islets were dispatched by handpicking at a density of 30 islets into Petri dishes and placed in RPM11640 supplemented with 10 mM Hepes, 2 mM Glutamine, 100U/ml Penicillin, 100 μg/ml Streptomycin and SVF 10% at 37° C. in a humidified atmosphere of 90% air/5% CO2. After an overnight stabilization period, culture medium was removed and fresh culture medium without (Control) or with tested compound or reference was added. Then medium and treatments were renewed 24h and 48h later. For the last 24h of treatment, BrdU (10 μM) was added to the culture medium.

The islets were divided into 8 groups, with a number of Petri dishes of 6 per group, as described in the following Table 4:

| Group | Treatment | Number of islets |
|---|---|---|
| 1 | Control | 180 in 6 Petri dishes |
| 2 | #14 0.2 μM | 150 in 5 Petri dishes |
| 3 | #14 1 μM | 150 in 5 Petri dishes |
| 4 | #14 3 μM | 150 in 5 Petri dishes |
| 5 | #64 0.2 μM | 150 in 5 Petri dishes |
| 6 | #64 1 μM | 150 in 5 Petri dishes |
| 7 | #64 3 μM | 150 in 5 Petri dishes |
| 8 | #51 0.2 μM | 150 in 5 Petri dishes |
| 9 | #51 1 μM | 150 in 5 Petri dishes |
| 10 | #51 3 μM | 150 in 5 Petri dishes |
| 11 | #008 0.2 μM | 150 in 5 Petri dishes |
| 12 | #008 1 μM | 150 in 5 Petri dishes |
| 13 | #008 3 μM | 150 in 5 Petri dishes |
| 14 | #49 0.1 μM | 150 in 5 Petri dishes |
| 15 | #49 0.2 μM | 150 in 5 Petri dishes |
| 16 | #49 0.4 μM | 150 in 5 Petri dishes |
| 17 | #49 1 μM | 150 in 5 Petri dishes |
| 18 | #49 2 μM | 150 in 5 Petri dishes |
| 19 | #121 0.1 μM | 150 in 5 Petri dishes |
| 20 | #121 0.2 μM | 150 in 5 Petri dishes |
| 21 | #121 0.4 μM | 150 in 5 Petri dishes |
| 22 | #121 1 μM | 150 in 5 Petri dishes |
| 23 | #121 3 μM | 150 in 5 Petri dishes |
| 24 | GLP-1 - 1 nM | 180 in 6 Petri dishes |

2.1.3 Preparation of Isolated Islets for Proliferation Measurement

After 72h of treatment, batches of 30 islets were collected from Petri dishes for each condition and transferred into 1.5 ml Eppendorf tubes. Then, islets were washed 3 times with DPBS by centrnfugation (1000 rpm, 30 sec, 4° C.) before to be digested by Trypsin-EDTA 0.25%. The reaction was stopped by adding RPMI 1640 and digested islets were cytospined on cytoslide.

Islets were then fixed with paraformaldehyde 3.7% during 30 min and permeabilized with Triton X100—0.2% in BSA 5% and antigen retrieval was performed using citrate buffer at pH 6 at 100° C. for 20 min. To prevent non-specific binding of antibodies, a blocking step in PBS-5% BSA was performed before immunostaining.

2.1.4 Determination of Beta-Cell Proliferation

Cell proliferation was estimated by measurement of BrdU positive cells in sections after double immunostaining with a rat anti-BrdU antibody (Abcam—Ref. ab6326) coupled with goat anti-rat IgG Alexa Fluor 647 (ThermoFisher Scientific—ref. A-21247) and a mouse anti-insulin antibody (Sigma, ref. 12018) coupled with a goat anti-mouse IgG DyLight® 488 antibody (Diagomix—ref. GtxMu-003-D488NHSX). Cell nuclei were stained using ProLong™ Gold Antifade Mountant with DAPI (Life Technologies—ref. P36935). The number of cells stained with BrdU immunostaining was determined after slide scanning and analysis with NDP view or case viewer imaging software. The analysis was performed on 5 to 6 samples per batch.

2.2 Results

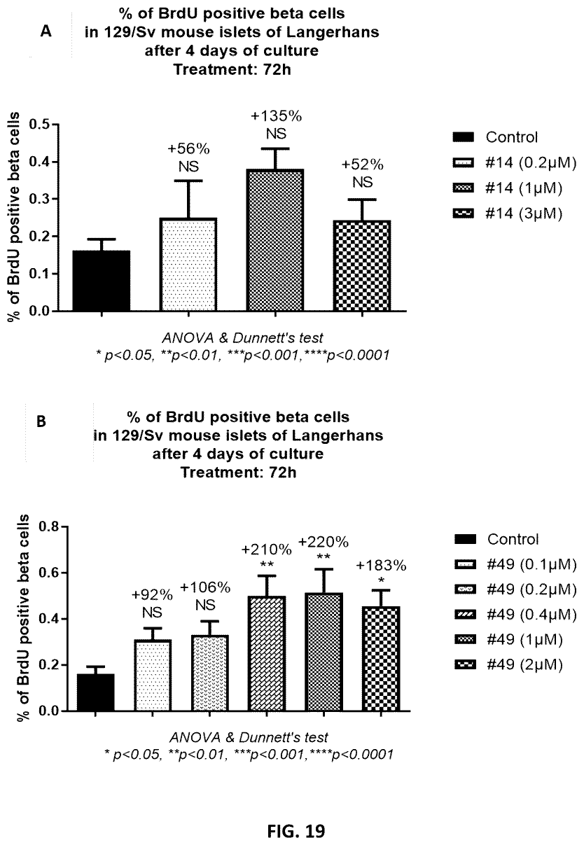

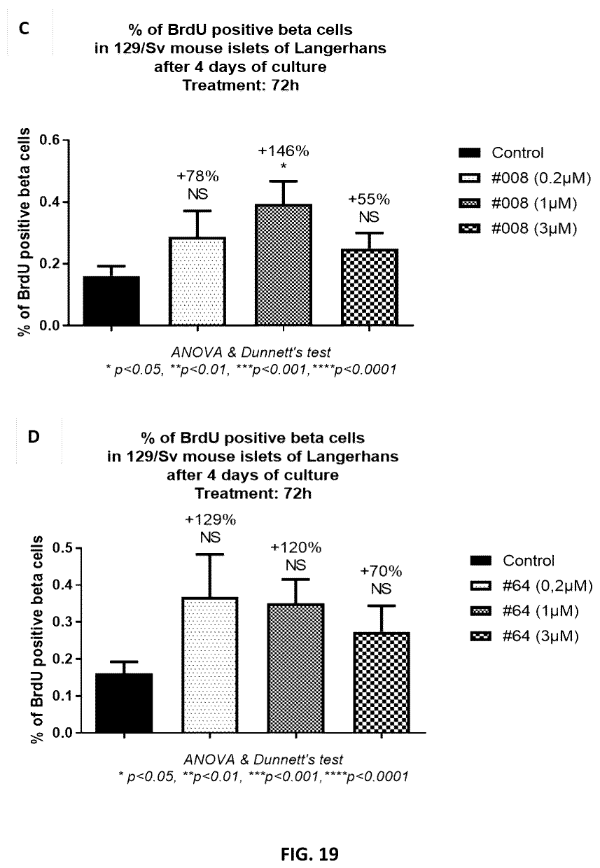

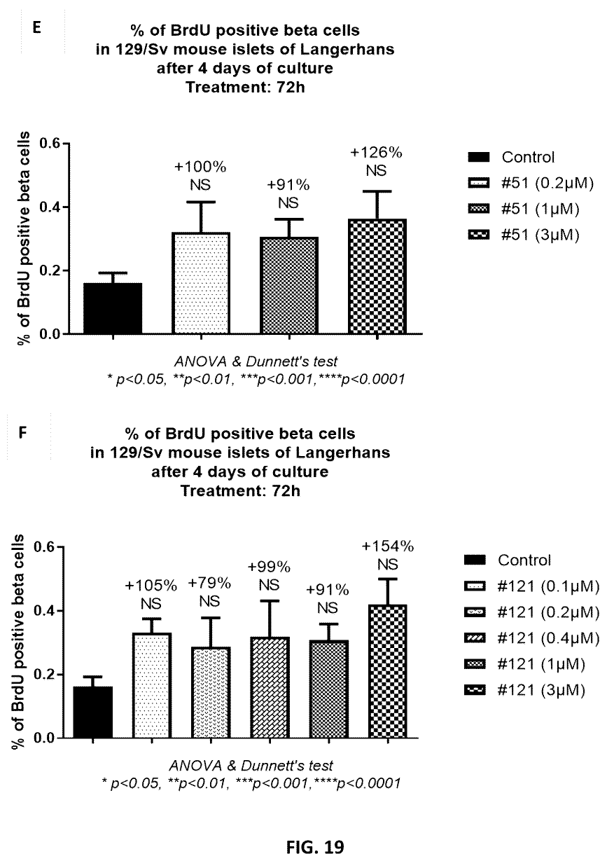

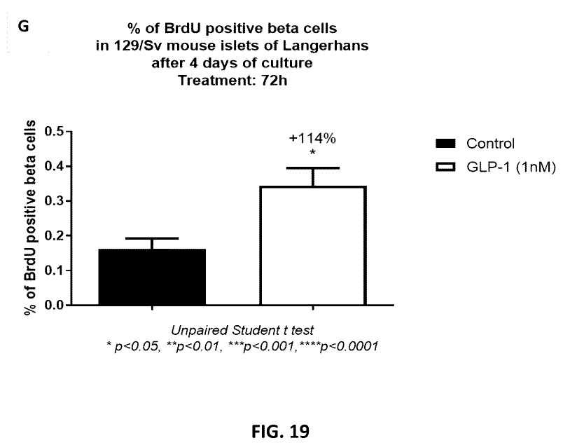

The study was designed to evaluate the effect of a 72h-exposure to six variants: #008, #014, #049, #051, #064 and #121 on beta-cell proliferation in 129/Sv mouse islets of Langerhans. #008, #014, #051, #064 proteins were tested at 3 concentrations (0.2, 1 and 3 μM) and #049 and #121 at 5 concentrations (0.1, 0.2, 0.4, 1 and 2 μM (#049) or 3 μM (#121)). Cell proliferation was calculated by dividing the number of BrdU positive cells by the total number of cells and expressed as % (FIG. 19). Results were expressed as the mean t SEM. The number of observations was 5 to 6 per treatment. To analyze compound effects, statistical analyses were performed using an Anova followed by a Dunnett's test or a Kruskal-Wallis followed by a Dunn's test when variances significantly differ (GraphPad PRISM08). A p value of <0.05 was considered as significant.

3. Glycemia Evolution and Beta-Cell Mass in Female NOD Mice

3.1 Methods

3.1.1 Animals

Female NOD/Mrk Tac mice were provided by Taconic Biosciences (4623 Ejby, Lille Skensved, Denmark): 55 mice were 6 to 8-week-old when transferred to CNRS—Universitë de Paris—UMR 7592 animal facility. All experiments on animals were carried out in accordance with the European animal care guidelines (2010/63/UE) and part of the authorized project #20173 and #31876. Animals were acclimatized to the environment for 1 week before the experiment and were housed in a temperature-controlled (22 t 2° C.) area and with a 12-hour light-dark cycle (light on at 7.00 am). All mice were allowed to eat NIH-31M diet from ALTROMIN (Altromin Spezialfutter GmbH & Co. KG—Im Seelenkamp 20—D-32791 Lage—Germany) and drink ad libitum. The litters (sterile sawdust) were changed every other day. The mice were divided into groups of 5 animals per cage. The dimensions of the cage were 37.3×23.4×14.0 cm. General signs were observed and only animals without any abnormal signs were included in the study.

3.1.2 Study Design

During the 16 weeks of the study, proteins were delivered to mice by intraperitoneal route once a day for variant #014 and once a week for Variant #064 in the morning at the dose provided by the sponsor with an administration volume of 10 mV/kg as described in Table 5 below:

| | Intraperitoneal | | Number |
|---|---|---|---|
| Compound | dose μg/kg | Frequency | of mice |
| NOD controls | Vehicle | Daily | 15 |
| #064 | 2400 | Weekly | 14 |
| #014 | 400 | Daily | 13 |
| #014 | 800 | Daily | 13 |

Drug solutions were prepared as follows:
#064: the appropriate volume of the protein at 1 mg/ml in PBS1× pH7.4 was diluted in PBS1× pH 7.4 to obtain a 240 μg/ml solution (2400 μg/kg/10 ml).
#014: the appropriate volume of the protein at 1 mg/ml in PBS1× pH7.4 was diluted in PBS1× pH 7.4 to obtain a 80 μg/ml solution (800 μg/kg/10 ml).

This solution was further diluted 1:2 in PBS1× pH 7.4 to obtain a 40 μg/ml solution (400 μg/kg/10 ml). Volumes to be prepared were adjusted to the number of animals to be injected.

Between week 1 and week 18, all animals received the vehicle or the compound once daily in the morning by intraperitoneal route (once a week for #64). Glycemia was monitored once a week until day week 16 using an Accu-Check reader after blood sampling at the tail vein.

Ten-week-old female NOD mice were daily intraperitoneally injected with tagless Variant #049 for 13 weeks at a concentration of 0.2 mg/Kg, 0.4 mg/Kg and 0.8 mg/Kg. Their glycemia and body weight was weekly monitored. Mice were considered diabetic if blood glucose levels exceeded 250 mg/dl.

Analytic methods

Glucose concentration is determined with a commercially available kit from Horiba Medical (ref. A11A01667). The procedure is based on a two-phase enzymatic reaction:

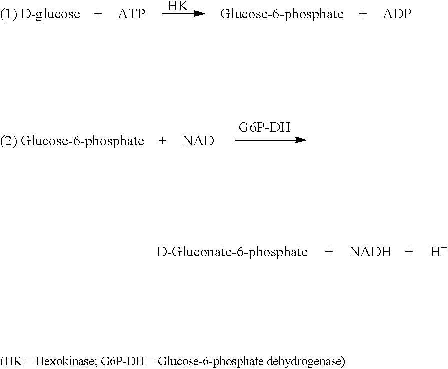

The reaction is monitored kinetically by measuring the absorption caused by the NADH produced in the second phase. The glucose content of the sample can be calculated from the measured change of absorbance.

Insulin concentration is determined with a commercially available kit from Alpco (ref. 80-INSMSU-E01/E10). The ALPCO Mouse Ultrasensitive Insulin ELISA is a sandwich type immunoassay. The 96-well microplate is coated with a monoclonal antibody specific for insulin. The standards, controls, and samples are added to the microplate wells with the conjugate. The microplate is then incubated on a microplate shaker at 700-900 rpm. After the first incubation is complete, the wells are washed with Wash Buffer and blotted dry. TMB Substrate is added, and the microplate is incubated a second time on a microplate shaker at 700-900 rpm. Once the second incubation is complete, Stop Solution is added, and the optical density (OD) is measured by a spectrophotometer at 450 nm. The intensity of the color generated is directly proportional to the amount of insulin in the sample. The measurement range is between 0.025-6.9 ng/mL.

Concomitantly, NOD animals were intraperitoneally injected with #121 (Fc fragment fused at the N-terminal of #049) weekly at a concentration of 2400 μg/Kg for 13 consecutive weeks. These experiments aimed to assess whether the weekly administration of the Fc-fused protein #049 was sufficient to prevent, counteract or delay diabetes onset in vivo. At the end of the experiment, mice administered with #049 (with tag HA) and #121 were sacrificed by cervical dislocation. Pancreata were collected and fixed in Antigenfix (paraformaldehyde solution pH 7.2-7.4; Microm Microtech France), washed in cold PBS and incubated 1 hour in 0.86% saline. Following dehydration through a series of increasing ethanol dilutions (50%, 70%, 80%, 90%, and 100%), the pancreata were treated with isopropanol and toluene and embedded in paraffin. Paraffin blocks were sectioned into 6 μm slides, analyzed by immunofluorescence using antibodies against insulin and glucagon and mounted with a medium containing DAPI for nuclear counterstaining. Total beta-cell mass was assessed on 5-6 slides randomly selected throughout the entire organ. Acquisition of whole slide images was achieved using a whole slide scanner and insulin immunosignal quantification on mosaic images was obtained using the HALO/Indica Lab Image Analysis Platform for image quantification.

3.2 Results

The study is designed to evaluate the effect a 18-week treatment by intraperitoneal route with #014 protein at 2 doses (400 & 800 μg/kg) and #64 protein at one dose (2400 μg/kg) on glycemia evolution and diabetes phenotyping and beta-cell mass in female NOD mice (4 groups): vehicle (n=15), #014 protein at 2 concentrations (n=13), #64 protein at one concentration (n=14), compound administration for 18 weeks from 10 weeks of age.

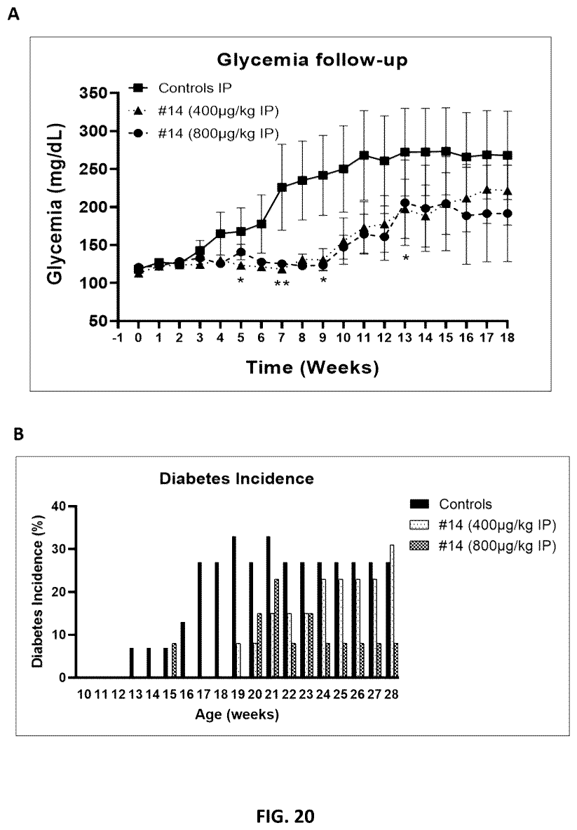

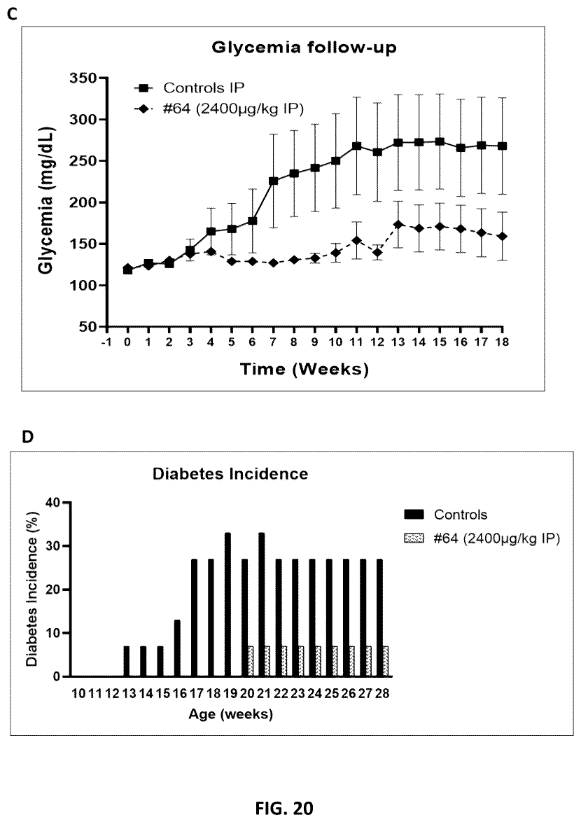

Results in FIG. 20 were expressed as the mean t SEM together with number of individual observations (n). Statistical analysis was performed by using an Anova followed by a Dunnett's t test to compare treated groups to the STZ control group. A Kruskal-Wallis test followed by a Dunn's test was used if variances between groups are significantly different as calculated by the Bartlett test (GraphPad PRISM08). A p value of 0.05 was considered as significant.

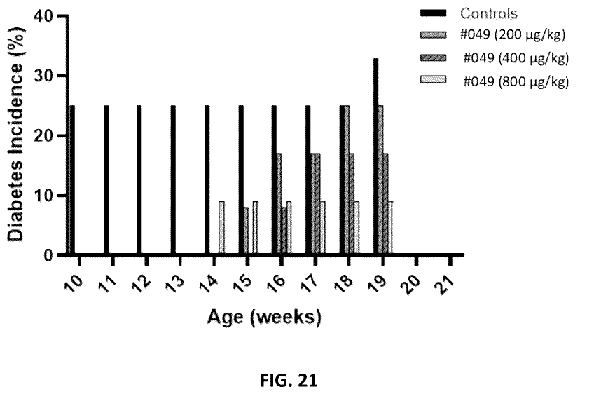

To assess the ability of Variant #049 to counteract p-cell loss in a diabetic context, the inventors used NOD mice as type 1 diabetes model. Interestingly, daily intraperitoneal administration of Variant #049 dose-dependently reduced diabetes incidence (FIG. 21).

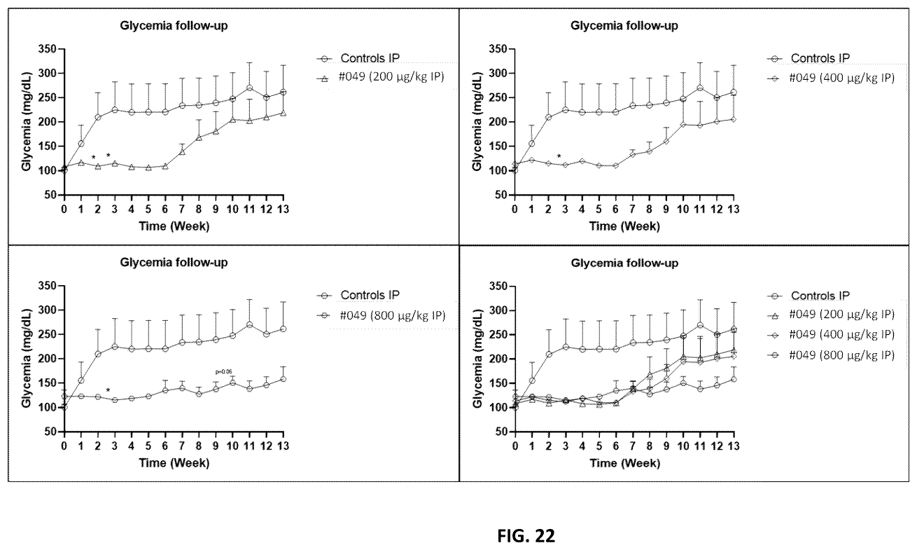

Importantly, rodents treated with Variant #049 at a concentration of 0.8 mg/Kg displayed a negligible increase in basal glycemia as compared to controls (FIG. 22).

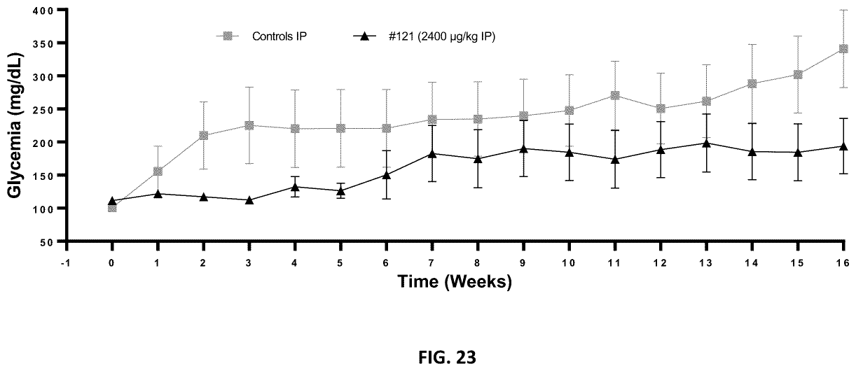

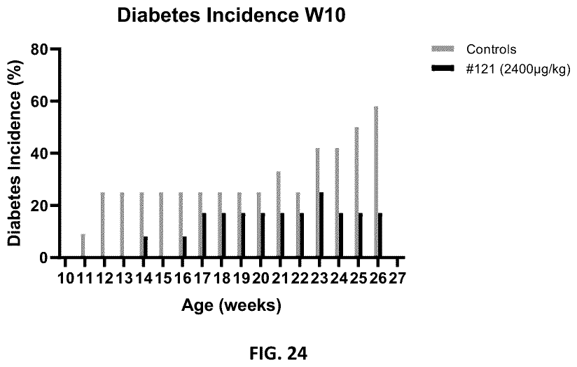

Consistently with results obtained with protein #049, mice treated with #121 weekly displayed lower glycemia as compared to PBS-injected controls. At the end of treatment, glycemia was decreased by 36% in #121-treated mice compared to control animals (FIG. 23). Also, #121 administration delayed diabetes onset and reduced diabetes incidence by 63% compared to controls (FIG. 24).

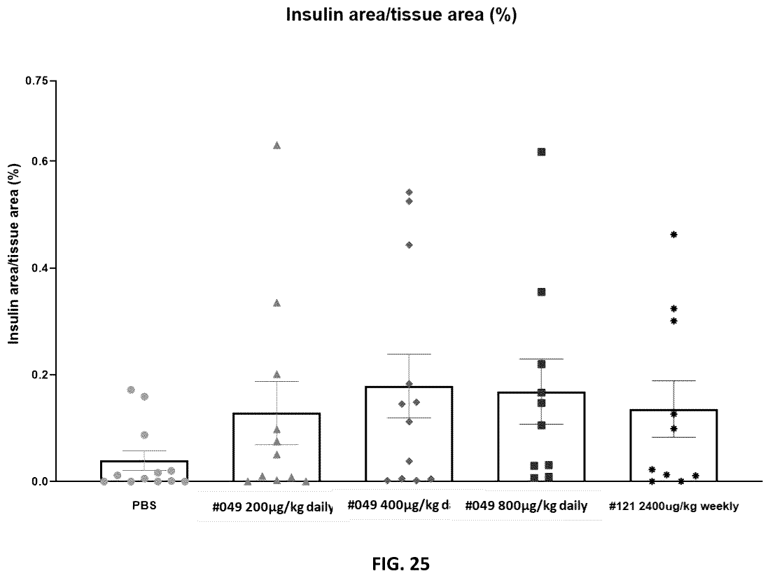

At the end of the experiment, immunofluorescence quantitative analyses showed an increased in beta-cell mass in animals treated with both #049 with tag HA and #121, coherently with the lower glycemia levels previously observed (FIG. 25). Specifically, beta-cell area normalized by tissue area was increased respectively by 3.3-4.5- and 4.3-fold in mice treated daily with #049 at 200 μg/Kg, 400 μg/Kg and 800 μg/Kg and by 3.4-fold in mice treated weekly with #121 at 2400 μg/Kg (FIG. 25).

4. In Vivo Tests

4.1 Methods

4.1.1 In Vivo Mid-Term Proliferation Assay

In vivo proliferation tests are performed on 2 months-old wild-type male 129SV mice. Following a minimum acclimatation period of 3 days, animals are daily injected intraperitoneally for 5 consecutive days with 0.4 mg/Kg and 0.8 mg/kg of #014. Mice treated with daily injections of 150 ul of sterile PBS are used as controls. Additionally, all mice were provided with the thymidine analog bromodeoxyuridine (BrdU) diluted in the drinking water at a concentration of 1 mg/ml for 72 hours before sacrifice. Finally, mice are sacrificed 30 minutes after last injection by cervical dislocation. Pancreata are collected and fixed in Antigenfix (paraformaldehyde solution pH 7.2-7.4; Microm Microtech France), washed in cold PBS and incubated 1 hour in 0.86% saline. Following dehydration through a series of increasing ethanol dilutions (50%, 70%, 80%, 90%, and 100%), the pancreata are treated with isopropanol and toluene and embedded in paraffin. Paraffin blocks are sectioned into 6 μm slides and analyzed by immunofluorescence using antibodies against insulin, Ki67 and BrdU.

4.4.2 In Vivo Efficacy Test on Streptozotocin-Induced Mouse Hyperglycemic Model

These tests are performed on adult 2 months-old wild-type male 129SV mice. To induce hyperglycemia 129SV male mice are intraperitoneally injected with streptozotocin (STZ), a glucosamine-nitrosourea compound, displaying cytotoxic effects, resulting from DNA and chromosomal damage. Briefly, STZ is dissolved in 0.1M sodium citrate buffer (pH 4.5) and administered at a dose of 50 mg/Kg over 3 consecutive days, after 5 hours of starvation.

Hyperglycemia development was assessed by monitoring the blood glucose levels with a ONETOUCH glucometer (Life Scan, Inc., CA). Water consumption was manually determined once a week. To evaluate glucose tolerance, an intraperitoneal glucose tolerance test (ip-GTT) was performed. For these tests, mice were fasted for 5 hours and injected intraperitoneally with 2 g/kg of bodyweight of D-(+)-glucose. Blood glucose levels were measured at the indicated time points post-injection with a ONETOUCH glucometer. PBS or variant #014 (SEQ ID NO: 41) was daily administered by intraperitoneal injection at a dose of 0.8 mg/Kg, starting either 15 days before or 7 after STZ treatment.

4.4.3 In Vivo Efficacy Test on Transplanted Human Islets

For this experiment, 12 Rag2N12 immunodeficient mice were transplanted with human islets (500 islets-equivalent/mouse). After gas anesthesia (isoflurane), a lumbar laparotomy was performed to access the left kidney. Islet pellet (500 Islets-equivalent) was injected through a catheter in the subcapsular space of the kidney of each animal. After the graft, capsule was cauterized to avoid hemorrhage and cell leakage. Suturing was done on both muscular and skin layers. Subcutaneous morphine (Buprenorphine—0.05 mg/kg) was administered to palliate perioperative pain. Each mouse was housed in sterile cages on a SOPF animal facility. Following a recovery of 2 weeks, mice were daily treated with PBS or variant #14 at a dose of 0.4 mg/Kg and 0.8 mg/Kg for 60 days. Every 2 weeks, blood c-peptide levels were measured following 6 hours of starvation. After 28 days of variant #014 treatment glucose handling was assessed by ip-GTT. Finally, animals were provided with BrdU diluted in the drinking water at a concentration of 1 mg/ml for 1 week before sacrifice. At the end of the experiment, mice were sacrificed and islets grafts were isolated, fixed and analyzed by immunofluorescence using antibodies against insulin and BrdU.

4.2 Results

4.2.1 Assessment of hRspo1 Proliferative Activity In Vivo

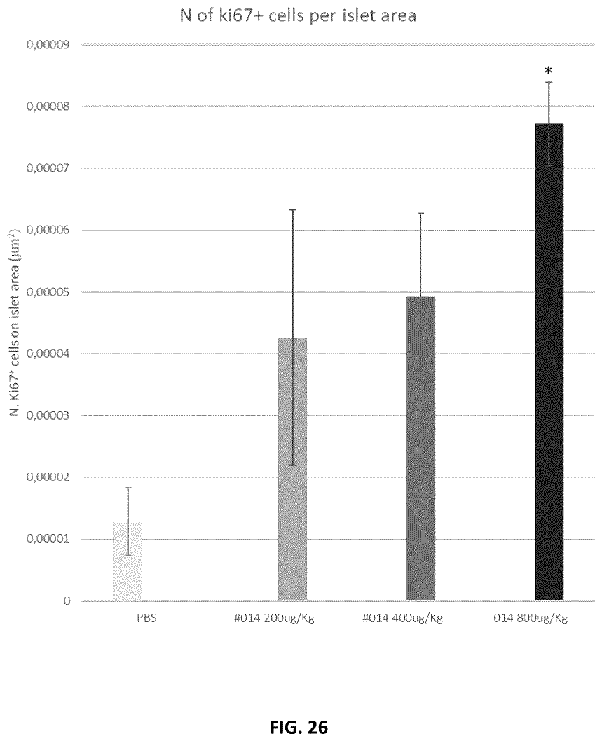

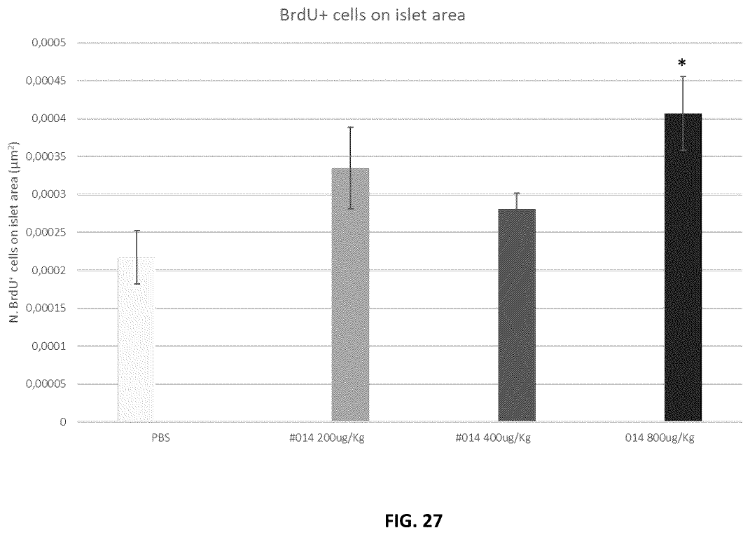

To evaluate the ability of hRspo1 to induce adult murine β-cell neogenesis, the inventors administered variant #014 intraperitoneally for 5 consecutive days. Importantly, an increase in cells co-expressing insulin and the endogenous nuclear proliferative marker Ki67 was observed in pancreatic samples of mice treated with variant #014 at all doses tested (FIG. 26). Notably, a significant 6-fold increase was assessed in tissues isolated from animals treated with 0.8 mg/Kg of variant #014. Accordingly, variant #014 administration was shown to enhance BrdU accumulation within pancreatic β-cells (FIG. 27). Yet again, BrdU/insulin co-expressing cells were significantly more abundant in pancreatic sections of mice injected with 0.8 mg/Kg of variant #014.

4.2.2 Effect of Variant #014 on Glycemic Control in STZ-Induced Hyperglycemic Mouse Model

This study was designed to evaluate the effect of a chronic treatment with #014 protein (0.8 mg/kg) on glycemic control in a hyperglycemic mouse model induced by multiple low-doses of streptozotocin (STZ). Hyperglycemia was induced with 3 doses of STZ (50 mg/Kg) over 3 consecutive days. Variant #014 or vehicle were administered daily by intraperitoneal injection starting from either 15 days before or 7 days after STZ treatment.

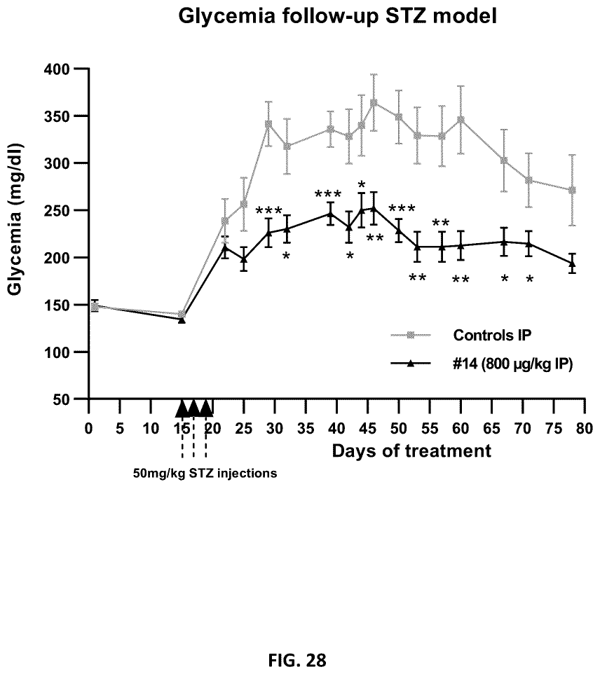

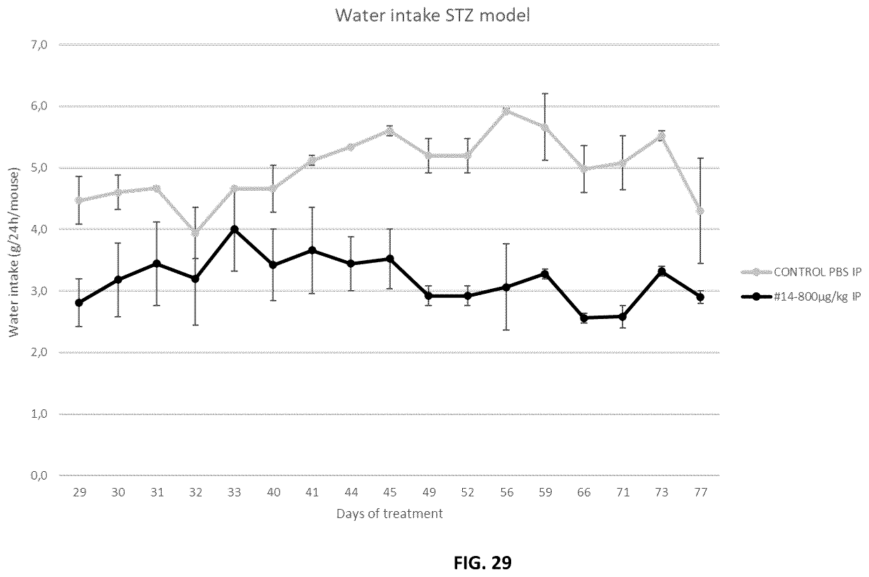

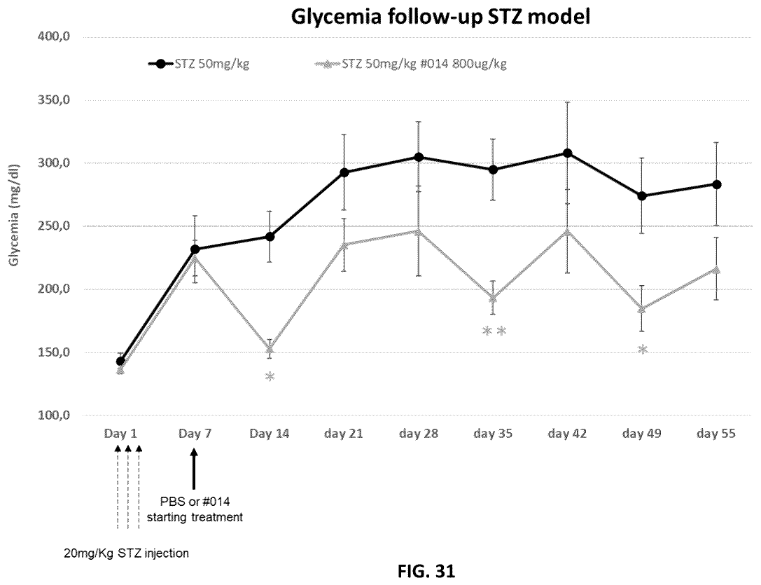

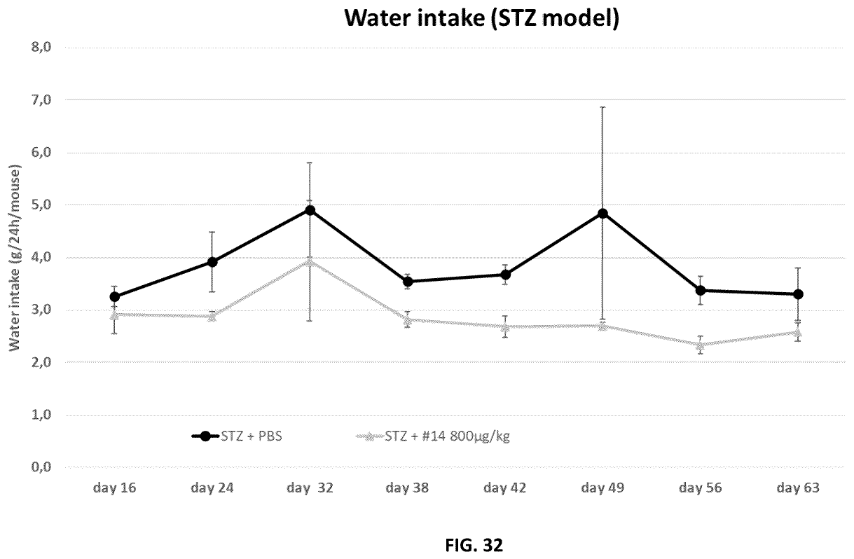

In both experimental settings, STZ efficiently induced hyperglycemia in all the animals. However, blood glucose levels of mice injected with variant #014 remained steadily lower as compared to vehicle-injected controls throughout the entire experimental period of 2 months (FIGS. 28 and 31). Importantly, this difference was more striking and significant when variant #014 was inoculated before STZ-mediated hyperglycemia development. Accordingly, 24h-water intake was consistently decreased in mice treated with variant #014, indicating that chronic administration of hRspo1 alleviates hyperglycemia-associated polydipsia (FIGS. 29 and 32).

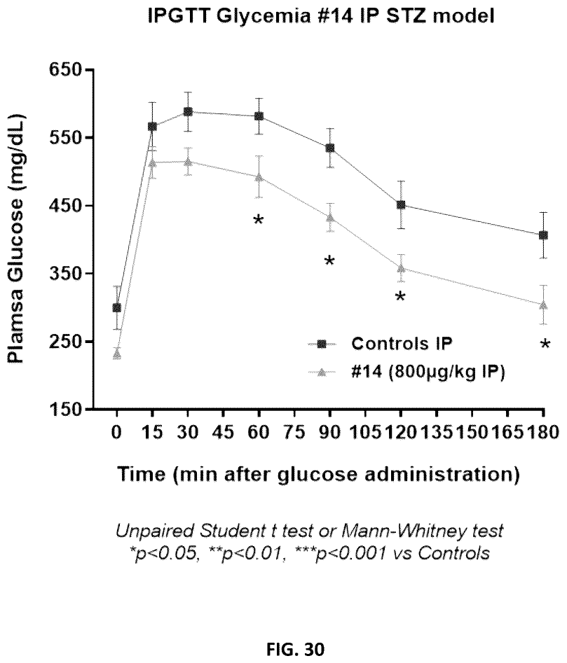

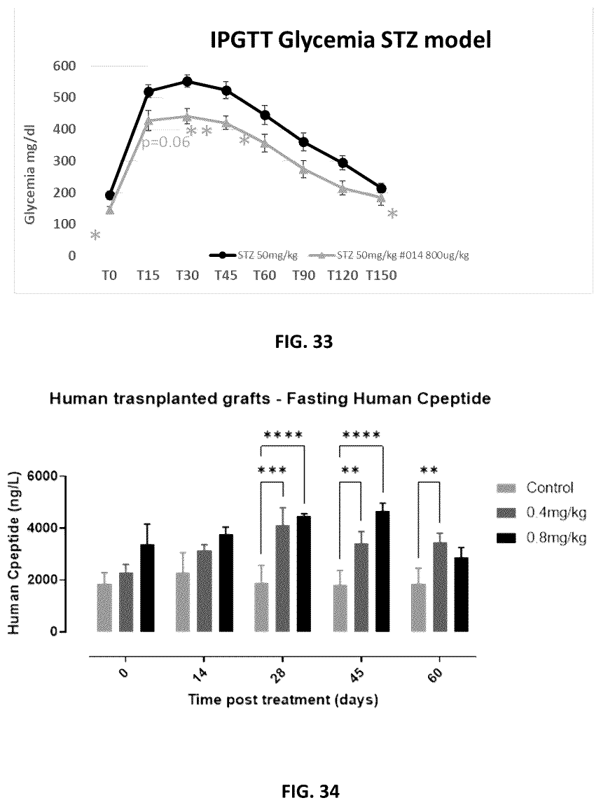

Following 2 months of glycemia monitoring, an ip-GTT revealed that glucose tolerance was significantly improved in the mice treated IP with #14 protein as compared to controls, in both experiments (FIGS. 30 and 33). These data indicate that variant #014 is able to improve glycemic control in chronic hyperglycemic mouse models.

4.2.3 Effect of Variant #014 on Grafted Human Islets

To test the proliferative activity of variant #014 on human β-cells, this molecule was daily injected in immunodeficient mice transplanted with human islets under the kidney capsule.

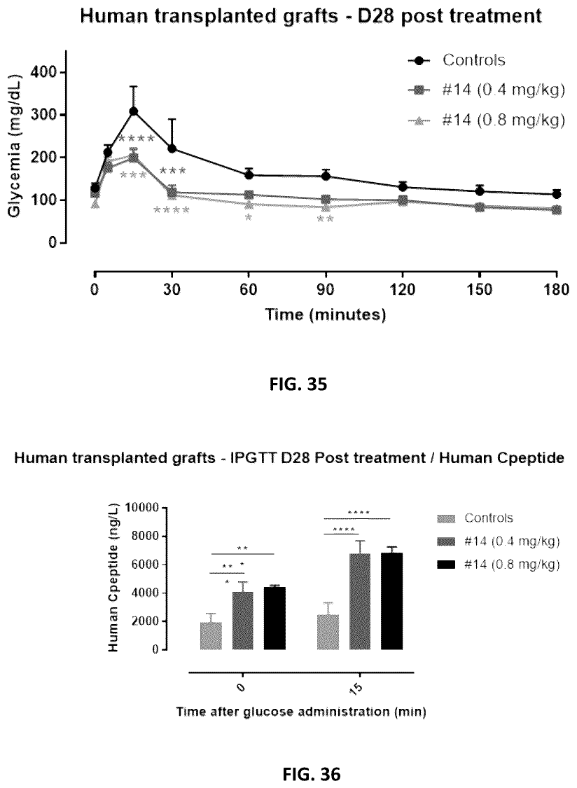

Intriguingly, dosing mice at 0.4 mg/Kg and 0.8 mg/Kg of variant #014 led to a progressive increase in fasting blood human peptide levels as compared to PBS-injected controls (FIG. 34). Of note, this increase became significant at day 45 in mice treated with both doses of variant #014 and remained significant in the experimental group of animals injected with 0.4 mg/Kg at day 60 (FIG. 34). Furthermore, at day 28 glucose tolerance as assessed by ip-GTT was shown to be improved in variant #014-treated animals compared to controls (FIG. 35). Accordingly, plasmatic human c-peptide was found significantly augmented not only upon starvation, but also 15 minutes after stimulation with a glucose bolus (FIG. 36).

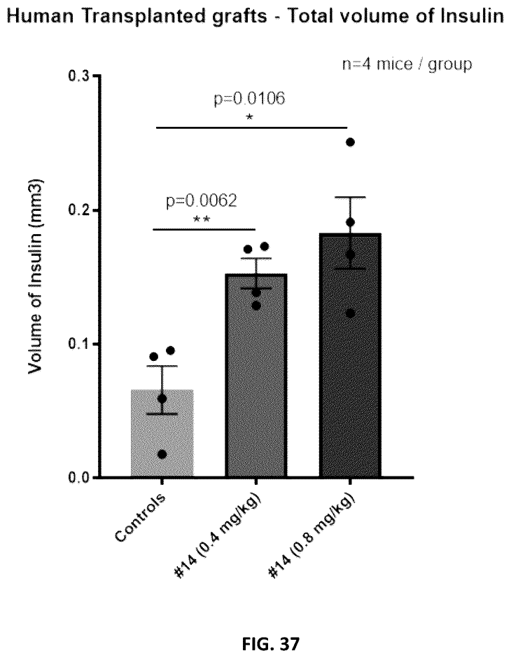

Finally, mice were provided with BrdU in drinking water for a week and sacrificed at day 60, for pancreatic tissue analysis. These studies revealed a strong and significant increase in BrdU-immunolabeled β-cells and β-cell volume in pancreatic sections of rodents injected with variant #014 compared to controls (FIG. 37). Altogether, these data suggest that chronic administration of variant #014 efficiently stimulates human β-cell proliferation and hyperplasia.

5. Pharmacokinetics

5.1 Methods

The pharmacokinetic profiles of the Fc-fused variants #63-1, #63-2 and #64 were investigated upon a single subcutaneous injection to 129/Sv mice at 0.8 mg/kg.

The proteins were administered to mice by the subcutaneous route (SC, skin along the back), with an administration volume of 10 ml/kg, as indicated in Table 6 below:

| | | Protein | | | Time-point | Number |
|---|---|---|---|---|---|---|
| | | Concen- | | Injec- | & pancreas | of |
| | Test | tration | Dose | tion | collection | animals/ |
| Group | item | Provided | (mg/kg) | Route | at 168 h | group |
| 1 | #063-1 | 0.9 mg/ml | 0.8 | SC | 1 h-6 h-72 h- | 3 |
| | | | | | 168 h | |
| | | | | | 3 h-24 h-48 h- | 3 |
| | | | | | 96 h | |
| 2 | #063-2 | 0.9 mg/ml | 0.8 | SC | 1 h-6 h-72 h- | 3 |
| | | | | | 168 h | |
| | | | | | 3 h-24 h-48 h- | 3 |
| | | | | | 96 h | |
| 3 | #064 | 1.0 mg/ml | 0.8 | SC | 1 h-6 h-72 h- | 3 |
| | | | | | 168 h | |
| | | | | | 3 h-24 h-48 h- | 3 |
| | | | | | 96 h | |

Blood samples (3 time-points+1 terminal blood sampling per animal) were collected at 1h, 3h, 6h, 24h, 48h, 72h, 96h and 168h post-dose, as detailed in the table above, and the plasma was isolated for bioanalytical testing.

The concentrations of proteins #63-1, #63-2 and #64 were quantified in plasma using an optimized bioanalytical method, based on a commercial Human R-Spondin 1 DuoSet ELISA kit (R&D Systems).

Concentration versus time plots were generated in Excel the PK data were evaluated using PK Solver add-in for Excel (Zhang Y, t al. Comput Methods Programs Biomed. 2010 September; 99(3):306-14).

5.2 Results

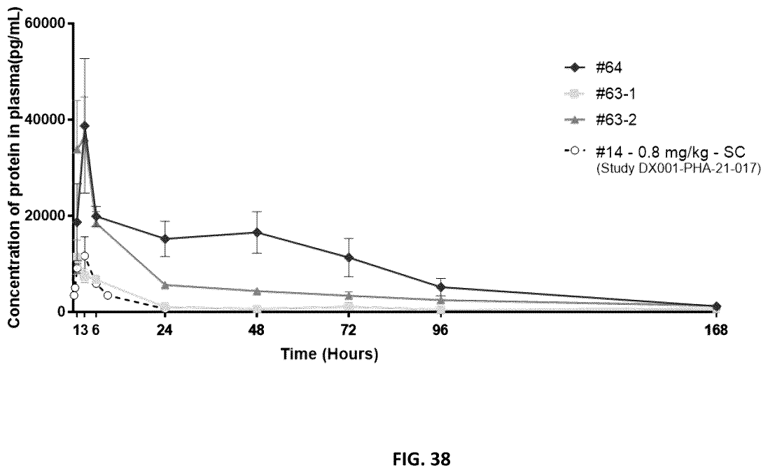

The pharmacokinetic profiles of the Fc-fused variants #64, #63-1 and #63-2 are shown in FIGS. 38 and Table 7. The profiles were overlaid with a single dose profile of the reference protein #14, generated in a previous study (Study No.: DX001-PHA-21-017) for comparison.

| Protein | Tₘₐₓ (h) | Cₘₐₓ (pg/ml) | AUC0-Tₗₐₛₜ |
|---|---|---|---|
| #64 | 3 | 38 713 | 1 622 385 |
| #63-1 | 1 | 11 447 | 229 411 |
| #63-2 | 3 | 36 249 | 825 813 |
| #14 | 3 | 11 663 | 95 828 |

Table 7: Comparison of #64, #63-1 and #63-2 PK parameters. Values for protein #14 generated in a separate study, are shown for comparison (in last row).

The comparison of the 168h-kinetic of the three proteins after subcutaneous administration at 800 μg/kg in 129/Sv mice showed that Cₘₐₓ was reached between 1h and 3h post-dose (Tₘₐₓ).

After this point, the plasma levels of all 3 proteins gradually decreased until the last timepoint of the study (168h). While protein #14 was quantifiable for up to 24h after SC injection, all 3 Fc-fused proteins could still be detected in plasma 168h post-dose, demonstrating the successful extension of the half-life from approximately 6.5h (estimated for #14) to >30h, granted by the Fc-portion (FIG. 38).

6. Production of Rspo1-Fc Fusion Proteins.

The #063 (hRspo1 21-263-Linker-Fc) and #064 (Fc-Linker-hRspo1-21-263) variants were transiently expressed in CHOEBNALT-85-E9 cells. The amount of the two proteins produced is determined. The production yield of the variant #064 is higher than for variant #063 (11.25 mg vs. 4.5 mg).

The #014 (hRspo1 21-263), #064 (Fc-Linker-hRspo1-21-263), #049 (hRspo1 32-263), #121 (Fc-Linker-hRspo1-32-263), variants were transiently expressed in CHOEBNALT-85-E9 cells. The amount of the two proteins produced is determined. The production yield of the variant #064 (16 mg/L) is higher than for variant #014 (9 mg) and the production yield of the variant #121 (20 to 60 mg/L) is higher than for variant #049 (15-20 mg/L).

To determine the influence of linkers on the transient production of variants, the variants as described in Table 8 below were transiently expressed in CHOEBNALT-85-E9 cells and have been tested.

TABLE 8
Key Parameters of selected proteins in Transient pools production.

| | | | | % | | |
|---|---|---|---|---|---|---|
| | | | | Monovalent | | % Low |
| | | Capture | % Full | (2Fc × 1 | | Mol. |
| | | Yields | dimer | Rspo) | % | Weight |
| Variant | Description of the variants: | (mg/50 ml) | Desired | Undesired | Aggregates | species |
| #063 | hRspo1-21-263- | 0.21 | 15.37 | 68.99 | | 15.64 |
| | GGGGGGSGGGGSGGGGSA- | | | | | |
| | Fc | | | | | |
| #190 | hRspo1 21-263- | 0.22 | 28.31 | 58.61 | | 13.08 |
| | GGGGGGSGGGGSA-Fc | | | | | |
| #189 | hRspo1 21-263- | 0.27 | 29.75 | 57.32 | | 12.93 |
| | GGGSGGGGSA-Fc | | | | | |
| #188 | hRspo1 21-263-SGGGGSA-Fc | 0.39 | 31.93 | 54.71 | | 13.36 |
| #155 | hRspo1 32-263-Mutant | 0.81 | 53.55 | 32.67 | 1.66 | 12.12 |
| | H108K; N109D - | | | | | |
| | GGGGGGSGGGGSGGGGSA- | | | | | |
| | Fc | | | | | |
| #194 | hRspo1 32-263-Mutant | 1 | 62.25 | 25.53 | 4.18 | 8.04 |
| | H108K; N109D - | | | | | |
| | GGGGGGSGGGGSA-Fc | | | | | |
| #193 | hRspo1 32-263-Mutant | 1 | 61.78 | 25.17 | 4.14 | 8.91 |
| | H108K; N109D - | | | | | |
| | GGGSGGGGSA-Fc | | | | | |
| #192 | hRspo1 32-263-Mutant | 1.25 | 60.98 | 27.76 | 3.72 | 9.55 |
| | H108K; N109D -SGGGGSA-Fc | | | | | |
| #200 | hRspo1 32-263-Mutant | 0.7 | 71.06 | 26.06 | | 2.88 |
| | H108K; N109D-SA-Fc | | | | | |
| #199 | hRspo1 32-263-SA-Fc | 0.63 | 57.95 | 8.84 + | | 3.96 |
| | | | | 31.25 | | |

The inventors showed that the Fc fusion protein according to the present disclosure with a shorter linker exhibited a better capture and/or % of desired dimers.

The variants were then stable produced by Selexis and the titer and % of fragmentation were analyzed. The results are shown in the Table 9 below:

TABLE 9
Key Parameters of selected proteins in stable pools production

| | Titer | % fragmen- |
|---|---|---|
| Protein | (mg/L) | tation |
| #195 (Fc-hRspo1 32-263) | 900 | 75 |
| #121 (Fc- hRspo1 32-263-Mutant H108K;N109D) | 700 | 82 |
| #049 (hRspo1 32-263 -Mutant H108K;N109D) | 180 | 55 |
| #198 (hRspo1 32-263- GGGGGGSGGGGSA-Fc) | 70 | 38 |
| #199 (hRspo1 32-263 -SA-Fc) | 170 | 52 |
| #192 (hRspo1 32-263 -Mutant H108K;N109D) - | 100 | 35 |
| SGGGGSA-Fc | | |
| #200 (hRspo1 32-263 -Mutant | 120 | 58 |
| H108K;N109D) -SA-Fc | | |

Thus, the fusion of an Fc polypeptide at the N-terminal end of a Variants of the present disclosure allows a high yield of production of the Fc fusion protein.

These data also confirmed that the Fc fusion protein according to the present disclosure with a shorter linker exhibited a higher titer.

7. Optimization of Linker in Fc Fusion Protein

The inventors showed that a linker and Fc optimization is required to generate active Fc fusion protein. Indeed, commercial Fc Fusion protein without linker (Fc (IgG3)-hRspo1; Creative Biomart 053H) was not active in TopFlash assay. Unlike variants #064 or #063 previously tested, Fc-hRspo1 protein (Creative Biomart 053H) does not seem to stimulate pancreatic β-cell proliferation.

Useful Sequences for Practicing the Invention

TABLE 10
Useful amino acid sequences

| SEQ ID | Brief description |
|---|---|
| 1 | Full length Rspo1 aa (with SP) residue 1-263 |
| 2 | Full length Rspo2 aa (with SP) residue 1-243 |
| 3 | Full length Rspo3 aa (with SP) residue 1-272 |
| 4 | Full length Rspo4 aa (with SP) |
| 5 | wt FU1 domain Rspo1 residues 34-85 |
| 6 | wt FU2 domain Rspo1 residues 91-135 |
| 7 | wt TSP domain Rspo1 |
| 8 | wt BR domain Rspo1 |
| 9 | FU1 domain Rspo2 residues 34-85 |
| 10 | FU2 domain Rspo2 residues 91-135 |
| 11 | TSP domain Rspo2 |
| 12 | BR domain Rspo2 |
| 13 | FU1 domain Rspo3 residues 34-85 |
| 14 | FU2 domain Rspo3 residues 91-135 |
| 15 | TSP domain Rspo3 |
| 16 | BR domain Rspo3 residues 208-273 |
| 17 | FU1 domain Rspo4 residues 34-85 |
| 18 | FU2 domain Rspo4 residues 91-135 |
| 19 | TSP domain Rspo4 residues 138-196 |
| 20 | BR domain Rspo4 residues 197-234 |
| 21 | FU1 domain Rspo1 with R66A |
| 22 | Full Length Variant #005 w/o SP (hRspo1 21-263 withN137Q) |
| 23 | Full Length Variant #008 w/o SP (hRspo1 21-263 with R66A) |
| 24 | Full length Variant #009 w/o SP (del aa 21-31 N-term Rspo1) (hRspo1 32-263) |
| 25 | Full length Variant #034 w/o SP (Rspo2 FU1 and Rspo1 otherwise) |
| 26 | Full length Variant #035 w/o SP (Rspo2 FU1 and Rspo1 otherwise) |
| 27 | Full length Variant #047 (hRspo1 32-263with aa substitution E49K V50D D68G D85G) |
| 28 | Full length Variant #051 hRspo1 32-263 with 9 aa substitutions) |
| 29 | Fc fragment to be used in fusion proteins |
| 41 | Full lenght Variant #014 (hRspo1 21-263-(w/o SP)) |
| 42 | Full lenght Variant #063 w/o SP (hRspo1 21-263 (w/o SP)-linker-Fc) |
| 43 | Full length Variant #064 w/o SP (Fc-linker-hRspo1 21-263) |
| 44 | Peptidic linker of variant #063 |
| 45 | Peptidic linker of variant #064 |
| 46 | Peptidic Linker |
| 47 | Full Length Variant #0XX w/o SP (hRspo1 32-263 with aa substitution at position V50R) |
| 48 | Full Length Variant #0XX w/o SP (hRspo1 32-263 with aa substitution at position N51W) |
| 49 | C-terminal aa 144-263 of Rspo1 (as used in most chimeric proteins) |
| 50 | Full Length Variant #026 w/o SP (Chimeric: RSPO1Fu1_32-95-RSPO3Fu2_97-143- |
| | RSPO1_144-263) |
| 51 | Full Length Variant #027 w/o SP (Chimeric: RSPO1Fu1_32-90-RSPO3Fu2_92-143- |
| | RSPO1_144-263) |
| 52 | Full Length Variant #028 w/o SP (Chimeric: RSPO3Fu1_33-96-RSPO1Fu2_96-143- |
| | RSPO1_144-263) |
| 53 | Full Length Variant #029 w/o SP (Chimeric: RSPO3Fu1_33-91-RSPO1Fu2_91-143- |
| | RSPO1_144-263) |
| 54 | Full Length Variant #030 w/o SP (Chimeric. RSPO3Fu1_33-96-RSPO1Fu2_96-143- |
| | RSPO1 144-263 N137Q mutation) |
| 55 | Full Length Variant #031 w/o SP (Chimeric: RSPO2Fu1-Fu2_32-142-RSPO1_144-263) |
| 56 | Full Length Variant #032 w/o SP (Chimeric Fu2-Rspo2 into Rspo1) |
| 57 | Full Length Variant #033 w/o SP (Chimeric: RSPO1Fu1_32-90-RSPO2Fu2_90-142- |
| | RSPO1_144-263) |
| 58 | Full Length Variant #036 w/o SP (Chimeric: RSPO4Fu1-Fu2_27-136-RSPO1_144-263) |
| 59 | Full Length Variant #037 w/o SP (Chimeric Fu2-Rspo4 into Rspo1) |
| 60 | Full Length Variant #038 w/o SP (Chimeric: RSPO1Fu1_32-90-RSPO4Fu2_85-136- |
| | RSPO1_144-263) |
| 61 | Full Length Variant #039 w/o SP (Chimeric: RSPO4Fu1_27-89-RSPO1Fu2 96-143- |
| | RSPO1_144-263) |
| 62 | Full Length Variant #040 w/o SP (Chimeric: RSPO4Fu1_27-84-RSPO1Fu2_91-143- |
| | RSPO1_144-263) |
| 63 | Full Length Variant #042 w/o SP (Chimeric: RSPO2Fu1_32-76-RSPO4Fu2_72-136- |
| | RSPO1_144-263) |
| 64 | Full Length Variant #046 w/o SP (hRSPO1 21-263-short-Linker-Peptide with high affinity |
| | for Albumin) |
| 65 | Full Length Variant #048 w/o SP (hRspo1 32-263 Mutant in Fu1: |
| | E49K; V50D; D68G; D85G; E45L; K55R; N88A) |
| 66 | Full Length Variant #049 w/o SP (hRspo1 32-263 Mutant in Fu2: H108K; N109D) |
| 67 | Full Length Variant #050 w/o SP (hRspo1 32-263 Mutant in Fu1 and Fu2: |
| | E49K; V50D; D68G; D85G; H108K) |
| 68 | Full Length Variant #052 w/o SP (hRspo1 32-263 Mutants in Fu1: E49K; V50D) |
| 69 | Full Length Variant #053 w/o SP (hRspo1 32-263 Mutants in Fu1: D68G; D85G) |
| 70 | Full Length Variant #054 w/o SP (hRspo1 32-263 Mutant in Fu2: H108K) |
| 71 | Full Length Variant #055 w/o SP (hRspo1 32-263 Mutant in Fu1: |
| | L46S; S57Q; I62F; L63F; P77H; F84Y; 195A) |
| 72 | Full Length Variant #056 w/o SP (hRspo1 32-263 Mutant in Fu 2: |
| | E116V; L118F; P127D; A128E; S133F; A136L; G138E; S143V) |
| 73 | Full Length Variant #057 w/o SP (hRspo1 32-263 Mutant in Fu1 and Fu2: |
| | L46S; S57Q; I62F; L63F; P77H; F84Y; I95A; E116V; L118F; |
| | P127D; A128E; S133F; A136L; G138E; S143V)) |
| 74 | Full Length Variant #068 w/o SP (hRspo1 32-263 Mutant in Fu2: H108R) |
| 75 | Full Length Variant #069 w/o SP (hRspo1 32-263 Mutant in Fu2: N109D) |
| 76 | Full Length Variant #071 w/o SP (hRspo1 32-263 Mutant in the BR Domain: L227A) |
| 77 | Full Length Variant #072 w/o SP (hRspo1 32-263 Mutant in the BR Domain: T253A) |
| 78 | Full Length Variant #073 w/o SP (hRspo1 32-263 Mutant in the BR Domain: T253A; |
| | T258A; S259A; S268A if Linker at C-terminal is present |
| 79 | Full Length Variant #074 w/o SP (hRspo1 32-263 Mutant in the BR Domain: G252T) |
| 80 | Full Length Variant #075 w/o SP (hRspo1 32-263 Mutant in the BR Domain: L257S) |
| 81 | Full Length Variant #076 w/o SP (hRspo1 32-263 Mutant in the BR Domain: A260T) |
| 82 | Full Length Variant #077 w/o SP (hRspo1 32-263 Mutant in the BR Domain: A263T) |
| 83 | Full Length Variant #078 w/o SP (hRspo1 32-263 Mutant in the BR Domain: |
| | G252T; L257S; A260T; A263T if Linker at C-terminal is present |
| 84 | Full Length Variant #079 w/o SP (hRspo1 32-263 Mutant in the BR Domain: T253E) |
| 85 | Full Length Variant #080 w/o SP (hRspo1 32-263 Mutant in the BR Domain: T253E, T258E, |
| | S259E, S268E) |
| 86 | Full Length Variant #081 w/o SP (hRspo1 32-263 Mutant in the BR Domain: N253GTN256 |
| | (Native sequence is T253 VGP256) |
| 87 | Full Length Variant #094 w/o SP (Full Length Variant #056 w/o SP (hRSPO1 21-263-Long |
| | Linker-Peptide with high affinity for Albumin |
| 88 | Full Length Variant #095 w/o SP (Peptide with high affinity for Albumin-Linker-hRSPO1 |
| | 21-263 |
| 89 | Full length Variant #082 w/o SP (Rspo1 (32-263) with N-Terminal deletion and R66A |
| | single mutation) |
| 90 | C-Tag and Polyhistidine tag which can be used for purification for example as a C- |
| | terminal tag |
| 91 | Core sequence of peptide binding to serum albumin with high affinity |
| 92 | Full Length Variant #121 (Fc-linker-hRspo1 32-263 with Mutant in Fu2: H108K; N109D) |
| 93 | Full Length Variant #155 (hRspo1 32-263 with Mutant in Fu2: H108K;N109D-linker-Fc ) |
| 94 | Full Length Variant #116 w/o SP (hRspo1 32-244) |
| 95 | Full Length Variant #116 w/o SP (hRspo1 32-244) with Fc fragment at C-Terminal |
| 96 | Full Length Variant #195 w/o SP [(hRspo1 32-263) with Fc fragment at N-Terminal] |
| 97 | Full Length Variant #195 with SP [(hRspo1 32-263) with Fc fragment at N-Terminal] |
| 98 | Full Length Variant #121 w/o SP (hRspo1 32-263 with Mutant in Fu2: H108K; N109D) with |
| | Fc fragment at N-Terminal |
| 99 | Signal peptide |
| 100 | hRspo1 32-263-SA-Fc |
| 101 | hRspo1 32-263 with Mutant in Fu2: H108K; N109D-SA-Fc |
| 102 | Peptidic linker: GGGGGGSGGGGSA |
| 103 | Peptidic linker: GGGSGGGGSA |
| 104 | Peptidic linker: SGGGGSA |
| 105 | hRspo1 32-263-GGGGGGSGGGGSA-Fc |
| 106 | hRspo1 32-263 with Mutant in Fu2: H108K; N109D-GGGGGGSGGGGSA-Fc |
| 107 | hRspo1 32-263 with Mutant in Fu2: H108K; N109D-GGGSGGGGSA-Fc |
| 108 | hRspo1 32-263 with Mutant in Fu2: H108K; N109D-SGGGGSA-Fc |
| 109 | hRspo1 21-263-GGGGGGSGGGGSA-Fc |
| 110 | hRspo1 21-263-GGGSGGGGSA-Fc |
| 111 | hRspo1 21-263-SGGGGSA-Fc |
| 112 | Signal peptide |

TABLE 11
Useful amino acid sequences

| SEQ | |
|---|---|
| ID | Amino acid Sequence |
| 1 | MRLGLCVVALVLSWTHLTISSRGIKGKRQRRISAEGSQACAKGCELCSEVNGCLKCSPKLFILLERNDIRQVGV |
| | CLPSCPPGYFDARNPDMNKCIKCKIEHCEACFSHNFCTKCKEGLYLHKGRCYPACPEGSSAANGTMECSSPA |
| | QCEMSEWSPWGPCSKKQQLCGFRRGSEERTRRVLHAPVGDHAACSDTKETRRCTVRRVPCPEGQKRRKG |
| | GQGRRENANRNLARKESKEAGAGSRRRKGQQQQQQQGTVGPLTSAGPA |
| 2 | MQFRLFSFALIILNCMDYSHCQGNRWRRSKRASYVSNPICKGCLSCSKDNGCSRCQQKLFFFLRREGMRQY |
| | GECLHSCPSGYYGHRAPDMNRCARCRIENCDSCFSKDFCTKCKVGFYLHRGRCFDECPDGFAPLEETMECV |
| | EGCEVGHWSEWGTCSRNNRTCGFKWGLETRTRQIVKKPVKDTILCPTIAESRRCKMTMRHCPGGKRTPKA |
| | KEKRNKKKKRKLIERAQEQHSVFLATDRANQ |
| 3 | MHLRLISWLFIILNFMEYIGSQNASRGRRQRRMHPNVSQGCQGGCATCSDYNGCLSCKPR |
| | LFFALERIGMKQIGVCLSSCPSGYYGTRYPDINKCTKCKADCDTCFNKNFCTKCKSGFYL |
| | HLGKCLDNCPEGLEANNHTMECVSIVHCEVSEWNPWSPCTKKGKTCGFKRGTETRVREII |
| | QHPSAKGNLCPPTNETRKCTVQRKKCQKGERGKKGRERKRKKPNKGESKEAIPDSKSLES |
| | SKEIPEQRENKQQQKKRKVQDKQKSVSVSTVH |
| 4 | MKWVTFISLLFLFSSAYSLNRRKKQVGTGLGGNCTGCIICSEENGCSTCQQRLFLFIRREGIRQYGKCLHD |
| | CPPGYFGIRGQEVNRCKKCGATCESCFSQDFCIRCKRQFYLYKGKCLPTCPPGTLAHQNTRECQGECEL |
| | GPWGGWSPCTHNGKTCGSAWGLESRVREAGRAGHEEAATCQVLSESRKCPIQRPCPGERSPGQKKG |
| | RKDRRPRKDRKLDRRLDVRPRQPGLQP |
| 5 | AEGSQACAKGCELCSEVNGCLKCSPKLFILLERNDIRQVGVCLPSCPPGYFD |
| 6 | MNKCIKCKIEHCEACFSHNFCTKCKEGLYLHKGRCYPA CPEGSSA |
| 7 | SSPAQCEMSEWSPWGPCSKKQQLCGFRRGSEERTRRVLHAPVGDHAACSDTKETRRCTVRRVPCP |
| 8 | EGQKRRKGGQGRRENANRNLARKESKEAGAGSRRRKGQQQQQQQGTVGPLTSAGPA |
| 9 | YVSNPIC KGCLSCSKDN GCSRCQQKLF FFLRREGMRQ YGECLHSCPS GYYGH |
| 10 | NRCARCRIEN CDSCFSKDFC TKCKVGFYLH RGRCFDECPD GFAPL |
| 11 | GCEVGHWSEWGTCSRNNRTCGFKWGLETRTRQIVKKPVKDTILCPTIAESRRCKMTMRHC |
| 12 | PGGKRTPKAKEKRNKKKKRKLIERAQEQHSVFLATDRANQ |
| 13 | HPNVSQG CQGGCATCSD YNGCLSCKPR LFFALERIGM KQIGVCLSSC PSGYY |
| 14 | DINKCTKCKA DCDTCFNKNF CTKCKSGFYL HLGKCLDNCP EGLEA |
| 15 | HCEVSEWNPWSPCTKKGKTCGFKRGTETRVREIIQHPSAKGNLCPPTNETRKCTVQRKKCQ |
| 16 | KGE RGKKGRERKR KKPNKGESKE AIPDSKSLES SKEIPEQREN KQQQKKRKVQ DKQKSVSVST VH |
| 17 | NCTGCII CSEENGCSTC QQRLFLFIRR EGIRQYGKCL HDCPPGYFGI RGQEV |
| 18 | CGATCESCFS QDFCIRCKRQ FYLYKGKCLP TCPPGTLAHQ NTREC |
| 19 | ECELGPWGGWSPCTHNGKTCGSAWGLESRVREAGRAGHEEAATCQVLSESRKCPIQRPC |
| 20 | PGER SPGQKKGRKD RRPRKDRKLD RRLDVRPRQP GLQP |
| 21 | AEGSQACAKGCELCSEVNGCLKCSPKLFILLEANDIRQVGVCLPSCPPGYFD |
| 22 | SRGIKGKRQRRISAEGSQACAKGCELCSEVNGCLKCSPKLFILLERNDIRQVGVCLPSC |
| | PPGYFDARNPDMNKCIKCKIEHCEACFSHNFCTKCKEGLYLHKGRCYPACPEGSSAAQ |
| | GTMECSSPAQCEMSEWSPWGPCSKKQQLCGFRRGSEERTRRVLHAPVGDHAACSDTK |
| | ETRRCTVRRVPCPEGQKRRKGGQGRRENANRNLARKESKEAGAGSRRRKGQQQQQQQ |
| | GTVGPLTSAGPA |
| 23 | SRGIKGKRQRRISAEGSQACAKGCELCSEVNGCLKCSPKLFILLEANDIRQVGVCLPSCPPGYFDARNPDMNK |
| | CIKCKIEHCEACFSHNFCTKCKEGLYLHKGRCYPACPEGSSAANGTMECSSPAQCEMSEWSPWGPCSKKQQL |
| | CGFRRGSEERTRRVLHAPVGDHAACSDTKETRRCTVRRVPCPEGQKRRKGGQGRRENANRNLARKESKEAG |
| | AGSRRRKGQQQQQQQGTVGPLTSAGPA |
| 24 | ISAEGSQACAKGCELCSEVNGCLKCSPKLFILLERNDIRQVGVCLPSCPPGYFDARNPDMNKCIKCKIEHCEACFS |
| | HNFCTKCKEGLYLHKGRCYPACPEGSSAANGTMECSSPAQCEMSEWSPWGPCSKKQQLCGFRRGSEERTRR |
| | VLHAPVGDHAACSDTKETRRCTVRRVPCPEGQKRRKGGQGRRENANRNLARKESKEAGAGSRRRKGQQQQ |
| | QQQGTVGPLTSAGPA |
| 25 | ASYVSNPICKGCLSCSKDNGCSRCQQKLFFFLRREGMRQYGECLHSCPSGYYGHRAPDMNRCAKCKIEHCEA |
| | CFSHNFCTKCKEGLYLHKGRCYPACPEGSSAANGTMECSSPAQCEMSEWSPWGPCSKKQQLCGFRRGSEER |
| | TRRVLHAPVGDHAACSDTKETRRCTVRRVPCPEGQKRRKGGQGRRENANRNLARKESKEAGAGSRRRKGQ |
| | QQQQQQGTVGPLTSAGPA |
| 26 | ASYVSNPICKGCLSCSKDNGCSRCQQKLFFFLRREGMRQYGECLHSCPSGYYGHRAPDMNKCIKCKIEHCEAC |
| | FSHNFCTKCKEGLYLHKGRCYPACPEGSSAANGTMECSSPAQCEMSEWSPWGPCSKKQQLCGFRRGSEERT |
| | RRVLHAPVGDHAACSDTKETRRCTVRRVPCPEGQKRRKGGQGRRENANRNLARKESKEAGAGSRRRKGQQ |
| | QQQQQGTVGPLTSAGPA |
| 27 | ISAEGSQACAKGCELCSKDNGCLKCSPKLFILLERNGIRQ |
| | VGVCLPSCPPGYFGARNPDMNKCIKCKIEHCEACFSHNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 28 | ISAEGSQACAKGCLLCSKDNGCLRCSPKLFILLERNGIRQ |
| | VGVCLPSCPPGYFGARAPDMNKCIKCKIEHCEACFSKDFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 29 | ESKYGPPCPPCPAPEAAGGPSVFLFPPKPKDTLMISRTPEVTCVVVDVSQEDPEVQFNWYVDGVEVHNAKTK |
| | PREEQFNSTYRVVSVLTVLHQDWLNGKEYKCKVSNKGLPSSIEKTISKAKGQPREPQVYTLPPSQEEMTKNQV |
| | SLTCLVKGFYPSDIAVEWESNGQPENNYKTTPPVLDSDGSFFLYSKLTVDKSRWQEGNVFSCSVMHEALHNH |
| | YTQKSLSLSLG |
| 41 | SRGIKGKRQRRISAEGSQACAKGCELCSEVNGCLKCSPKLFILLERNDIRQVGVCLPSCPPGYFDARNPDMNKC |
| | IKCKIEHCEACFSHNFCTKCKEGLYLHKGRCYPACPEGSSAANGTMECSSPAQCEMSEWSPWGPCSKKQQLC |
| | GFRRGSEERTRRVLHAPVGDHAACSDTKETRRCTVRRVPCPEGQKRRKGGQGRRENANRNLARKESKEAGA |
| | GSRRRKGQQQQQQQGTVGPLTSAGPA |
| 42 | SRGIKGKRQRRISAEGSQACAKGCELCSEVNGCLKCSPKLFILLERNDIRQVGVCLPSCPPGYFDARNPDMNKC |
| | IKCKIEHCEACFSHNFCTKCKEGLYLHKGRCYPACPEGSSAANGTMECSSPAQCEMSEWSPWGPCSKKQQLC |
| | GFRRGSEERTRRVLHAPVGDHAACSDTKETRRCTVRRVPCPEGQKRRKGGQGRRENANRNLARKESKEAGA |
| | GSRRRKGQQQQQQQGTVGPLTSAGPAGGGGGGSGGGGSGGGGSAESKYGPPCPPCPAPEAAGGPSVFLF |
| | PPKPKDTLMISRTPEVTCVVVDVSQEDPEVQFNWYVDGVEVHNAKTKPREEQFNSTYRVVSVLTVLHQDWLNG |
| | KEYKCKVSNKGLPSSIEKTISKAKGQPREPQVYTLPPSQEEMTKNQVSLTCLVKGFYPSDIAVEWESNGQPENNYK |
| | TTPPVLDSDGSFFLYSKLTVDKSRWQEGNVFSCSVMHEALHNHYTQKSLSLSLG |
| 43 | ESKYGPPCPPCPAPEAAGGPSVFLFPPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKAKGQPREPQVYTLPPSQEEMTKNQVSL |
| | TCLVKGFYPSDIAVEWESNGQPENNYKTTPPVLDSDGSFFLYSKLTVDKSRWQEGNVFSCSVMHEALHNHYT |
| | QKSLSLSLGGGGGSGGGGSGGGGGGSRGIKGKRQRRISAEGSQACAKGCELCSEVNGCLKCSPKLFILLERNDI |
| | RQVGVCLPSCPPGYFDARNPDMNKCIKCKIEHCEACFSHNFCTKCKEGLYLHKGRCYPACPEGSSAANGTMEC |
| | SSPAQCEMSEWSPWGPCSKKQQLCGFRRGSEERTRRVLHAPVGDHAACSDTKETRRCTVRRVPCPEGQKRRK |
| | GGQGRRENANRNLARKESKEAGAGSRRRKGQQQQQQQGTVGPLTSAGPA |
| 44 | GGGGGGSGGGGSGGGGSA |
| 45 | GGGGSGGGGSGGGGGG |
| 46 | GGGGS |
| 47 | ISAEGSQACAKGCELCSERNGCLKCSPKLFILLERNDIRQVGVCLPSCPPGYFDARNPDMNKCIKCKIEHCEACFSH |
| | NFCTKCKEGLYLHKGRCYPACPEGSSAANGTMECSSPAQCEMSEWSPWGPCSKKQQLCGFRRGSEERTRRVLH |
| | APVGDHAACSDTKETRRCTVRRVPCPEGQKRRKGGQGRRENANRNLARKESKEAGAGSRRRKGQQQQQQQG |
| | TVGPLTSAGPA |
| 48 | ISAEGSQACAKGCELCSEVWGCLKCSPKLFILLERNDIRQVGVCLPSCPPGYFDARNPDMNKCIKCKIEHCEACFSH |
| | NFCTKCKEGLYLHKGRCYPACPEGSSAANGTMECSSPAQCEMSEWSPWGPCSKKQQLCGFRRGSEERTRRVLH |
| | APVGDHAACSDTKETRRCTVRRVPCPEGQKRRKGGQGRRENANRNLARKESKEAGAGSRRRKGQQQQQQQG |
| | TVGPLTSAGPA |
| 49 | SPAQCEMSEWSPWGPCSKKQQLCGFRRGSEERTRRVLHAPVGDHAACSDTKETRRCTVRRVPCPEGQKRRKG |
| | GQGRRENANRNLARKESKEAGAGSRRRKGQQQQQQQGTVGPLTSAGPA |
| 50 | ISAEGSQACAKGCELCSEVNGCLKCSPKLFILLERNDIRQVGVCLPSCPPGYFDARNPDMNKCIKCKADCDTCFNK |
| | NFCTKCKSGFYLHLGKCLDNCPEGLEANNHTMECVSPAQCEMSEWSPWGPCSKKQQLCGFRRGSEERTRRVL |
| | HAPVGDHAACSDTKETRRCTVRRVPCPEGQKRRKGGQGRRENANRNLARKESKEAGAGSRRRKGQQQQQQ |
| | QGTVGPLTSAGPA |
| 51 | ISAEGSQACAKGCELCSEVNGCLKCSPKLFILLERNDIRQVGVCLPSCPPGYFDARNPDINKCTKCKADCDTCFN |
| | KNFCTKCKSGFYLHLGKCLDNCPEGLEANNHTMECVSPAQCEMSEWSPWGPCSKKQQLCGFRRGSEERTRR |
| | VLHAPVGDHAACSDTKETRRCTVRRVPCPEGQKRRKGGQGRRENANRNLARKESKEAGAGSRRRKGQQQQ |
| | QQQGTVGPLTSAGPA |
| 52 | MHPNVSQGCQGGCATCSDYNGCLSCKPRLFFALERIGMKQIGVCLSSCPSGYYGTRYPDINKCTKCKIEHCEA |
| | CFSHNFCTKCKEGLYLHKGRCYPACPEGSSAANGTMECSSPAQCEMSEWSPWGPCSKKQQLCGFRRGSEER |
| | TRRVLHAPVGDHAACSDTKETRRCTVRRVPCPEGQKRRKGGQGRRENANRNLARKESKEAGAGSRRRKGQ |
| | QQQQQQGTVGPLTSAGPA |
| 53 | MHPNVSQGCQGGCATCSDYNGCLSCKPRLFFALERIGMKQIGVCLSSCPSGYYGTRYPDMNKCIKCKIEHCE |
| | ACFSHNFCTKCKEGLYLHKGRCYPACPEGSSAANGTMECSSPAQCEMSEWSPWGPCSKKQQLCGFRRGS |
| | EERTRRVLHAPVGDHAACSDTKETRRCTVRRVPCPEGQKRRKGGQGRRENANRNLARKESKEAGAGSRRRK |
| | GQQQQQQQGTVGPLTSAGPA |
| 54 | MHPNVSQGCQGGCATCSDYNGCLSCKPRLFFALERIGMKQIGVCLSSCPSGYYGTRYPDINKCTKCKIEHCEA |
| | CFSHNFCTKCKEGLYLHKGRCYPACPEGSSAAQGTMECSSPAQCEMSEWSPWGPCSKKQQLCGFRRGSEER |
| | TRRVLHAPVGDHAACSDTKETRRCTVRRVPCPEGQKRRKGGQGRRENANRNLARKESKEAGAGSRRRKGQ |
| | QQQQQQGTVGPLTSAGPA |
| 55 | ASYVSNPICKGCLSCSKDNGCSRCQQKLFFFLRREGMRQYGECLHSCPSGYYGHRAPDMNRCARCRIENCD |
| | SCFSKDFCTKCKVGFYLHRGRCFDECPDGFAPLEETMECVSPAQCEMSEWSPWGPCSKKQQLCGFRRGSE |
| | ERTRRVLHAPVGDHAACSDTKETRRCTVRRVPCPEGQKRRKGGQGRRENANRNLARKESKEAGAGSRRRK |
| | GQQQQQQQGTVGPLTSAGPA |
| 56 | ISAEGSQACAKGCELCSEVNGCLKCSPKLFILLERNDIRQVGVCLPSCPPGYFDARNPDMNKCIRCRIENCDS |
| | CFSKDFCTKCKVGFYLHRGRCFDECPDGFAPLEETMECVSPAQCEMSEWSPWGPCSKKQQLCGFRRGSEE |
| | RTRRVLHAPVGDHAACSDTKETRRCTVRRVPCPEGQKRRKGGQGRRENANRNLARKESKEAGAGSRRRK |
| | GQQQQQQQGTVGPLTSAGPA |
| 57 | ISAEGSQACAKGCELCSEVNGCLKCSPKLFILLERNDIRQVGVCLPSCPPGYFDARNPDMNRCARCRIENCD |
| | SCFSKDFCTKCKVGFYLHRGRCFDECPDGFAPLEETMECVSPAQCEMSEWSPWGPCSKKQQLCGFRRGS |
| | EERTRRVLHAPVGDHAACSDTKETRRCTVRRVPCPEGQKRRKGGQGRRENANRNLARKESKEAGAGSRR |
| | RKGQQQQQQQGTVGPLTSAGPA |
| 58 | LNVGTGLGGNCTGCIICSEENGCSTCQQRLFLFIRREGIRQYGKCLHDCPPGYFGIRGQEVNRCKKCGATCE |
| | SCFSQDFCIRCKRQFYLYKGKCLPTCPPGTLAHQNTRECQSPAQCEMSEWSPWGPCSKKQQLCGFRRGS |
| | EERTRRVLHAPVGDHAACSDTKETRRCTVRRVPCPEGQKRRKGGQGRRENANRNLARKESKEAGAGSRR |
| | RKGQQQQQQQGTVGPLTSAGPA |
| 59 | ISAEGSQACAKGCELCSEVNGCLKCSPKLFILLERNDIRQVGVCLPSCPPGYFDARNPDMNKCIKCGATCESC |
| | FSQDFCIRCKRQFYLYKGKCLPTCPPGTLAHQNTRECQSPAQCEMSEWSPWGPCSKKQQLCGFRRGSEERT |
| | RRVLHAPVGDHAACSDTKETRRCTVRRVPCPEGQKRRKGGQGRRENANRNLARKESKEAGAGSRRRKGQQ |
| | QQQQQGTVGPLTSAGPA |
| 60 | ISAEGSQACAKGCELCSEVNGCLKCSPKLFILLERNDIRQVGVCLPSCPPGYFDARNPDVNRCKKCGATCESC |
| | FSQDFCIRCKRQFYLYKGKCLPTCPPGTLAHQNTRECQSPAQCEMSEWSPWGPCSKKQQLCGFRRGSEER |
| | TRRVLHAPVGDHAACSDTKETRRCTVRRVPCPEGQKRRKGGQGRRENANRNLARKESKEAGAGSRRRKG |
| | QQQQQQQGTVGPLTSAGPA |
| 61 | LNVGTGLGGNCTGCIICSEENGCSTCQQRLFLFIRREGIRQYGKCLHDCPPGYFGIRGQEVNRCKKCKIEHCE |
| | ACFSHNFCTKCKEGLYLHKGRCYPACPEGSSAANGTMECSSPAQCEMSEWSPWGPCSKKQQLCGFRRGSEE |
| | RTRRVLHAPVGDHAACSDTKETRRCTVRRVPCPEGQKRRKGGQGRRENANRNLARKESKEAGAGSRRRKGQ |
| | QQQQQQGTVGPLTSAGPA |
| 62 | LNVGTGLGGNCTGCIICSEENGCSTCQQRLFLFIRREGIRQYGKCLHDCPPGYFGIRGQEMNKCIKCKIEHCEAC |
| | FSHNFCTKCKEGLYLHKGRCYPACPEGSSAANGTMECSSPAQCEMSEWSPWGPCSKKQQLCGFRRGSEERTR |
| | RVLHAPVGDHAACSDTKETRRCTVRRVPCPEGQKRRKGGQGRRENANRNLARKESKEAGAGSRRRKGQQQQ |
| | QQQGTVGPLTSAGPA |
| 63 | ASYVSNPICKGCLSCSKDNGCSRCQQKLFFFLRREGMRQYGECLHDCPPGYFGIRGQEVNRCKKCGATCESC |
| | FSQDFCIRCKRQFYLYKGKCLPTCPPGTLAHQNTRECQSPAQCEMSEWSPWGPCSKKQQLCGFRRGSEERT |
| | RRVLHAPVGDHAACSDTKETRRCTVRRVPCPEGQKRRKGGQGRRENANRNLARKESKEAGAGSRRRKGQQQQ |
| | QQQGTVGPLTSAGPA |
| 64 | SRGIKGKRQRRISAEGSQACAKGCELCSEVNGCLKCSPKLFILLERNDIRQVGVCLPSCPPGYFDARNPDMN |
| | KCIKCKIEHCEACFSHNFCTKCKEGLYLHKGRCYPACPEGSSAANGTMECSSPAQCEMSEWSPWGPCSKKQ |
| | QLCGFRRGSEERTRRVLHAPVGDHAACSDTKETRRCTVRRVPCPEGQKRRKGGQGRRENANRNLARKESK |
| | EAGAGSRRRKGQQQQQQQGTVGPLTSAGPAGGGGSQRLMEDICLPRWGCLWEDDF |
| 65 | ISAEGSQACAKGCLLCSKDNGCLRCSPKLFILLERNGIRQ |
| | VGVCLPSCPPGYFGARAPDMNKCIKCKIEHCEACFSHNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 66 | ISAEGSQACAKGCELCSEVNGCLKCSPKLFILLERNDIRQ |
| | VGVCLPSCPPGYFDARNPDMNKCIKCKIEHCEACFSKDFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 67 | ISAEGSQACAKGCELCSKDNGCLKCSPKLFILLERNGIRQ |
| | VGVCLPSCPPGYFGARNPDMNKCIKCKIEHCEACFSKNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 68 | ISAEGSQACAKGCELCSKDNGCLKCSPKLFILLERNDIRQ |
| | VGVCLPSCPPGYFDARNPDMNKCIKCKIEHCEACFSHNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 69 | ISAEGSQACAKGCELCSEVNGCLKCSPKLFILLERNGIRQ |
| | VGVCLPSCPPGYFGARNPDMNKCIKCKIEHCEACFSHNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 70 | ISAEGSQACAKGCELCSEVNGCLKCSPKLFILLERNDIRQ |
| | VGVCLPSCPPGYFDARNPDMNKCIKCKIEHCEACFSKNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 71 | ISAEGSQACAKGCESCSEVNGCLKCQPKLFFFLERNDIRQ |
| | VGVCLHSCPPGYYDARNPDMNKCAKCKIEHCEACFSHNFCTKCKEGLYLHKGRCYPACPE |
| | GSSAANGTMECSSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 72 | ISAEGSQACAKGCELCSEVNGCLKCSPKLFILLERNDIRQ |
| | VGVCLPSCPPGYFDARNPDMNKCIKCKIEHCEACFSHNFCTKCKVGFYLHKGRCYDECPE |
| | GFSALNETMECVSPAQCEMSEWSPWGPCSKKQQLCGFRRGSEERTRRVLHAPVGDHAACS |
| | DTKETRRCTVRRVPCPEGQKRRKGGQGRRENANRNLARKESKEAGAGSRRRKGQQQQQQQ |
| | GTVGPLTSAGPA |
| 73 | ISAEGSQACAKGCESCSEVNGCLKCQPKLFFFLERNDIRQVGVCLHSCPPGYYDARN |
| | PDMNKCAKCKIEHCEACFSHNFCTKCKVGFYLHKGRCYDECPEGFSALNETMECVSP |
| | AQCEMSEWSPWGPCSKKQQLCGFRRGSEERTRRVLHAPVGDHAACSDTKETRRCT |
| | VRRVPCPEGQKRRKGGQGRRENANRNLARKESKEAGAGSRRRKGQQQQQQQGTV |
| | GPLTSAGPA |
| 74 | ISAEGSQACAKGCELCSEVNGCLKCSPKLFILLERNDIRQVGVCLPSCPPGYFDARN |
| | PDMNKCIKCKIEHCEACFSRNFCTKCKEGLYLHKGRCYPACPEGSSAANGTMECSS |
| | PAQCEMSEWSPWGPCSKKQQLCGFRRGSEERTRRVLHAPVGDHAACSDTKETRR |
| | CTVRRVPCPEGQKRRKGGQGRRENANRNLARKESKEAGAGSRRRKGQQQQQQQ |
| | GTVGPLTSAGPA |
| 75 | ISAEGSQACAKGCELCSEVNGCLKCSPKLFILLERNDIRQVGVCLPSCPPGYFDARNP |
| | DMNKCIKCKIEHCEACFSHDFCTKCKEGLYLHKGRCYPACPEGSSAANGTMECSSPA |
| | QCEMSEWSPWGPCSKKQQLCGFRRGSEERTRRVLHAPVGDHAACSDTKETRRCTV |
| | RRVPCPEGQKRRKGGQGRRENANRNLARKESKEAGAGSRRRKGQQQQQQQGTVG |
| | PLTSAGPA |
| 76 | ISAEGSQACAKGCELCSEVNGCLKCSPKLFILLERNDIRQVGVCLPSCPPGYFDARNP |
| | DMNKCIKCKIEHCEACFSHNFCTKCKEGLYLHKGRCYPACPEGSSAANGTMECSSPA |
| | QCEMSEWSPWGPCSKKQQLCGFRRGSEERTRRVLHAPVGDHAACSDTKETRRCTV |
| | RRVPCPEGQKRRKGGQGRRENANRNAARKESKEAGAGSRRRKGQQQQQQQGTVG |
| | PLTSAGPA |
| 77 | ISAEGSQACAKGCELCSEVNGCLKCSPKLFILLERNDIRQVGVCLPSCPPGYFDARNPD |
| | MNKCIKCKIEHCEACFSHNFCTKCKEGLYLHKGRCYPACPEGSSAANGTMECSSPAQC |
| | EMSEWSPWGPCSKKQQLCGFRRGSEERTRRVLHAPVGDHAACSDTKETRRCTVRRV |
| | PCPEGQKRRKGGQGRRENANRNLARKESKEAGAGSRRRKGQQQQQQQGAVGPLTS |
| | AGPA |
| 78 | ISAEGSQACAKGCELCSEVNGCLKCSPKLFILLERNDIRQVGVCLPSCPPGYFDARNPD |
| | MNKCIKCKIEHCEACFSHNFCTKCKEGLYLHKGRCYPACPEGSSAANGTMECSSPAQC |
| | EMSEWSPWGPCSKKQQLCGFRRGSEERTRRVLHAPVGDHAACSDTKETRRCTVRRV |
| | PCPEGQKRRKGGQGRRENANRNLARKESKEAGAGSRRRKGQQQQQQQGAVGPLAA |
| | AGPAGGGGAEPEAHHHHHH |
| 79 | ISAEGSQACAKGCELCSEVNGCLKCSPKLFILLERNDIRQVGVCLPSCPPGYFDARNPDM |
| | NKCIKCKIEHCEACFSHNFCTKCKEGLYLHKGRCYPACPEGSSAANGTMECSSPAQCEM |
| | SEWSPWGPCSKKQQLCGFRRGSEERTRRVLHAPVGDHAACSDTKETRRCTVRRVPCP |
| | EGQKRRKGGQGRRENANRNLARKESKEAGAGSRRRKGQQQQQQQTTVGPLTSAGPA |
| 80 | ISAEGSQACAKGCELCSEVNGCLKCSPKLFILLERNDIRQVGVCLPSCPPGYFDARNPDM |
| | NKCIKCKIEHCEACFSHNFCTKCKEGLYLHKGRCYPACPEGSSAANGTMECSSPAQCEM |
| | SEWSPWGPCSKKQQLCGFRRGSEERTRRVLHAPVGDHAACSDTKETRRCTVRRVPCPE |
| | GQKRRKGGQGRRENANRNLARKESKEAGAGSRRRKGQQQQQQQGTVGPSTSAGPA |
| 81 | ISAEGSQACAKGCELCSEVNGCLKCSPKLFILLERNDIRQVGVCLPSCPPGYFDARNPDMN |
| | KCIKCKIEHCEACFSHNFCTKCKEGLYLHKGRCYPACPEGSSAANGTMECSSPAQCEMSE |
| | WSPWGPCSKKQQLCGFRRGSEERTRRVLHAPVGDHAACSDTKETRRCTVRRVPCPEGQ |
| | KRRKGGQGRRENANRNLARKESKEAGAGSRRRKGQQQQQQQGTVGPLTSTGPA |
| 82 | ISAEGSQACAKGCELCSEVNGCLKCSPKLFILLERNDIRQVGVCLPSCPPGYFDARNPDM |
| | NKCIKCKIEHCEACFSHNFCTKCKEGLYLHKGRCYPACPEGSSAANGTMECSSPAQCEM |
| | SEWSPWGPCSKKQQLCGFRRGSEERTRRVLHAPVGDHAACSDTKETRRCTVRRVPCPE |
| | GQKRRKGGQGRRENANRNLARKESKEAGAGSRRRKGQQQQQQQGTVGPLTSAGPT |
| 83 | ISAEGSQACAKGCELCSEVNGCLKCSPKLFILLERNDIRQVGVCLPSCPPGYFDARNPDM |
| | NKCIKCKIEHCEACFSHNFCTKCKEGLYLHKGRCYPACPEGSSAANGTMECSSPAQCEM |
| | SEWSPWGPCSKKQQLCGFRRGSEERTRRVLHAPVGDHAACSDTKETRRCTVRRVPCPE |
| | GQKRRKGGQGRRENANRNLARKESKEAGAGSRRRKGQQQQQQQTTVGPSTSTGPT |
| 84 | ISAEGSQACAKGCELCSEVNGCLKCSPKLFILLERNDIRQVGVCLPSCPPGYFDARNPDMN |
| | KCIKCKIEHCEACFSHNFCTKCKEGLYLHKGRCYPACPEGSSAANGTMECSSPAQCEMSE |
| | WSPWGPCSKKQQLCGFRRGSEERTRRVLHAPVGDHAACSDTKETRRCTVRRVPCPEGQ |
| | KRRKGGQGRRENANRNLARKESKEAGAGSRRRKGQQQQQQQGEVGPLTSAGPA |
| 85 | ISAEGSQACAKGCELCSEVNGCLKCSPKLFILLERNDIRQVGVCLPSCPPGYFDARNPDMN |
| | KCIKCKIEHCEACFSHNFCTKCKEGLYLHKGRCYPACPEGSSAANGTMECSSPAQCEMSE |
| | WSPWGPCSKKQQLCGFRRGSEERTRRVLHAPVGDHAACSDTKETRRCTVRRVPCPEGQ |
| | KRRKGGQGRRENANRNLARKESKEAGAGSRRRKGQQQQQQQGEVGPLEEAGPAGGGG |
| | EEPEAHHHHHH |
| 86 | ISAEGSQACAKGCELCSEVNGCLKCSPKLFILLERNDIRQVGVCLPSCPPGYFDARNPDM |
| | NKCIKCKIEHCEACFSHNFCTKCKEGLYLHKGRCYPACPEGSSAANGTMECSSPAQCEM |
| | SEWSPWGPCSKKQQLCGFRRGSEERTRRVLHAPVGDHAACSDTKETRRCTVRRVPCP |
| | EGQKRRKGGQGRRENANRNLARKESKEAGAGSRRRKGQQQQQQQGNGTNLTSAGPA |
| 87 | SRGIKGKRQRRISAEGSQACAKGCELCSEVNGCLKCSPKLFILLERNDIRQVGVCLPSCPP |
| | GYFDARNPDMNKCIKCKIEHCEACFSHNFCTKCKEGLYLHKGRCYPACPEGSSAANGTM |
| | ECSSPAQCEMSEWSPWGPCSKKQQLCGFRRGSEERTRRVLHAPVGDHAACSDTKETR |
| | RCTVRRVPCPEGQKRRKGGQGRRENANRNLARKESKEAGAGSRRRKGQQQQQQQGT |
| | VGPLTSAGPAGGGGSGGGGSGGGGGGQRLMEDICLPRWGCLWEDDF |
| 88 | QRLMEDICLPRWGCLWEDDFGGGGSGGGGSGGGGGGSRGIKGKRQRRISAEGSQACA |
| | KGCELCSEVNGCLKCSPKLFILLERNDIRQVGVCLPSCPPGYFDARNPDMNKCIKCKIEHC |
| | EACFSHNFCTKCKEGLYLHKGRCYPACPEGSSAANGTMECSSPAQCEMSEWSPWGPCS |
| | KKQQLCGFRRGSEERTRRVLHAPVGDHAACSDTKETRRCTVRRVPCPEGQKRRKGGQG |
| | RRENANRNLARKESKEAGAGSRRRKGQQQQQQQGTVGPLTSAGP |
| 89 | ISAEGSQACAKGCELCSEVNGCLKCSPKLFILLEANDIRQVGVCLPSCPPGYFDARNPDMNKCIKCKIEHCEACFSHNFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKGQQQQQQQGTVGPLTSAGPA |
| 90 | GGGGSEPEAHHHHHH |
| 91 | DICLPRWGCLW |
| 92 | MALWMRFLPLLALLVLWEPKPAQAESKYGPPCPPCPAPEAAGGPSVFLFPPKPKDTLMISRTPEVTC |
| | VVVDVSQEDPEVQFNWYVDGVEVHNAKTKPREEQFNSTYRVVSVLTVLHQDWLNGKEYKCKVSNK |
| | GLPSSIEKTISKAKGQPREPQVYTLPPSQEEMTKNQVSLTCLVKGFYPSDIAVEWESNGQPENNYKTTP |
| | PVLDSDGSFFLYSKLTVDKSRWQEGNVFSCSVMHEALHNHYTQKSLSLSLGGGGGSGGGGSGGGGG |
| | GISAEGSQACAKGCELCSEVNGCLKCSPKLFILLERNDIRQVGVCLPSCPPGYFDARNPDMNKCIKCKIEH |
| | CEACFSKDFCTKCKEGLYLHKGRCYPACPEGSSAANGTMECSSPAQCEMSEWSPWGPCSKKQQLCGF |
| | RRGSEERTRRVLHAPVGDHAACSDTKETRRCTVRRVPCPEGQKRRKGGQGRRENANRNLARKESKEAG |
| | AGSRRRKGQQQQQQQGTVGPLTSAGPA |
| 93 | ISAEGSQACAKGCELCSEVNGCLKCSPKLFILLERNDIRQVGVCLPSCPPGYFDARN |
| | PDMNKCIKCKIEHCEACFSKDFCTKCKEGLYLHKGRCYPACPEGSSAANGTMECSSPAQCEMSEWSPWGPCSKK |
| | QQLCGFRRGSEERTRRVLHAPVGDHAACSDTKETRRCTVRRVPCPEGQKRRKGGQGRRENANRNLARKESKEA |
| | GAGSRRRKGQQQQQQQGTVGPLTSAGPAGGGGGGSGGGGSGGGGSAESKYGPPCPPCPAPEAAGGPSVFLF |
| | PPKPKDTLMISRTPEVTCVVVDVSQEDPEVQFNWYVDGVEVHNAKTKPREEQFNSTYRVVSVLTVLHQDWLNGK |
| | EYKCKVSNKGLPSSIEKTISKAKGQPREPQVYTLPPSQEEMTKNQVSLTCLVKGFYPSDIAVEWESNGQPENNYKTT |
| | PPVLDSDGSFFLYSKLTVDKSRWQEGNVFSCSVMHEALHNHYTQKSLSLSLG |
| 94 | ISAEGSQACAKGCELCSEVNGCLKCSPKLFILLERNDIRQVGVCLPSCPPGYFDARNPDMNKCIKCKIEHCEACFSHNFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKG |
| 95 | ISAEGSQACAKGCELCSEVNGCLKCSPKLFILLERNDIRQVGVCLPSCPPGYFDARNPDMNK |
| | CIKCKIEHCEACFSHNFCTKCKEGLYLHKGRCYPACPEGSSAANGTMECSSPAQCEMSEWSPWGPCSKKQQLCGFRRGSE |
| | ERTRRVLHAPVGDHAACSDTKETRRCTVRRVPCPEGQKRRKGGQGRRENANRNLARKESKEAGAGSRRRKGGGGGGGS |
| | GGGGSGGGGSAESKYGPPCPPCPAPEAAGGPSVFLFPPKPKDTLMISRTPEVTCVVVDVSQEDPEVQFNWYVDGVEVHN |
| | AKTKPREEQFNSTYRVVSVLTVLHQDWLNGKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSKLTVDKSRWQEGNVFSCSVMHEALHNHYTQKSLSLSLG |
| 96 | ESKYGPPCPPCPAPEAAGGPSVFLFPPKPKDTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEMTKNQVSLTCLVKGFYPSDIAVE |
| | WESNGQPENNYKTTPPVLDSDGSFFLYSKLTVDKSRWQEGNVFSCSVMHEALHNHYTQKSLSLSLGGGGGSGGGGSGG |
| | GGGGISAEGSQACAKGCELCSEVNGCLKCSPKLFILLERNDIRQVGVCLPSCPPGYFDARNPDMNKCIKCKIEHCEACFSHN |
| | FCTKCKEGLYLHKGRCYPACPEGSSAANGTMECSSPAQCEMSEWSPWGPCSKKQQLCGFRRGSEERTRRVLHAPVGDH |
| | AACSDTKETRRCTVRRVPCPEGQKRRKGGQGRRENANRNLARKESKEAGAGSRRRKGQQQQQQQGTVGPLTSAGPA |
| 97 | MALWMRFLPLLALLVLWEPKPAQAESKYGPPCPPCPAPEAAGGPSVFLFPPKPKDTLMISRTPEVTCVVVDVSQEDPEVQ |
| | FNWYVDGVEVHNAKTKPREEQFNSTYRVVSVLTVLHQDWLNGKEYKCKVSNKGLPSSIEKTISKAKGQPREPQVYTLPPS |
| | QEEMTKNQVSLTCLVKGFYPSDIAVEWESNGQPENNYKTTPPVLDSDGSFFLYSKLTVDKSRWQEGNVFSCSVMHEALH |
| | NHYTQKSLSLSLGGGGGSGGGGSGGGGGGISAEGSQACAKGCELCSEVNGCLKCSPKLFILLERNDIRQVGVCLPSCPPGY |
| | FDARNPDMNKCIKCKIEHCEACFSHNFCTKCKEGLYLHKGRCYPACPEGSSAANGTMECSSPAQCEMSEWSPWGPCSKK |
| | QQLCGFRRGSEERTRRVLHAPVGDHAACSDTKETRRCTVRRVPCPEGQKRRKGGQGRRENANRNLARKESKEAGAGSRR |
| | RKGQQQQQQQGTVGPLTSAGPA |
| 98 | ESKYGPPCPPCPAPEAAGGPSVFLFPPKPKDTLMISRTPEVTCVVVDVSQEDPEVQFNWYVDGVEVHNAKTKPREEQFNS |
| | TYRVVSVLTVLHQDWLNGKEYKCKVSNKGLPSSIEKTISKAKGQPREPQVYTLPPSQEEMTKNQVSLTCLVKGFYPSDIAVE |
| | WESNGQPENNYKTTPPVLDSDGSFFLYSKLTVDKSRWQEGNVFSCSVMHEALHNHYTQKSLSLSLGGGGGSGGGGSGG |
| | GGGGISAEGSQACAKGCELCSEVNGCLKCSPKLFILLERNDIRQVGVCLPSCPPGYFDARNPDMNKCIKCKIEHCEACFSKD |
| | FCTKCKEGLYLHKGRCYPACPEGSSAANGTMECSSPAQCEMSEWSPWGPCSKKQQLCGFRRGSEERTRRVLHAPVGDH |
| | AACSDTKETRRCTVRRVPCPEGQKRRKGGQGRRENANRNLARKESKEAGAGSRRRKGQQQQQQQGTVGPLTSAGPA |
| 99 | MALWMRFLPLLALLVLWEPKPAQA |
| 100 | ISAEGSQACAKGCELCSEVNGCLKCSPKLFILLERNDIRQVGVCLPSCPPGYFDARNPDMNKCIKCKIEHCEACFS |
| | HNFCTKCKEGLYLHKGRCYPACPEGSSAANGTMECSSPAQCEMSEWSPWGPCSKKQQLCGFRRGSEERTRR |
| | VLHAPVGDHAACSDTKETRRCTVRRVPCPEGQKRRKGGQGRRENANRNLARKESKEAGAGSRRRKGQQQQ |
| | QQQGTVGPLTSAGPASAESKYGPPCPPCPAPEAAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSKLTVDKSRWQEGNVFSCSVMHEALHNHYTQKSL |
| | SLSLG |
| 101 | ISAEGSQACAKGCELCSEVNGCLKCSPKLFILLERNDIRQVGVCLPSCPPGYFDARNPDMNKCIKCKIEHCEACFSKDFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKGQQQQQQQGTVGPLTSAGPASAESKYG |
| | PPCPPCPAPEAAGGPSVFLFPPKPKDTLMISRTPEVTCVVVDVSQEDPEVQFNWYVDGVEVHNAKTKPREEQFNSTYRVV |
| | SVLTVLHQDWLNGKEYKCKVSNKGLPSSIEKTISKAKGQPREPQVYTLPPSQEEMTKNQVSLTCLVKGFYPSDIAVEWESN |
| | GQPENNYKTTPPVLDSDGSFFLYSKLTVDKSRWQEGNVFSCSVMHEALHNHYTQKSLSLSLG |
| 102 | GGGGGGSGGGGSA |
| 103 | GGGSGGGGSA |
| 104 | SGGGGSA |
| 105 | ISAEGSQACAKGCELCSEVNGCLKCSPKLFILLERNDIRQVGVCLPSCPPGYFDARNPDMNKCIKCKIEHCEACFSHNFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKGQQQQQQQGTVGPLTSAGPAGGGGG |
| | GSGGGGSAESKYGPPCPPCPAPEAAGGPSVFLFPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSKLTVDKSRWQEGNVFSCSVMHEALHNHYTQKSLSLSLG |
| 106 | ISAEGSQACAKGCELCSEVNGCLKCSPKLFILLERNDIRQVGVCLPSCPPGYFDARNPDMNKCIKCKIEHCEACFSKDFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKGQQQQQQQGTVGPLTSAGPASAGGGG |
| | GGSGGGGSAESKYGPPCPPCPAPEAAGGPSVFLFPPKPKDTLMISRTPEVTCVVVDVSQEDPEVQFNWYVDGVEVHNAK |
| | TKPREEQFNSTYRVVSVLTVLHQDWLNGKEYKCKVSNKGLPSSIEKTISKAKGQPREPQVYTLPPSQEEMTKNQVSLTCLVK |
| | GFYPSDIAVEWESNGQPENNYKTTPPVLDSDGSFFLYSKLTVDKSRWQEGNVFSCSVMHEALHNHYTQKSLSLSLG |
| 107 | ISAEGSQACAKGCELCSEVNGCLKCSPKLFILLERNDIRQVGVCLPSCPPGYFDARNPDMNKCIKCKIEHCEACFSKDFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKGQQQQQQQGTVGPLTSAGPASAGGGS |
| | GGGGSAESKYGPPCPPCPAPEAAGGPSVFLFPPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKAKGQPREPQVYTLPPSQEEMTKNQVSLTCLVKGFY |
| | PSDIAVEWESNGQPENNYKTTPPVLDSDGSFFLYSKLTVDKSRWQEGNVFSCSVMHEALHNHYTQKSLSLSLG |
| 108 | ISAEGSQACAKGCELCSEVNGCLKCSPKLFILLERNDIRQVGVCLPSCPPGYFDARNPDMNKCIKCKIEHCEACFSKDFCTKC |
| | KEGLYLHKGRCYPACPEGSSAANGTMECSSPAQCEMSEWSPWGPCSKKQQLCGFRRGSEERTRRVLHAPVGDHAACSD |
| | TKETRRCTVRRVPCPEGQKRRKGGQGRRENANRNLARKESKEAGAGSRRRKGQQQQQQQGTVGPLTSAGPASASGGG |
| | SAESKYGPPCPPCPAPEAAGGPSVFLFPPKPKDTLMISRTPEVTCVVVDVSQEDPEVQFNWYVDGVEVHNAKTKPREEQF |
| | NSTYRVVSVLTVLHQDWLNGKEYKCKVSNKGLPSSIEKTISKAKGQPREPQVYTLPPSQEEMTKNQVSLTCLVKGFYPSDIA |
| | VEWESNGQPENNYKTTPPVLDSDGSFFLYSKLTVDKSRWQEGNVFSCSVMHEALHNHYTQKSLSLSLG |
| 109 | SRGIKGKRQRRISAEGSQACAKGCELCSEVNGCLKCSPKLFILLERNDIRQVGVCLPSCPPGYFDARNPDMNKCIKCKIEHCE |
| | ACFSHNFCTKCKEGLYLHKGRCYPACPEGSSAANGTMECSSPAQCEMSEWSPWGPCSKKQQLCGFRRGSEERTRRVLHA |
| | PVGDHAACSDTKETRRCTVRRVPCPEGQKRRKGGQGRRENANRNLARKESKEAGAGSRRRKGQQQQQQQGTVGPLTS |
| | AGPAGGGGGGSGGGGSAESKYGPPCPPCPAPEAAGGPSVFLFPPKPKDTLMISRTPEVTCVVVDVSQEDPEVQFNWYVD |
| | GVEVHNAKTKPREEQFNSTYRVVSVLTVLHQDWLNGKEYKCKVSNKGLPSSIEKTISKAKGQPREPQVYTLPPSQEEMTKN |
| | QVSLTCLVKGFYPSDIAVEWESNGQPENNYKTTPPVLDSDGSFFLYSKLTVDKSRWQEGNVFSCSVMHEALHNHYTQKSL |
| | SLSLG |
| 110 | SRGIKGKRQRRISAEGSQACAKGCELCSEVNGCLKCSPKLFILLERNDIRQVGVCLPSCPPGYFDARNPDMNKCIKCKIEHCE |
| | ACFSHNFCTKCKEGLYLHKGRCYPACPEGSSAANGTMECSSPAQCEMSEWSPWGPCSKKQQLCGFRRGSEERTRRVLHA |
| | PVGDHAACSDTKETRRCTVRRVPCPEGQKRRKGGQGRRENANRNLARKESKEAGAGSRRRKGQQQQQQQGTVGPLTS |
| | AGPAGGGSGGGGSAESKYGPPCPPCPAPEAAGGPSVFLFPPKPKDTLMISRTPEVTCVVVDVSQEDPEVQFNWYVDGVE |
| | VHNAKTKPREEQFNSTYRVVSVLTVLHQDWLNGKEYKCKVSNKGLPSSIEKTISKAKGQPREPQVYTLPPSQEEMTKNQVS |
| | LTCLVKGFYPSDIAVEWESNGQPENNYKTTPPVLDSDGSFFLYSKLTVDKSRWQEGNVFSCSVMHEALHNHYTQKSLSLSL |
| | G |
| 111 | SRGIKGKRQRRISAEGSQACAKGCELCSEVNGCLKCSPKLFILLERNDIRQVGVCLPSCPPGYFDARNPDMNKCIKCKIEHCE |
| | ACFSHNFCTKCKEGLYLHKGRCYPACPEGSSAANGTMECSSPAQCEMSEWSPWGPCSKKQQLCGFRRGSEERTRRVLHA |
| | PVGDHAACSDTKETRRCTVRRVPCPEGQKRRKGGQGRRENANRNLARKESKEAGAGSRRRKGQQQQQQQGTVGPLTS |
| | AGPASGGGGSAESKYGPPCPPCPAPEAAGGPSVFLFPPKPKDTLMISRTPEVTCVVVDVSQEDPEVQFNWYVDGVEVHN |
| | AKTKPREEQFNSTYRVVSVLTVLHQDWLNGKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSKLTVDKSRWQEGNVFSCSVMHEALHNHYTQKSLSLSLG |
| 112 | MGFPAAALLCALCCGLLAPAARA |

TABLE 12
USEFUL NUCLEOTIDE SEQUENCES
FOR PRACTICING THE INVENTION

| SEQ ID | Brief description |
|---|---|
| 30 | Full length Rspo1 (with SP) |
| 31 | Full length Rspo2 (with SP) |
| 32 | Full length Rspo4 (with SP) |
| 33 | Full Length Variant #005 w/o SP (Rspo1 withN137D) |
| 34 | Full Length Variant #008 w/o SP (Rspo1 with R66A) |
| 35 | Full length Variant #009 w/o SP (del aa 21-31 N-term Rspo1 |
| 36 | Full length Variant #034 w/o SP (Rspo2 FU1 23-94 and Rspo1 |
| | otherwise) |
| 37 | Full length Variant #035 w/o SP (Rspo2 FU1 and Rspo1 |
| | otherwise) |
| 38 | Full length Variant #047 (=#0034 but with aa substitution |
| | E49K V50D D68G D85G) |
| 39 | Full length Variant #051 (=#0034 but with 9 aa substitutions) |
| 40 | Full lenght Variant #014 w/o SP (=#014) |

TABLE 13
USEFUL NUCLEOTIDE SEQUENCES FOR PRACTICING THE INVENTION

| | |
|---|---|
| 30 | ATGAGACTGGGCCTGTGTGTGGTGGCTCTGGTGCTGTCCTGGACCCACCTGACCATCTCCTCCCGGG |
| | GAATCAAGGGAAAGCGGCAGCGGCGGATCTCTGCTGAGGGATCTCAGGCTTGCGCTAAGGGCTGC |
| | GAGCTGTGCTCCGAGGTGAACGGCTGCCTGAAGTGCTCCCCAAAGCTGTTCATCCTCCTCGAGCGG |
| | AACGATATCCGGCAAGTGGGCGTGTGCCTCCCTTCTTGCCCTCCTGGCTACTTCGACGCCCGGAACC |
| | CCGACATGAACAAGTGCATCAAGTGCAAGATCGAGCACTGCGAGGCCTGCTTCTCCCACAACTTCTG |
| | CACCAAGTGTAAGGAGGGCCTCTACCTCCACAAGGGCAGATGCTACCCTGCTTGCCCTGAGGGCTCT |
| | TCTGCCGCCAACGGCACCATGGAGTGCTCTTCCCCAGCCCAGTGCGAGATGTCTGAGTGGTCTCCCT |
| | GGGGACCCTGCTCTAAGAAGCAGCAGCTCTGCGGGTTCAGAAGAGGATCTGAAGAACGGACACGG |
| | CGCGTGCTCCATGCTCCTGTCGGAGATCACGCTGCTTGCTCCGATACAAAGGAGACACGGCGGTGC |
| | ACCGTGAGAAGAGTGCCTTGCCCCGAGGGCCAGAAGAGAAGAAAGGGAGGACAAGGAAGGCGCG |
| | AGAACGCTAACCGCAACCTCGCCAGGAAGGAGTCCAAGGAGGCTGGAGCCGGGTCACGAAGGAGG |
| | AAGGGACAGCAACAGCAACAGCAGCAGGGAACAGTGGGGCCTCTCACCTCTGCTGGGCCTGCTTGA |
| 31 | ATGCAGTTCCGGCTGTTCTCCTTCGCCCTGATCATCCTCAACTGCATGGACTACTCTCACTGTCAGGGA |
| | AACCGGTGGCGGCGGTCTAAGCGGGCTTCCTACGTGTCCAACCCCATCTGCAAGGGCTGCCTGTCCT |
| | GCTCCAAGGACAACGGCTGCTCCCGGTGCCAGCAGAAGCTGTTCTTCTTCCTGCGGCGGGAGGGAA |
| | TGCGGCAGTACGGAGAGTGCCTCCACTCCTGCCCTTCTGGCTACTACGGCCACAGAGCCCCTGACAT |
| | GAACAGATGCGCCCGCTGCCGCATCGAGAACTGCGACTCTTGCTTCTCCAAGGACTTCTGCACCAAG |
| | TGCAAGGTGGGCTTCTACCTCCACCGAGGACGGTGTTTTGACGAGTGCCCTGACGGCTTCGCTCCTC |
| | TCGAGGAGACCATGGAGTGTGTGGAGGGGTGTGAGGTGGGCCACTGGTCTGAGTGGGGAACATG |
| | CAGCCGCAACAACCGCACCTGTGGGTTCAAGTGGGGACTCGAGACACGCACACGCCAGATCGTGA |
| | AGAAGCCCGTGAAGGACACCATCCTGTGCCCTACCATCGCCGAGTCTCGCCGCTGCAAGATGACCA |
| | TGAGACACTGCCCTGGAGGGAAGAGAACCCCAAAGGCCAAGGAGAAGCGCAACAAGAAGAAGAA |
| | GCGCAAGCTCATCGAGCGCGCTCAGGAGCAGCACAGCGTGTTCCTCGCTACCGACAGAGCTAACCA |
| | GGGTGGCGGTGGCTCGGAGCCTGAGGCTCACCATCACCATCACCACTAG |
| 32 | ATGAAGTGGGTCACCTTCATCTCCCTGCTCTTCCTCTTCTCCAGCGCTTACAGCCTGAACCGGCGGAA |
| | GAAGCAAGTGGGAACAGGCCTGGGCGGCAACTGTACAGGCTGCATCATCTGCTCCGAGGAGAACG |
| | GCTGCTCCACCTGCCAGCAGCGGCTGTTCCTGTTCATTCGGCGGGAAGGAATCCGGCAGTACGGCA |
| | AGTGCCTCCACGACTGTCCTCCTGGGTACTTTGGCATCCGGGGCCAGGAGGTGAACCGGTGTAAGA |
| | AGTGCGGCGCCACCTGCGAGTCCTGCTTCTCCCAGGACTTCTGCATCCGGTGCAAGCGCCAGTTCTA |
| | CCTCTACAAGGGGAAGTGCCTCCCTACCTGTCCTCCTGGGACCCTCGCTCACCAGAACACCCGCGAG |
| | TGCCAGGGAGAGTGTGAGCTCGGACCTTGGGGAGGCTGGTCTCCTTGTACCCACAACGGGAAGAC |
| | CTGTGGGTCTGCTTGGGGACTCGAGTCTAGAGTCCGCGAGGCTGGAAGAGCTGGACACGAGGAGG |
| | CTGCTACCTGTCAGGTGCTCTCCGAGAGCCGCAAGTGCCCCATCCAGAGACCTTGCCCTGGGGAGA |
| | GAAGTCCCGGGCAGAAGAAGGGACGCAAGGATCGGCGCCCTCGCAAGGATCGCAAGCTCGACAG |
| | AAGACTCGACGTGAGACCCAGACAGCCTGGACTCCAGCCTGGTGGCGGTGGCTCGGAGCCTGAGG |
| | CTCACCATCACCATCACCACTAG |
| 33 | ATGGTGTCCCAGGCTCTGAGACTGCTGTGTCTGCTGCTGGGACTGCAGGGCTGTCTGGCTTCTAGAG |
| | GCATCAAGGGAAAGCGGCAGCGGCGGATCTCTGCTGAGGGCTCTCAGGCTTGCGCTAAGGGCTGCG |
| | AGCTGTGCTCCGAGGTGAACGGCTGCCTGAAGTGCTCCCCAAAGCTCTTCATCCTCCTCGAGCGGAA |
| | CGATATCCGGCAAGTGGGCGTGTGCCTCCCTTCTTGCCCTCCTGGCTACTTCGACGCCCGGAACCCCG |
| | ACATGAACAAGTGCATCAAGTGCAAGATCGAGCACTGCGAGGCCTGCTTCTCCCACAACTTCTGCAC |
| | CAAGTGTAAGGAGGGCCTCTACCTCCACAAGGGCAGATGCTACCCTGCTTGCCCTGAGGGCTCTTCT |
| | GCTGCTCAGGGAACCATGGAGTGCTCTTCCCCAGCCCAGTGCGAGATGTCTGAGTGGTCTCCCTGGG |
| | GACCCTGCTCTAAGAAGCAGCAGCTCTGCGGGTTCAGAAGAGGATCTGAAGAACGGACACGGCGG |
| | GTGCTCCATGCTCCTGTCGGAGATCACGCTGCTTGCTCCGATACAAAGGAGACACGGCGGTGCACCG |
| | TGAGAAGAGTGCCTTGCCCCGAGGGGCAGAAGAGAAGAAAGGGAGGACAAGGAAGACGCGAGAA |
| | CGCTAACCGCAACCTCGCCAGGAAGGAGTCCAAGGAGGCTGGAGCCGGGTCACGAAGGAGGAAGG |
| | GGCAGCAACAGCAACAGCAGCAGGGAACAGTGGGGCCTCTCACATCTGCTGGACCAGCTGGTGGCG |
| | GTGGCTCGGAGCCTGAGGCTCACCATCACCATCACCACTGA |
| 34 | ATGGTGTCCCAGGCTCTGAGACTGCTGTGTCTGCTGCTGGGACTGCAGGGCTGTCTGGCTTCTAGAG |
| | GCATCAAGGGAAAGCGGCAGCGGCGGATCTCTGCTGAGGGCTCTCAGGCTTGCGCTAAGGGCTGC |
| | GAGCTGTGCTCCGAGGTGAACGGCTGCCTGAAGTGCTCCCCAAAGCTCTTCATCCTCCTCGAGGCTA |
| | ACGATATCCGGCAAGTGGGCGTGTGCCTCCCTTCTTGCCCTCCTGGCTACTTCGACGCCCGGAACCC |
| | CGACATGAACAAGTGCATCAAGTGCAAGATCGAGCACTGCGAGGCCTGCTTCTCCCACAACTTCTGC |
| | ACCAAGTGTAAGGAGGGCCTCTACCTCCACAAGGGCAGATGCTACCCTGCTTGCCCTGAGGGCTCTT |
| | CTGCCGCCAACGGAACCATGGAGTGCTCTTCCCCAGCCCAGTGCGAGATGTCTGAGTGGTCTCCCTG |
| | GGGACCCTGCTCTAAGAAGCAGCAGCTCTGCGGGTTCAGAAGAGGATCTGAAGAACGGACACGGC |
| | GGGTGCTCCATGCTCCTGTCGGAGATCACGCTGCTTGCTCCGATACAAAGGAGACACGGCGGTGCA |
| | CCGTGAGAAGAGTGCCTTGCCCCGAGGGCCAGAAGAGAAGAAAGGGAGGACAAGGAAGACGCGA |
| | GAACGCTAACCGCAACCTCGCCAGGAAGGAGTCCAAGGAGGCTGGAGCCGGGTCACGAAGGAGG |
| | AAGGGGCAGCAACAGCAACAGCAGCAGGGAACAGTGGGGCCTCTCACATCTGCTGGACCAGCTGGTGG |
| | CGGTGGCTCGGAGCCTGAGGCTCACCATCACCATCACCACTGA |
| 35 | ATGGTGTCTCAGGCCCTGAGACTGCTGTGCCTGCTGCTGGGACTGCAGGGCTGTCTGGCTATCTCCG |
| | CTGAGGGCTCTCAGGCTTGCGCTAAGGGCTGCGAGCTGTGCTCCGAGGTGAACGGCTGCCTGAAGT |
| | GCTCCCCAAAGCTCTTCATCCTCCTCGAGCGGAACGATATCCGGCAAGTGGGCGTGTGCCTCCCTTCT |
| | TGCCCTCCTGGCTACTTCGACGCCCGGAACCCCGACATGAACAAGTGCATCAAGTGCAAGATCGAGC |
| | ACTGCGAGGCCTGCTTCTCCCACAACTTCTGCACCAAGTGTAAGGAGGGCCTCTACCTCCACAAGGG |
| | CAGATGCTACCCTGCTTGCCCTGAGGGCTCTTCTGCCGCCAACGGCACCATGGAGTGCTCTTCCCCA |
| | GCCCAGTGCGAGATGTCTGAGTGGTCTCCCTGGGGACCCTGCTCTAAGAAGCAGCAGCTCTGCGGGT |
| | TCAGAAGAGGATCTGAAGAACGGACACGGCGGGTGCTCCATGCTCCTGTCGGAGATCACGCTGCTTG |
| | CTCCGATACAAAGGAGACCCGGCGGTGCACCGTGAGAAGAGTGCCTTGCCCCGAGGGCCAGAAGAG |
| | AAGAAAGGGAGGACAAGGAAGACGCGAGAACGCTAACCGCAACCTCGCCAGGAAGGAGTCCAAGG |
| | AGGCTGGAGCCGGCAGTCGAAGGAGGAAGGGGCAGCAACAGCAACAACAACAGGGAACAGTGGG |
| | GCCTCTCACATCTGCTGGACCTGCTGGTGGCGGTGGCTCGGAGCCTGAGGCTCACCATCACCATCAC |
| | CACTGA |
| 36 | ATGCAGTTCCGGCTGTTCTCCTTCGCCCTGATCATCCTCAACTGCATGGACTACTCCCACTGCGCCTC |
| | CTACGTGTCCAACCCCATCTGCAAGGGCTGCCTGTCCTGCTCCAAGGACAACGGCTGCTCCCGGTGC |
| | CAGCAGAAGCTGTTCTTCTTCCTCCGGCGGGAGGGAATGCGGCAGTACGGAGAGTGCCTGCACTCC |
| | TGCCCTTCTGGCTACTACGGCCACAGAGCCCCTGACATGAACCGGTGCGCCAAGTGCAAGATCGAG |
| | CACTGCGAGGCCTGCTTCTCCCACAACTTCTGCACCAAGTGCAAGGAGGGCCTGTACCTGCACAAG |
| | GGCAGATGCTATCCTGCTTGCCCTGAGGGCTCTTCTGCCGCCAACGGCACCATGGAGTGCTCTTCCC |
| | CAGCCCAGTGCGAGATGTCTGAGTGGTCTCCCTGGGGACCCTGCTCTAAGAAGCAGCAGCTGTGCG |
| | GATTCAGAAGAGGATCTGAAGAACGGACACGGCGGGTGCTGCATGCTCCTGTCGGAGATCACGCTG |
| | CTTGCTCCGATACAAAGGAGACCCGCCGCTGCACCGTGAGAAGAGTGCCTTGTCCCGAAGGCCAGA |
| | AGAGAAGAAAGGGTGGACAAGGAAGACGCGAGAACGCTAACCGCAACCTGGCCAGGAAGGAGAG |
| | CAAGGAGGCTGGGGCTGGGTCTAGGAGGAGGAAGGGGCAGCAACAGCAACAACAGCAGGGAAC |
| | AGTGGGGCCTCTGACATCTGCTGGACCAGCTGGTGGCGGTGGCAGCGAGCCTGAGGCTCACCATC |
| | ACCATCACCACTAG |
| 37 | ATGCAGTTCCGGCTGTTCTCCTTCGCCCTGATCATCCTCAACTGCATGGACTACTCCCACTGCGCCT |
| | CCTACGTGTCCAACCCCATCTGCAAGGGCTGCCTGTCCTGCTCCAAGGACAACGGCTGCTCCCGGT |
| | GCCAGCAGAAGCTGTTCTTCTTCCTCCGGCGGGAGGGAATGCGGCAGTACGGAGAGTGCCTGC |
| | ACTCCTGCCCTTCTGGCTACTACGGCCACAGAGCCCCAGACATGAACAAGTGCATCAAGTGCAAG |
| | ATCGAGCACTGCGAGGCCTGCTTCTCCCACAACTTCTGCACCAAGTGCAAGGAGGGCCTGTACCT |
| | GCACAAGGGCAGATGCTATCCTGCTTGCCCTGAGGGCTCTTCTGCCGCCAACGGCACCATGGAGT |
| | GCTCTTCCCCAGCCCAGTGCGAGATGTCTGAGTGGTCTCCCTGGGGACCCTGCTCTAAGAAGCAG |
| | CAGCTGTGCGGATTCAGAAGAGGATCTGAAGAACGGACACGGCGGGTGCTGCATGCTCCTGTCG |
| | GAGATCACGCTGCTTGCTCCGATACAAAGGAGACCCGCCGCTGCACCGTGAGAAGAGTGCCTTGT |
| | CCCGAAGGCCAGAAGAGAAGAAAGGGTGGACAAGGAAGGCGCGAGAACGCTAACCGCAACCTC |
| | GCCAGGAAGGAGAGCAAGGAGGCAGGCGCTGGGTCTAGGAGGAGGAAGGGGCAGCAACAGCA |
| | ACAACAGCAGGGAACAGTGGGGCCTCTGACATCTGCTGGACCAGCTGGTGGCGGTGGCAGCGAG |
| | CCTGAGGCTCACCATCACCATCACCACTAG |
| 38 | ATGAGACTGGGCCTGTGTGTGGTGGCTCTGGTGCTGTCCTGGACCCACCTGACCATCTCCATCTCC |
| | GCTGAGGGCTCTCAGGCTTGCGCTAAGGGCTGCGAGCTGTGCTCCAAGGACAACGGCTGCCTGA |
| | AGTGCTCCCCAAAGCTGTTCATCCTCCTCGAGCGGAACGGCATCAGACAGGTGGGCGTGTGCCTC |
| | CCTTCTTGCCCTCCTGGCTACTTCGGAGCTCGGAACCCTGACATGAACAAGTGCATCAAGTGCAAG |
| | ATCGAGCACTGCGAGGCCTGCTTCTCCCACAACTTCTGCACCAAGTGTAAGGAGGGCCTCTACCTC |
| | CACAAGGGCAGATGCTACCCTGCTTGCCCTGAGGGATCTTCTGCCGCCAACGGAACCATGGAGTG |
| | CTCTTCCCCAGCCCAGTGCGAGATGTCTGAGTGGTCTCCCTGGGGACCCTGCTCTAAGAAGCAGCA |
| | GCTCTGCGGGTTCAGAAGAGGATCTGAAGAACGGACACGGCGGGTGCTCCATGCTCCTGTCGGA |
| | GATCACGCTGCTTGCTCCGATACAAAGGAGACCCGGCGGTGCACCGTGAGAAGAGTGCCTTGCCC |
| | CGAGGGGCAGAAGAGACGAAAGGGAGGACAAGGAAGACGGGAGAACGCTAACCGCAACCTCG |
| | CCAGGAAGGAGTCCAAGGAGGCTGGAGCCGGCAGTCGAAGGAGGAAGGGGCAGCAACAGCAA |
| | CAGCAGCAGGGAACAGTGGGGCCTCTCACATCTGCTGGACCAGCTGGTGGCGGTGGCTCGGAGC |
| | CTGAGGCTCACCATCACCATCACCACTAG |
| 39 | ATGAGACTGGGCCTGTGTGTGGTGGCTCTGGTGCTGTCCTGGACCCACCTGACCATCTCCATCTCC |
| | GCTGAGGGCTCTCAGGCTTGCGCTAAGGGCTGCCTGCTGTGCTCCAAGGACAACGGCTGCCTGCG |
| | GTGCTCCCCAAAGCTGTTCATCCTCCTCGAGCGGAACGGAATCCGGCAAGTGGGAGTGTGTCTCC |
| | CTTCTTGCCCTCCTGGCTACTTCGGGGCCAGAGCCCCTGACATGAACAAGTGCATCAAGTGCAAGA |
| | TCGAGCACTGCGAGGCCTGCTTCTCCAAGGACTTCTGCACCAAGTGTAAGGAGGGCCTCTACCTCC |
| | ACAAGGGCAGATGCTACCCTGCTTGCCCTGAGGGCTCTTCTGCCGCCAACGGCACCATGGAGTGCT |
| | CTTCCCCAGCCCAGTGCGAGATGTCTGAGTGGTCTCCCTGGGGACCCTGCTCTAAGAAGCAGCAGC |
| | TCTGCGGGTTCAGAAGAGGATCTGAAGAACGGACACGGCGGGTGCTCCATGCTCCTGTCGGAGAT |
| | CACGCTGCTTGCTCCGATACAAAGGAGACCCGGCGGTGCACCGTGAGAAGAGTGCCTTGCCCCGA |
| | GGGCCAGAAGAGAAGGAAGGGAGGACAAGGAAGACGCGAGAACGCTAACCGCAACCTCGCCAG |
| | GAAGGAGTCCAAGGAGGCTGGAGCCGGCAGTCGAAGGAGGAAGGGGCAGCAACAGCAACAGCA |
| | GCAGGGAACAGTGGGGCCTCTCACATCTGCTGGACCAGCTGGTGGCGGTGGCTCGGAGCCTGAG |
| | GCTCACCATCACCATCACCACTAG |
| 40 | ATGAGACTGGGCCTGTGTGTGGTGGCTCTGGTGCTGTCCTGGACCCACCTGACCATCTCCTCCCGG |
| | GGAATCAAGGGAAAGCGGCAGCGGCGGATCTCTGCTGAGGGATCTCAGGCTTGCGCTAAGGGCT |
| | GCGAGCTGTGCTCCGAGGTGAACGGCTGCCTGAAGTGCTCCCCAAAGCTGTTCATCCTCCTCGAGC |
| | GGAACGATATCCGGCAAGTGGGCGTGTGCCTCCCTTCTTGCCCTCCTGGCTACTTCGACGCCCGGA |
| | ACCCCGACATGAACAAGTGCATCAAGTGCAAGATCGAGCACTGCGAGGCCTGCTTCTCCCACAACT |
| | TCTGCACCAAGTGTAAGGAGGGCCTCTACCTCCACAAGGGCAGATGCTACCCTGCTTGCCCTGAGG |
| | GCTCTTCTGCCGCCAACGGCACCATGGAGTGCTCTTCCCCAGCCCAGTGCGAGATGTCTGAGTGGT |
| | CTCCCTGGGGACCCTGCTCTAAGAAGCAGCAGCTCTGCGGGTTCAGAAGAGGATCTGAAGAACGG |
| | ACACGGCGCGTGCTCCATGCTCCTGTCGGAGATCACGCTGCTTGCTCCGATACAAAGGAGACACGG |
| | CGGTGCACCGTGAGAAGAGTGCCTTGCCCCGAGGGCCAGAAGAGAAGAAAGGGAGGACAAGGAA |
| | GGCGCGAGAACGCTAACCGCAACCTCGCCAGGAAGGAGTCCAAGGAGGCTGGAGCCGGGTCACG |
| | AAGGAGGAAGGGACAGCAACAGCAACAGCAGCAGGGAACAGTGGGGCCTCTCACCTCTGCTGGGC |
| | CTGCTTGA |

1. A recombinant variant of R-spondin protein comprising the following FU1, FU2, TSP and BR domains, wherein
a. FU1 is a domain having at least 80% identity to any of the FU1 domains of human Rspo1, Rspo2, Rspo3 or Rspo4 as shown in SEQ ID NO: 5, 9, 13 and 17, respectively,
b. FU2 is a domain having at least 80% identity to any of the FU2 domains of human Rspo1, Rspo2, Rspo3 or Rspo4 as shown in SEQ ID NO: 6, 10, 14, and 18, respectively,
c. TSP is a domain having at least 80% identity to any of the TSP domains of human Rspo1, Rspo2, Rspo3 or Rspo4 as shown in SEQ ID NO: 7, 11, 15, and 19, respectively, and,
d. BR is a domain having at least 80% identify to any of BR domains of human Rspo1, Rspo2, Rspo3, or Rspo4 as shown in SEQ ID NO: 8, 12, 16, and 20, respectively.

2. The recombinant variant of R-spondin protein of claim 1 comprising the Rspo1 FU1 and Rspo1 FU2 domains with at least H108K and N109D amino acid substitutions, preferably said variant comprises or essentially consists of SEQ ID NO:66.

3. The recombinant variant of claim 1, which has no more than 1, 2, 3, 4, 5, 6, 7, 8, 9, or 10 amino acid substitutions in each of the FU1 or FU2 domain when aligned with corresponding human Rspo1 FU1 domain of SEQ ID NO:5 and Rspo1 FU2 domain of SEQ ID NO:6 respectively.

4. The recombinant variant of claim 1, which comprises a deletion of the first 10-14 N-terminal amino acids within the region 21-33 of Rspo1, or within an equivalent region in Rspo2, Rspo3, or Rspo4, typically a deletion of residues 21-31 of Rspo1, preferably comprising or consisting of a protein of SEQ ID NO:24.

5. The recombinant variant of claim 1, which comprises at least an amino acid substitution of R66 in human Rspo1 FU1 domain of SEQ ID NO:5, or of the equivalent arginine residue in human Rspo2, Rspo3, or Rspo4 FU1 domain sequence, said amino acid substitution decreasing or abolishing ZNRF3 binding.

6. The recombinant variant of claim 1, wherein said variant does not comprise an N-glycosylation site between the FU2 and TSP domain.

7. The recombinant variant of claim 1, wherein said FU1 domain is 100% identical to Rspo2 FU1 domain of SEQ ID NO:9 and said FU2 is selected among FU2 domains of Rspo1, Rspo3, Rspo4, or their functional variants with amino acid substitutions maintaining at least the same binding affinity to LGR4.

8. The recombinant variant of claim 1, which exhibit one or more of the following properties to a level at least similar to Rspo1 protein of SEQ ID NO: 41:
(i) it binds to LGR4 receptor with at least the same affinity as reference human Rspo1 of SEQ ID NO:41, as measured in Rspo1/LGR4 binding affinity in vitro assay;
(ii) it induces the proliferation of functional beta-cells to at least a similar level as reference human Rspo1 of SEQ ID NO:41; and/or,
(iii) it induces the proliferation of functional beta-cells to at least a similar level as reference human Rspo1 of SEQ ID NO:41.

9. An Fc fusion protein, wherein the Fc fusion protein comprises a recombinant variant of R-spondin protein comprising the following FU1, FU2, TSP and BR domains, wherein
a. FU1 is a domain having at least 80% identity to any of the FU1 domains of human Rspo1, Rspo2, Rspo3 or Rspo4 as shown in SEQ ID NO: 5, 9, 13 and 17, respectively,
b. FU2 is a domain having at least 80% identity to any of the FU2 domains of human Rspo1, Rspo2, Rspo3 or Rspo4 as shown in SEQ ID NO: 6, 10, 14, and 18, respectively,
c. TSP is a domain having at least 80% identity to any of the TSP domains of human Rspo1, Rspo2, Rspo3 or Rspo4 as shown in SEQ ID NO: 7, 11, 15, and 19, respectively, and,
d. BR is a domain having at least 80% identify to any of BR domains of human Rspo1, Rspo2, Rspo3, or Rspo4 as shown in SEQ ID NO: 8, 12, 16, and 20, respectively,
and an Fc fragment fused directly, or indirectly via a peptide linker, to said recombinant variant, preferably said Fc fusion protein comprises or essentially consists of SEQ ID NO:42, 43, 92 or 93.

10. The Fc fusion protein of claim 9, wherein an Fc fragment of SEQ ID NO: 29 is fused via a peptidic linker at the N-terminal end of a variant of SEQ ID NO: 24 comprising or consisting of an amino acid sequence of SEQ ID NO: 96 or 97.

11. The Fc fusion protein of claim 9, wherein an Fc fragment of SEQ ID NO: 29 is fused via a peptidic linker at the N-terminal end of a variant of SEQ ID NO: 66 comprising or consisting of an amino acid sequence of SEQ ID NO: 98.

12-15. (canceled)

16. A method for treating diabetes, preferably diabetes type I or II, in a subject in need thereof, wherein the method comprises administering to said subject a therapeutically efficient amount of a recombinant variant of claim 1.

17. A nucleic acid, wherein the nucleic acid encodes a recombinant variant of R-spondin protein comprising the following FU1, FU2, TSP and BR domains, wherein
a. FU1 is a domain having at least 80% identity to any of the FU1 domains of human Rspo1, Rspo2, Rspo3 or Rspo4 as shown in SEQ ID NO: 5, 9, 13 and 17, respectively,
b. FU2 is a domain having at least 80% identity to any of the FU2 domains of human Rspo1, Rspo2, Rspo3 or Rspo4 as shown in SEQ ID NO: 6, 10, 14, and 18, respectively,
c. TSP is a domain having at least 80% identity to any of the TSP domains of human Rspo1, Rspo2, Rspo3 or Rspo4 as shown in SEQ ID NO: 7, 11, 15, and 19, respectively, and,
d. BR is a domain having at least 80% identify to any of BR domains of human Rspo1, Rspo2, Rspo3, or Rspo4 as shown in SEQ ID NO: 8, 12, 16, and 20, respectively.

18. A vector comprising a nucleic acid of claim 17.

19. A host cell, comprising a nucleic acid of claim 17.

20. A method for producing a recombinant variant of claim 1, comprising (i) culturing a host cell under conditions for expression of said recombinant variant, (ii) recovering said recombinant variant, and (iii) optionally purifying said recombinant variant.

21. A method for treating diabetes, preferably diabetes type I or II, in a subject in need thereof, wherein the method comprises administering to said subject a therapeutically efficient amount of an Fc fusion protein of claim 9.

22. A nucleic acid, wherein the nucleic acid encodes an Fc fusion protein, wherein the Fc fusion protein comprises a recombinant variant of R-spondin protein comprising the following FU1, FU2, TSP and BR domains, wherein
a. FU1 is a domain having at least 80% identity to any of the FU1 domains of human Rspo1, Rspo2, Rspo3 or Rspo4 as shown in SEQ ID NO: 5, 9, 13 and 17, respectively,
b. FU2 is a domain having at least 80% identity to any of the FU2 domains of human Rspo1, Rspo2, Rspo3 or Rspo4 as shown in SEQ ID NO: 6, 10, 14, and 18, respectively,
c. TSP is a domain having at least 80% identity to any of the TSP domains of human Rspo1, Rspo2, Rspo3 or Rspo4 as shown in SEQ ID NO: 7, 11, 15, and 19, respectively, and,
d. BR is a domain having at least 80% identify to any of BR domains of human Rspo1, Rspo2, Rspo3, or Rspo4 as shown in SEQ ID NO: 8, 12, 16, and 20, respectively,
and an Fc fragment fused directly, or indirectly via a peptide linker, to said recombinant variant, preferably said Fc fusion protein comprises or essentially consists of SEQ ID NO:42, 43, 92 or 93.

23. A vector comprising a nucleic acid of claim 22.

24. A host cell, comprising a nucleic acid of claim 22.

25. A method for producing an Fc fusion protein of claim 9, comprising (i) culturing a host cell under conditions for expression of said Fc fusion protein, (ii) recovering said Fc fusion protein, and (iii) optionally purifying said Fc fusion protein.


SEQUENCE LISTING (112 sequences)

SEQ ID NO: 1 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 2 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 3 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 4 - protein, 233 aa, Homo sapiens
MKWVTFISLLFLFSSAYSLNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 5 - protein, 52 aa, Homo sapiens
AEGSQACAKGCELCSEVNGCLKCSPKLFILLERNDIRQVGVCLPSCPPGYFD

SEQ ID NO: 6 - protein, 45 aa, Homo sapiens
MNKCIKCKIEHCEACFSHNFCTKCKEGLYLHKGRCYPACPEGSSA

SEQ ID NO: 7 - protein, 65 aa, Homo sapiens
SSPAQCEMSEWSPWGPCSKKQQLCGFRRGSEERTRRVLHAPVGDHAACSDTKETRRCTVRRVPCP

SEQ ID NO: 8 - protein, 56 aa, Homo sapiens
EGQKRRKGGQGRRENANRNLARKESKEAGAGSRRRKGQQQQQQQGTVGPLTSAGPA

SEQ ID NO: 9 - protein, 52 aa, Homo sapiens
YVSNPICKGCLSCSKDNGCSRCQQKLFFFLRREGMRQYGECLHSCPSGYYGH

SEQ ID NO: 10 - protein, 45 aa, Homo sapiens
NRCARCRIENCDSCFSKDFCTKCKVGFYLHRGRCFDECPDGFAPL

SEQ ID NO: 11 - protein, 60 aa, Homo sapiens
GCEVGHWSEWGTCSRNNRTCGFKWGLETRTRQIVKKPVKDTILCPTIAESRRCKMTMRHC

SEQ ID NO: 12 - protein, 40 aa, Homo sapiens
PGGKRTPKAKEKRNKKKKRKLIERAQEQHSVFLATDRANQ

SEQ ID NO: 13 - protein, 52 aa, Homo sapiens
HPNVSQGCQGGCATCSDYNGCLSCKPRLFFALERIGMKQIGVCLSSCPSGYY

SEQ ID NO: 14 - protein, 45 aa, Homo sapiens
DINKCTKCKADCDTCFNKNFCTKCKSGFYLHLGKCLDNCPEGLEA

SEQ ID NO: 15 - protein, 61 aa, Homo sapiens
HCEVSEWNPWSPCTKKGKTCGFKRGTETRVREIIQHPSAKGNLCPPTNETRKCTVQRKKCQ

SEQ ID NO: 16 - protein, 65 aa, Homo sapiens
KGERGKKGRERKRKKPNKGESKEAIPDSKSLESSKEIPEQRENKQQQKKRKVQDKQKSVSVSTVH

SEQ ID NO: 17 - protein, 52 aa, Homo sapiens
NCTGCIICSEENGCSTCQQRLFLFIRREGIRQYGKCLHDCPPGYFGIRGQEV

SEQ ID NO: 18 - protein, 45 aa, Homo sapiens
CGATCESCFSQDFCIRCKRQFYLYKGKCLPTCPPGTLAHQNTREC

SEQ ID NO: 19 - protein, 59 aa, Homo sapiens
ECELGPWGGWSPCTHNGKTCGSAWGLESRVREAGRAGHEEAATCQVLSESRKCPIQRPC

SEQ ID NO: 20 - protein, 38 aa, Homo sapiens
PGERSPGQKKGRKDRRPRKDRKLDRRLDVRPRQPGLQP

SEQ ID NO: 21 - protein, 52 aa, Homo sapiens
AEGSQACAKGCELCSEVNGCLKCSPKLFILLEANDIRQVGVCLPSCPPGYFD

SEQ ID NO: 22 - protein, 243 aa, synthetic construct
  REGION 1..243: note = Variant mutant Rspo1
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAAQGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 23 - protein, 243 aa, synthetic construct
  REGION 1..243: note = Variant Mutant Rspo1
SRGIKGKRQRRISAEGSQACAKGCELCSEVNGCLKCSPKLFILLEANDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 24 - protein, 232 aa, synthetic construct
  REGION 1..232: note = Variant mutant Rspo1
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 25 - protein, 231 aa, synthetic construct
  REGION 1..231: note = Variant mutant Rspo1
ASYVSNPICKGCLSCSKDNGCSRCQQKLFFFLRREGMRQYGECLHSCPSGYYGHRAPDMNRCAKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 26 - protein, 231 aa, synthetic construct
  REGION 1..231: note = Variant mutant Rspo1
ASYVSNPICKGCLSCSKDNGCSRCQQKLFFFLRREGMRQYGECLHSCPSGYYGHRAPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 27 - protein, 232 aa, synthetic construct
  REGION 1..232: note = Variant mutant Rspo1
ISAEGSQACAKGCELCSKDNGCLKCSPKLFILLERNGIRQVGVCLPSCPPGYFGARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 28 - protein, 232 aa, synthetic construct
  REGION 1..232: note = Variant mutant Rspo1
ISAEGSQACAKGCLLCSKDNGCLRCSPKLFILLERNGIRQVGVCLPSCPPGYFGARAPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 29 - protein, 228 aa, Homo sapiens
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 30 - DNA, 792 bp, Homo sapiens
  source 1..792: mol_type = other DNA
atgagactgggcctgtgtgtggtggctctggtgctgtcctggacccacctgaccatctcctcccggggaatcaagggaaagcggcagcggcggatctctgctgagggatctcaggcttgcgctaagggctgcgagctgtgctccgaggtgaacggctgcctgaagtgctccccaaagctgttcatcctcctcgagcggaacgatatccggcaagtgggcgtgtgcctcccttcttgccctcctggctacttcgacgcccggaaccccgacatgaacaagtgcatcaagtgcaagatcgagcactgcgaggcctgcttctcccacaacttctgcaccaagtgtaaggagggcctctacctccacaagggcagatgctaccctgcttgccctgagggctcttctgccgccaacggcaccatggagtgctcttccccagcccagtgcgagatgtctgagtggtctccctggggaccctgctctaagaagcagcagctctgcgggttcagaagaggatctgaagaacggacacggcgcgtgctccatgctcctgtcggagatcacgctgcttgctccgatacaaaggagacacggcggtgcaccgtgagaagagtgccttgccccgagggccagaagagaagaaagggaggacaaggaaggcgcgagaacgctaaccgcaacctcgccaggaaggagtccaaggaggctggagccgggtcacgaaggaggaagggacagcaacagcaacagcagcagggaacagtggggcctctcacctctgctgggcctgcttga

SEQ ID NO: 31 - DNA, 777 bp, Homo sapiens
  source 1..777: mol_type = other DNA
atgcagttccggctgttctccttcgccctgatcatcctcaactgcatggactactctcactgtcagggaaaccggtggcggcggtctaagcgggcttcctacgtgtccaaccccatctgcaagggctgcctgtcctgctccaaggacaacggctgctcccggtgccagcagaagctgttcttcttcctgcggcgggagggaatgcggcagtacggagagtgcctccactcctgcccttctggctactacggccacagagcccctgacatgaacagatgcgcccgctgccgcatcgagaactgcgactcttgcttctccaaggacttctgcaccaagtgcaaggtgggcttctacctccaccgaggacggtgttttgacgagtgccctgacggcttcgctcctctcgaggagaccatggagtgtgtggaggggtgtgaggtgggccactggtctgagtggggaacatgcagccgcaacaaccgcacctgtgggttcaagtggggactcgagacacgcacacgccagatcgtgaagaagcccgtgaaggacaccatcctgtgccctaccatcgccgagtctcgccgctgcaagatgaccatgagacactgccctggagggaagagaaccccaaaggccaaggagaagcgcaacaagaagaagaagcgcaagctcatcgagcgcgctcaggagcagcacagcgtgttcctcgctaccgacagagctaaccagggtggcggtggctcggagcctgaggctcaccatcaccatcaccactag

SEQ ID NO: 32 - DNA, 747 bp, Homo sapiens
  source 1..747: mol_type = other DNA
atgaagtgggtcaccttcatctccctgctcttcctcttctccagcgcttacagcctgaaccggcggaagaagcaagtgggaacaggcctgggcggcaactgtacaggctgcatcatctgctccgaggagaacggctgctccacctgccagcagcggctgttcctgttcattcggcgggaaggaatccggcagtacggcaagtgcctccacgactgtcctcctgggtactttggcatccggggccaggaggtgaaccggtgtaagaagtgcggcgccacctgcgagtcctgcttctcccaggacttctgcatccggtgcaagcgccagttctacctctacaaggggaagtgcctccctacctgtcctcctgggaccctcgctcaccagaacacccgcgagtgccagggagagtgtgagctcggaccttggggaggctggtctccttgtacccacaacgggaagacctgtgggtctgcttggggactcgagtctagagtccgcgaggctggaagagctggacacgaggaggctgctacctgtcaggtgctctccgagagccgcaagtgccccatccagagaccttgccctggggagagaagtcccgggcagaagaagggacgcaaggatcggcgccctcgcaaggatcgcaagctcgacagaagactcgacgtgagacccagacagcctggactccagcctggtggcggtggctcggagcctgaggctcaccatcaccatcaccactag

SEQ ID NO: 33 - DNA, 837 bp, synthetic construct
  misc_feature 1..837: note = Rspo1 mutant
  source 1..837: mol_type = other DNA
atggtgtcccaggctctgagactgctgtgtctgctgctgggactgcagggctgtctggcttctagaggcatcaagggaaagcggcagcggcggatctctgctgagggctctcaggcttgcgctaagggctgcgagctgtgctccgaggtgaacggctgcctgaagtgctccccaaagctcttcatcctcctcgagcggaacgatatccggcaagtgggcgtgtgcctcccttcttgccctcctggctacttcgacgcccggaaccccgacatgaacaagtgcatcaagtgcaagatcgagcactgcgaggcctgcttctcccacaacttctgcaccaagtgtaaggagggcctctacctccacaagggcagatgctaccctgcttgccctgagggctcttctgctgctcagggaaccatggagtgctcttccccagcccagtgcgagatgtctgagtggtctccctggggaccctgctctaagaagcagcagctctgcgggttcagaagaggatctgaagaacggacacggcgggtgctccatgctcctgtcggagatcacgctgcttgctccgatacaaaggagacacggcggtgcaccgtgagaagagtgccttgccccgaggggcagaagagaagaaagggaggacaaggaagacgcgagaacgctaaccgcaacctcgccaggaaggagtccaaggaggctggagccgggtcacgaaggaggaaggggcagcaacagcaacagcagcagggaacagtggggcctctcacatctgctggaccagctggtggcggtggctcggagcctgaggctcaccatcaccatcaccactga

SEQ ID NO: 34 - DNA, 837 bp, synthetic construct
  misc_feature 1..837: note = Mutant Rspo1
  source 1..837: mol_type = other DNA
atggtgtcccaggctctgagactgctgtgtctgctgctgggactgcagggctgtctggcttctagaggcatcaagggaaagcggcagcggcggatctctgctgagggctctcaggcttgcgctaagggctgcgagctgtgctccgaggtgaacggctgcctgaagtgctccccaaagctcttcatcctcctcgaggctaacgatatccggcaagtgggcgtgtgcctcccttcttgccctcctggctacttcgacgcccggaaccccgacatgaacaagtgcatcaagtgcaagatcgagcactgcgaggcctgcttctcccacaacttctgcaccaagtgtaaggagggcctctacctccacaagggcagatgctaccctgcttgccctgagggctcttctgccgccaacggaaccatggagtgctcttccccagcccagtgcgagatgtctgagtggtctccctggggaccctgctctaagaagcagcagctctgcgggttcagaagaggatctgaagaacggacacggcgggtgctccatgctcctgtcggagatcacgctgcttgctccgatacaaaggagacacggcggtgcaccgtgagaagagtgccttgccccgagggccagaagagaagaaagggaggacaaggaagacgcgagaacgctaaccgcaacctcgccaggaaggagtccaaggaggctggagccgggtcacgaaggaggaaggggcagcaacagcaacagcagcagggaacagtggggcctctcacatctgctggaccagctggtggcggtggctcggagcctgaggctcaccatcaccatcaccactga

SEQ ID NO: 35 - DNA, 804 bp, synthetic construct
  misc_feature 1..804: note = Mutant Rspo1
  source 1..804: mol_type = other DNA
atggtgtctcaggccctgagactgctgtgcctgctgctgggactgcagggctgtctggctatctccgctgagggctctcaggcttgcgctaagggctgcgagctgtgctccgaggtgaacggctgcctgaagtgctccccaaagctcttcatcctcctcgagcggaacgatatccggcaagtgggcgtgtgcctcccttcttgccctcctggctacttcgacgcccggaaccccgacatgaacaagtgcatcaagtgcaagatcgagcactgcgaggcctgcttctcccacaacttctgcaccaagtgtaaggagggcctctacctccacaagggcagatgctaccctgcttgccctgagggctcttctgccgccaacggcaccatggagtgctcttccccagcccagtgcgagatgtctgagtggtctccctggggaccctgctctaagaagcagcagctctgcgggttcagaagaggatctgaagaacggacacggcgggtgctccatgctcctgtcggagatcacgctgcttgctccgatacaaaggagacccggcggtgcaccgtgagaagagtgccttgccccgagggccagaagagaagaaagggaggacaaggaagacgcgagaacgctaaccgcaacctcgccaggaaggagtccaaggaggctggagccggcagtcgaaggaggaaggggcagcaacagcaacaacaacagggaacagtggggcctctcacatctgctggacctgctggtggcggtggctcggagcctgaggctcaccatcaccatcaccactga

SEQ ID NO: 36 - DNA, 804 bp, synthetic construct
  misc_feature 1..804: note = Rspo1 mutant
  source 1..804: mol_type = other DNA
atgcagttccggctgttctccttcgccctgatcatcctcaactgcatggactactcccactgcgcctcctacgtgtccaaccccatctgcaagggctgcctgtcctgctccaaggacaacggctgctcccggtgccagcagaagctgttcttcttcctccggcgggagggaatgcggcagtacggagagtgcctgcactcctgcccttctggctactacggccacagagcccctgacatgaaccggtgcgccaagtgcaagatcgagcactgcgaggcctgcttctcccacaacttctgcaccaagtgcaaggagggcctgtacctgcacaagggcagatgctatcctgcttgccctgagggctcttctgccgccaacggcaccatggagtgctcttccccagcccagtgcgagatgtctgagtggtctccctggggaccctgctctaagaagcagcagctgtgcggattcagaagaggatctgaagaacggacacggcgggtgctgcatgctcctgtcggagatcacgctgcttgctccgatacaaaggagacccgccgctgcaccgtgagaagagtgccttgtcccgaaggccagaagagaagaaagggtggacaaggaagacgcgagaacgctaaccgcaacctggccaggaaggagagcaaggaggctggggctgggtctaggaggaggaaggggcagcaacagcaacaacagcagggaacagtggggcctctgacatctgctggaccagctggtggcggtggcagcgagcctgaggctcaccatcaccatcaccactag

SEQ ID NO: 37 - DNA, 804 bp, synthetic construct
  misc_feature 1..804: note = Rspo1 mutant
  source 1..804: mol_type = other DNA
atgcagttccggctgttctccttcgccctgatcatcctcaactgcatggactactcccactgcgcctcctacgtgtccaaccccatctgcaagggctgcctgtcctgctccaaggacaacggctgctcccggtgccagcagaagctgttcttcttcctccggcgggagggaatgcggcagtacggagagtgcctgcactcctgcccttctggctactacggccacagagccccagacatgaacaagtgcatcaagtgcaagatcgagcactgcgaggcctgcttctcccacaacttctgcaccaagtgcaaggagggcctgtacctgcacaagggcagatgctatcctgcttgccctgagggctcttctgccgccaacggcaccatggagtgctcttccccagcccagtgcgagatgtctgagtggtctccctggggaccctgctctaagaagcagcagctgtgcggattcagaagaggatctgaagaacggacacggcgggtgctgcatgctcctgtcggagatcacgctgcttgctccgatacaaaggagacccgccgctgcaccgtgagaagagtgccttgtcccgaaggccagaagagaagaaagggtggacaaggaaggcgcgagaacgctaaccgcaacctcgccaggaaggagagcaaggaggcaggcgctgggtctaggaggaggaaggggcagcaacagcaacaacagcagggaacagtggggcctctgacatctgctggaccagctggtggcggtggcagcgagcctgaggctcaccatcaccatcaccactag

SEQ ID NO: 38 - DNA, 804 bp, synthetic construct
  misc_feature 1..804: note = Rspo1 mutant
  source 1..804: mol_type = other DNA
atgagactgggcctgtgtgtggtggctctggtgctgtcctggacccacctgaccatctccatctccgctgagggctctcaggcttgcgctaagggctgcgagctgtgctccaaggacaacggctgcctgaagtgctccccaaagctgttcatcctcctcgagcggaacggcatcagacaggtgggcgtgtgcctcccttcttgccctcctggctacttcggagctcggaaccctgacatgaacaagtgcatcaagtgcaagatcgagcactgcgaggcctgcttctcccacaacttctgcaccaagtgtaaggagggcctctacctccacaagggcagatgctaccctgcttgccctgagggatcttctgccgccaacggaaccatggagtgctcttccccagcccagtgcgagatgtctgagtggtctccctggggaccctgctctaagaagcagcagctctgcgggttcagaagaggatctgaagaacggacacggcgggtgctccatgctcctgtcggagatcacgctgcttgctccgatacaaaggagacccggcggtgcaccgtgagaagagtgccttgccccgaggggcagaagagacgaaagggaggacaaggaagacgggagaacgctaaccgcaacctcgccaggaaggagtccaaggaggctggagccggcagtcgaaggaggaaggggcagcaacagcaacagcagcagggaacagtggggcctctcacatctgctggaccagctggtggcggtggctcggagcctgaggctcaccatcaccatcaccactag

SEQ ID NO: 39 - DNA, 804 bp, synthetic construct
  misc_feature 1..804: note = Rspo1 mutant
  source 1..804: mol_type = other DNA
atgagactgggcctgtgtgtggtggctctggtgctgtcctggacccacctgaccatctccatctccgctgagggctctcaggcttgcgctaagggctgcctgctgtgctccaaggacaacggctgcctgcggtgctccccaaagctgttcatcctcctcgagcggaacggaatccggcaagtgggagtgtgtctcccttcttgccctcctggctacttcggggccagagcccctgacatgaacaagtgcatcaagtgcaagatcgagcactgcgaggcctgcttctccaaggacttctgcaccaagtgtaaggagggcctctacctccacaagggcagatgctaccctgcttgccctgagggctcttctgccgccaacggcaccatggagtgctcttccccagcccagtgcgagatgtctgagtggtctccctggggaccctgctctaagaagcagcagctctgcgggttcagaagaggatctgaagaacggacacggcgggtgctccatgctcctgtcggagatcacgctgcttgctccgatacaaaggagacccggcggtgcaccgtgagaagagtgccttgccccgagggccagaagagaaggaagggaggacaaggaagacgcgagaacgctaaccgcaacctcgccaggaaggagtccaaggaggctggagccggcagtcgaaggaggaaggggcagcaacagcaacagcagcagggaacagtggggcctctcacatctgctggaccagctggtggcggtggctcggagcctgaggctcaccatcaccatcaccactag

SEQ ID NO: 40 - DNA, 792 bp, Homo sapiens; identical to SEQ ID NO: 30
  source 1..792: mol_type = other DNA

SEQ ID NO: 41 - protein, 243 aa, Homo sapiens
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 42 - protein, 489 aa, synthetic construct
  REGION 1..489: note = Rspo1 fusion protein
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 43 - protein, 487 aa, synthetic construct
  REGION 1..487: note = Rspo1 fusion protein
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGGGSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 44 - protein, 18 aa, synthetic construct
  REGION 1..18: note = Peptidic linker
GGGGGGSGGGGSGGGGSA

SEQ ID NO: 45 - protein, 16 aa, synthetic construct
  REGION 1..16: note = Peptidic linker
GGGGSGGGGSGGGGGG

SEQ ID NO: 46 - protein, 5 aa, synthetic construct
  REGION 1..5: note = Peptidic linker
GGGGS

SEQ ID NO: 47 - protein, 232 aa, synthetic construct
  REGION 1..232: note = Rspo1 mutant
ISAEGSQACAKGCELCSERNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 48 - protein, 232 aa, synthetic construct
  REGION 1..232: note = Rspo1 mutant
ISAEGSQACAKGCELCSEVWGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 49 - protein, 120 aa, Homo sapiens
SPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 50 - protein, 231 aa, synthetic construct
  REGION 1..231: note = Rspo1 mutant
ISAEGSQACAKGCELCSEVNGCLKCSPKLFILLERNDIRQVGVCLPSCPPGYFDARNPDMNKCIKCKADCDTCFNKNFCTKCKSGFYLHLGKCLDNCPEGLEANNHTMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 51 - protein, 231 aa, synthetic construct
  REGION 1..231: note = Rspo1 mutant
ISAEGSQACAKGCELCSEVNGCLKCSPKLFILLERNDIRQVGVCLPSCPPGYFDARNPDINKCTKCKADCDTCFNKNFCTKCKSGFYLHLGKCLDNCPEGLEANNHTMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 52 - protein, 232 aa, synthetic construct
  REGION 1..232: note = Rspo1 mutant
MHPNVSQGCQGGCATCSDYNGCLSCKPRLFFALERIGMKQIGVCLSSCPSGYYGTRYPDINKCTKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 53 - protein, 232 aa, synthetic construct
  REGION 1..232: note = Rspo1 mutant
MHPNVSQGCQGGCATCSDYNGCLSCKPRLFFALERIGMKQIGVCLSSCPSGYYGTRYPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 54 - protein, 232 aa, synthetic construct
  REGION 1..232: note = Rspo1 mutant
MHPNVSQGCQGGCATCSDYNGCLSCKPRLFFALERIGMKQIGVCLSSCPSGYYGTRYPDINKCTKCKIEHCEACFSHNFCTKCKEGLYLHKGRCYPACPEGSSAAQGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 55 - protein, 231 aa, synthetic construct
  REGION 1..231: note = Rspo1 mutant
ASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 56 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIRCRIENCDSCFSKDFCTKCKVGFYLHRGRCFDECPDGFAPLEETMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 57 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNRCARCRIENCDSCFSKDFCTKCKVGFYLHRGRCFDECPDGFAPLEETMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 58 - protein, 232 aa, synthetic construct
  REGION 1..232: note = Rspo1 mutant
LNVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 59 - protein, 231 aa, synthetic construct
  REGION 1..231: note = Rspo1 mutant
ISAEGSQACAKGCELCSEVNGCLKCSPKLFILLERNDIRQVGVCLPSCPPGYFDARNPDMNKCIKCGATCESCFSQDFCIRCKRQFYLYKGKCLPTCPPGTLAHQNTRECQSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 60 - protein, 231 aa, synthetic construct
  REGION 1..231: note = Rspo1 mutant
ISAEGSQACAKGCELCSEVNGCLKCSPKLFILLERNDIRQVGVCLPSCPPGYFDARNPDVNRCKKCGATCESCFSQDFCIRCKRQFYLYKGKCLPTCPPGTLAHQNTRECQSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 61 - protein, 233 aa, synthetic construct
  REGION 1..233: note = Rspo1 mutant
LNVGTGLGGNCTGCIICSEENGCSTCQQRLFLFIRREGIRQYGKCLHDCPPGYFGIRGQEVNRCKKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 62 - protein, 233 aa, synthetic construct
  REGION 1..233: note = Rspo1 mutant
LNVGTGLGGNCTGCIICSEENGCSTCQQRLFLFIRREGIRQYGKCLHDCPPGYFGIRGQEMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 63 - protein, 230 aa, synthetic construct
  REGION 1..230: note = Rspo1 mutant
ASYVSNPICKGCLSCSKDNGCSRCQQKLFFFLRREGMRQYGECLHDCPPGYFGIRGQEVNRCKKCGATCESCFSQDFCIRCKRQFYLYKGKCLPTCPPGTLAHQNTRECQSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 64 - protein, 268 aa, synthetic construct
  REGION 1..268: note = Rspo1 mutant
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGGSQRLMEDICLPRWGCLWEDDF

SEQ ID NO: 65 - protein, 232 aa, synthetic construct
  REGION 1..232: note = Rspo1 mutant
ISAEGSQACAKGCLLCSKDNGCLRCSPKLFILLERNGIRQVGVCLPSCPPGYFGARAPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 66 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 67 - protein, 232 aa, synthetic construct
  REGION 1..232: note = Rspo1 mutant
ISAEGSQACAKGCELCSKDNGCLKCSPKLFILLERNGIRQVGVCLPSCPPGYFGARNPDMNKCIKCKIEHCEACFSKNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 68 - protein, 232 aa, synthetic construct
  REGION 1..232: note = Rspo1 mutant
ISAEGSQACAKGCELCSKDNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 69 - protein, 232 aa, synthetic construct
  REGION 1..232: note = Rspo1 mutant
ISAEGSQACAKGCELCSEVNGCLKCSPKLFILLERNGIRQVGVCLPSCPPGYFGARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 70 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSKNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 71 - protein, 232 aa, synthetic construct
  REGION 1..232: note = Rspo1 mutant
ISAEGSQACAKGCESCSEVNGCLKCQPKLFFFLERNDIRQVGVCLHSCPPGYYDARNPDMNKCAKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 72 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKVGFYLHKGRCYDECPEGFSALNETMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 73 - protein, 232 aa, synthetic construct
  REGION 1..232: note = Rspo1 mutant
ISAEGSQACAKGCESCSEVNGCLKCQPKLFFFLERNDIRQVGVCLHSCPPGYYDARNPDMNKCAKCKIEHCEACFSHNFCTKCKVGFYLHKGRCYDECPEGFSALNETMECVSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 74 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSRNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 75 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 76 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNAARKESKEAGAGSRRRKGQQQQQQQGTVGPLTSAGPA

SEQ ID NO: 77 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGAVGPLTSAGPA

SEQ ID NO: 78 - protein, 247 aa, synthetic construct
  REGION 1..247: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGAVGPLAAAGPAGGGGAEPEAHHHHHH

SEQ ID NO: 79 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQTTVGPLTSAGPA

SEQ ID NO: 80 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPSTSAGPA

SEQ ID NO: 81 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSTGPA

SEQ ID NO: 82 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPT

SEQ ID NO: 83 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQTTVGPSTSTGPT

SEQ ID NO: 84 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGEVGPLTSAGPA

SEQ ID NO: 85 - protein, 247 aa, synthetic construct
  REGION 1..247: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGEVGPLEEAGPAGGGGEEPEAHHHHHH

SEQ ID NO: 86 - protein, 232 aa, synthetic construct
  REGION 1..232: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGNGTNLTSAGPA

SEQ ID NO: 87 - protein, 279 aa, synthetic construct
  REGION 1..279: note = Rspo1 mutant
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGGSGGGGSGGGGGGQRLMEDICLPRWGCLWEDDF

SEQ ID NO: 88 - protein, 278 aa, synthetic construct
  REGION 1..278: note = Rspo1 mutant
QRLMEDICLPRWGCLWEDDFGGGGSGGGGSGGGGGGSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGP

SEQ ID NO: 89 - protein, 232 aa, synthetic construct
  REGION 1..232: note = Rspo1 mutant
ISAEGSQACAKGCELCSEVNGCLKCSPKLFILLEANDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 90 - protein, 15 aa, synthetic construct
  REGION 1..15: note = Polyhistidine tag
GGGGSEPEAHHHHHH

SEQ ID NO: 91 - protein, 11 aa, synthetic construct
  REGION 1..11: note = Peptide binding to serum albumin
DICLPRWGCLW

SEQ ID NO: 92 - protein, 500 aa, synthetic construct
  REGION 1..500: note = Rspo1 mutant
MALWMRFLPLLALLVLWEPKPAQAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGGGISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 93 - protein, 478 aa, synthetic construct
  REGION 1..478: note = Rspo1 mutant
ISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 94 - protein, 213 aa, synthetic construct
  REGION 1..213: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKG

SEQ ID NO: 95 - protein, 459 aa, synthetic construct
  REGION 1..459: note = Rspo1 mutant
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 96 - protein, 476 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGGGISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 97 - protein, 500 aa, synthetic construct
MALWMRFLPLLALLVLWEPKPAQAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGGGISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 98 - protein, 476 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGGGGGSGGGGSGGGGGGISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 99 - protein, 24 aa, synthetic construct
MALWMRFLPLLALLVLWEPKPAQA

SEQ ID NO: 100 - protein, 462 aa, synthetic construct
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPASAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 101 - protein, 462 aa, synthetic construct
ISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPASAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 102 - protein, 13 aa, synthetic construct
GGGGGGSGGGGSA

SEQ ID NO: 103 - protein, 10 aa, synthetic construct
GGGSGGGGSA

SEQ ID NO: 104 - protein, 7 aa, synthetic construct
SGGGGSA

SEQ ID NO: 105 - protein, 473 aa, synthetic construct
ISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 106 - protein, 475 aa, synthetic construct
ISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPASAGGGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 107 - protein, 472 aa, synthetic construct
ISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPASAGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 108 - protein, 468 aa, synthetic construct
ISAEGSQACAKGCELCSEVNGCLKCSPKLFILLERNDIRQVGVCLPSCPPGYFDARNPDMNKCIKCKIEHCEACFSKDFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPASASGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 109 - protein, 484 aa, synthetic construct
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 110 - protein, 481 aa, synthetic construct
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPAGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 111 - protein, 478 aa, synthetic construct
SRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPASGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 112 - protein, 23 aa, synthetic construct
MGFPAAALLCALCCGLLAPAARA